SEQUENCE LISTING (131 sequences)

SEQ ID NO: 1 - protein, 8 aa, synthetic construct
SAGSGLYG

SEQ ID NO: 2 - protein, 7 aa, synthetic construct
GTNKRPS

SEQ ID NO: 3 - protein, 11 aa, synthetic construct
GSADSSTNAGI

SEQ ID NO: 4 - protein, 5 aa, synthetic construct
SYAMN

SEQ ID NO: 5 - protein, 17 aa, synthetic construct
GISSSGRYTGYADSVKG

SEQ ID NO: 6 - protein, 18 aa, synthetic construct
SVGSGVSWSGYVATSLDA

SEQ ID NO: 7 - protein, 5 aa, synthetic construct
RYTMH

SEQ ID NO: 8 - protein, 17 aa, synthetic construct
YINPSRGYTNYNQKFKD

SEQ ID NO: 9 - protein, 10 aa, synthetic construct
YYDDHYCLDY

SEQ ID NO: 10 - protein, 10 aa, synthetic construct
RASSSVSYMN

SEQ ID NO: 11 - protein, 7 aa, synthetic construct
DTSKVAS

SEQ ID NO: 12 - protein, 9 aa, synthetic construct
QQWSSNPLT

SEQ ID NO: 13 - protein, 8 aa, synthetic construct
SGGSGSYG

SEQ ID NO: 14 - protein, 7 aa, synthetic construct
GTYKRPS

SEQ ID NO: 15 - protein, 18 aa, synthetic construct
SVGSGVSWSGYVATSLDV

SEQ ID NO: 16 - protein, 5 aa, synthetic construct
GYSMG

SEQ ID NO: 17 - protein, 17 aa, synthetic construct
AIVWSGGNTYYEDSVKG

SEQ ID NO: 18 - protein, 15 aa, synthetic construct
KIRPYIFKIAGQYDY

SEQ ID NO: 19 - protein, 5 aa, synthetic construct
KYAMN

SEQ ID NO: 20 - protein, 19 aa, synthetic construct
RIRSKYNNYATYYADSVKD

SEQ ID NO: 21 - protein, 14 aa, synthetic construct
HGNFGNSYISYWAY

SEQ ID NO: 22 - protein, 14 aa, synthetic construct
GSSTGAVTSGNYPN

SEQ ID NO: 23 - protein, 7 aa, synthetic construct
GTKFLAP

SEQ ID NO: 24 - protein, 9 aa, synthetic construct
VLWYSNRWV

SEQ ID NO: 25 - protein, 5 aa, synthetic construct
TYAMN

SEQ ID NO: 26 - protein, 19 aa, synthetic construct
RIRSKYNNYATYYADSVKG

SEQ ID NO: 27 - protein, 14 aa, synthetic construct
HGNFGDSYVSWFAY

SEQ ID NO: 28 - protein, 14 aa, synthetic construct
GSSTGAVTTSNYAN

SEQ ID NO: 29 - protein, 7 aa, synthetic construct
GTNKRAP

SEQ ID NO: 30 - protein, 9 aa, synthetic construct
ALWYSNHWV

SEQ ID NO: 31 - protein, 19 aa, synthetic construct
RIRSKYNNYATYYADSVKS

SEQ ID NO: 32 - protein, 14 aa, synthetic construct
HGNFGNSYVSWFAY

SEQ ID NO: 33 - protein, 14 aa, synthetic construct
RSSTGAVTTSNYAN

SEQ ID NO: 34 - protein, 7 aa, synthetic construct
GANKRAP

SEQ ID NO: 35 - protein, 9 aa, synthetic construct
ALWYSNLWV

SEQ ID NO: 36 - protein, 17 aa, synthetic construct
YINPSRGYTNYNQKVKD

SEQ ID NO: 37 - protein, 10 aa, synthetic construct
SASSSVSYMN

SEQ ID NO: 38 - protein, 7 aa, synthetic construct
DTSKLAS

SEQ ID NO: 39 - protein, 9 aa, synthetic construct
QQWSSNPFT

SEQ ID NO: 40 - protein, 5 aa, synthetic construct
GYTMN

SEQ ID NO: 41 - protein, 17 aa, synthetic construct
LINPYKGVSTYNQKVKG

SEQ ID NO: 42 - protein, 13 aa, synthetic construct
SGYYGDSDWYFDV

SEQ ID NO: 43 - protein, 11 aa, synthetic construct
RASQDIRNYLN

SEQ ID NO: 44 - protein, 7 aa, synthetic construct
YTSRLHS

SEQ ID NO: 45 - protein, 9 aa, synthetic construct
QQGNTLPWT

SEQ ID NO: 46 - protein, 6 aa, synthetic construct
HHHHHH

SEQ ID NO: 47 - protein, 18 aa, synthetic construct
SMGSGVSWSGYVATSIDV

SEQ ID NO: 48 - protein, 13 aa, synthetic construct
RSSTGAVTTSNYA

SEQ ID NO: 49 - protein, 13 aa, synthetic construct
GSSTGAVTSGNYP

SEQ ID NO: 50 - protein, 15 aa, synthetic construct
GGGGSGGGGSGGGGS

SEQ ID NO: 51 - protein, 5 aa, synthetic construct
GGGGS

SEQ ID NO: 52 - protein, 16 aa, synthetic construct
GGSGGSGGSGGSGGVD

SEQ ID NO: 53 - protein, 20 aa, synthetic construct
GKPGSGKPGSGKPGSGKPGS

SEQ ID NO: 54 - protein, 5 aa, synthetic construct
  REPEAT 1..5: note = sequence is present 1, 2, 3, or 4 times
GGGGS

SEQ ID NO: 55 - protein, 8 aa, synthetic construct
GGGGGGGG

SEQ ID NO: 56 - protein, 6 aa, synthetic construct
GGGGGG

SEQ ID NO: 57 - protein, 5 aa, synthetic construct
  REPEAT 1..5: note = Sequence is present 1, 2, or 3 times
EAAAK

SEQ ID NO: 58 - protein, 7 aa, synthetic construct
  REPEAT 2..6: note = Sequence is repeated 2, 3, 4, or 5 times
AEAAAKA

SEQ ID NO: 59 - protein, 12 aa, synthetic construct
AEAAAKEAAAKA

SEQ ID NO: 60 - protein, 16 aa, synthetic construct
  REPEAT 11..15: note = Sequence is repeated 4 times
  REPEAT 2..6: note = Sequence is repeated 4 times
AEAAAKALEAEAAAKA

SEQ ID NO: 61 - protein, 5 aa, synthetic construct
PAPAP

SEQ ID NO: 62 - protein, 18 aa, synthetic construct
KESGSVSSEQLAQFRSLD

SEQ ID NO: 63 - protein, 14 aa, synthetic construct
EGKSSGSGSESKST

SEQ ID NO: 64 - protein, 12 aa, synthetic construct
GSAGSAAGSGEF

SEQ ID NO: 65 - protein, 211 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 66 - protein, 211 aa, synthetic construct
SYELTQPPSVSVSPGQTARITCSGGSGSYGWYQQKPGQAPVLVIYGTYKRPSGIPERFSGSSSGTTVTLTISGVQAEDEADYYCGSADSSTNAGIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 67 - protein, 211 aa, synthetic construct; identical to SEQ ID NO: 65

SEQ ID NO: 68 - protein, 105 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVL

SEQ ID NO: 69 - protein, 127 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSS

SEQ ID NO: 70 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 71 - protein, 109 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 72 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 73 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 233 aa, synthetic construct
ASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 75 - protein, 124 aa, synthetic construct
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSS

SEQ ID NO: 76 - protein, 457 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 211 aa, synthetic construct; identical to SEQ ID NO: 65

SEQ ID NO: 78 - protein, 357 aa, synthetic construct
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSSASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 79 - protein, 457 aa, synthetic construct; identical to SEQ ID NO: 76

SEQ ID NO: 80 - protein, 357 aa, synthetic construct; identical to SEQ ID NO: 78

SEQ ID NO: 81 - protein, 454 aa, synthetic construct
EVQLLESGGGLVQPGGSLRLSCAASGFTYRGYSMGWVRQAPGKGLEFVAAIVWSGGNTYYEDSVKGRFTISRDNSKNTLYLQMNSLRAEDTAVYYCAAKIRPYIFKIAGQYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 82 - protein, 495 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 83 - protein, 495 aa, synthetic construct
SYELTQPPSVSVSPGQTARITCSGGSGSYGWYQQKPGQAPVLVIYGTYKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 84 - protein, 376 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSGGGGSEVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSS

SEQ ID NO: 85 - protein, 376 aa, synthetic construct
SYELTQPPSVSVSPGQTARITCSGGSGSYGWYQQKPGQAPVLVIYGTYKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSGGGGSEVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSS

SEQ ID NO: 86 - protein, 596 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSEVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSS

SEQ ID NO: 87 - protein, 596 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSEVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSS

SEQ ID NO: 88 - protein, 457 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 487 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 90 - protein, 485 aa, synthetic construct
EVQLVESGGGLVQPGGSLRLSCAASGFTFNTYAMNWVRQAPGKGLEWVARIRSKYNNYATYYADSVKSRTTISDDSKNTLYLQMNSLRAEDTAVYYCVRHGNFGNSYVSWFAYWGQTTVTVSSGGGGSGGGGSGGGGSGGGGSEIVVTQSPATLSVSPGERATLSCRSSTGAVTTSNYANWVQEPGQAFRGLIGGANKRAPGVPARFSGSLSGDEATLTISSLQSEDFAVYYCALWYSNLWVFGQGTKLEIKASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 91 - protein, 482 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - protein, 474 aa, synthetic construct
QVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTTLTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 93 - protein, 501 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 94 - protein, 501 aa, synthetic construct
SYELTQPPSVSVSPGQTARITCSGGSGSYGWYQQKPGQAPVLVIYGTYKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 95 - protein, 506 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 96 - protein, 506 aa, synthetic construct
SYELTQPPSVSVSPGQTARITCSGGSGSYGWYQQKPGQAPVLVIYGTYKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 97 - protein, 504 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSGGGGSEVQLVESGGGLVQPGGSLRLSCAASGFTFNTYAMNWVRQAPGKGLEWVARIRSKYNNYATYYADSVKSRTTISDDSKNTLYLQMNSLRAEDTAVYYCVRHGNFGNSYVSWFAYWGQTTVTVSSGGGGSGGGGSGGGGSGGGGSEIVVTQSPATLSVSPGERATLSCRSSTGAVTTSNYANWVQEPGQAFRGLIGGANKRAPGVPARFSGSLSGDEATLTISSLQSEDFAVYYCALWYSNLWVFGQGTKLEIK

SEQ ID NO: 98 - protein, 504 aa, synthetic construct
SYELTQPPSVSVSPGQTARITCSGGSGSYGWYQQKPGQAPVLVIYGTYKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSGGGGSEVQLVESGGGLVQPGGSLRLSCAASGFTFNTYAMNWVRQAPGKGLEWVARIRSKYNNYATYYADSVKSRTTISDDSKNTLYLQMNSLRAEDTAVYYCVRHGNFGNSYVSWFAYWGQTTVTVSSGGGGSGGGGSGGGGSGGGGSEIVVTQSPATLSVSPGERATLSCRSSTGAVTTSNYANWVQEPGQAFRGLIGGANKRAPGVPARFSGSLSGDEATLTISSLQSEDFAVYYCALWYSNLWVFGQGTKLEIK

SEQ ID NO: 99 - protein, 493 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSGGGGSQVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTTLTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 100 - protein, 493 aa, synthetic construct
SYELTQPPSVSVSPGQTARITCSGGSGSYGWYQQKPGQAPVLVIYGTYKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSGGGGSQVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTTLTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 101 - protein, 713 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSQVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTTLTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 102 - protein, 713 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSQVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTTLTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 103 - protein, 726 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 104 - protein, 726 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 105 - protein, 724 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSEVQLVESGGGLVQPGGSLRLSCAASGFTFNTYAMNWVRQAPGKGLEWVARIRSKYNNYATYYADSVKSRTTISDDSKNTLYLQMNSLRAEDTAVYYCVRHGNFGNSYVSWFAYWGQTTVTVSSGGGGSGGGGSGGGGSGGGGSEIVVTQSPATLSVSPGERATLSCRSSTGAVTTSNYANWVQEPGQAFRGLIGGANKRAPGVPARFSGSLSGDEATLTISSLQSEDFAVYYCALWYSNLWVFGQGTKLEIK

SEQ ID NO: 106 - protein, 724 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSEVQLVESGGGLVQPGGSLRLSCAASGFTFNTYAMNWVRQAPGKGLEWVARIRSKYNNYATYYADSVKSRTTISDDSKNTLYLQMNSLRAEDTAVYYCVRHGNFGNSYVSWFAYWGQTTVTVSSGGGGSGGGGSGGGGSGGGGSEIVVTQSPATLSVSPGERATLSCRSSTGAVTTSNYANWVQEPGQAFRGLIGGANKRAPGVPARFSGSLSGDEATLTISSLQSEDFAVYYCALWYSNLWVFGQGTKLEIK

SEQ ID NO: 107 - protein, 721 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 108 - protein, 721 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 109 - protein, 496 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSGGGGSEVQLLESGGGLVQPGGSLRLSCAASGFSFTGYTMNWVRQAPGKGLEWMGLINPYKGVSTYNQKVKGRFTISRDNSKNTLYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQTPGKAPKRWIYYTSRLHSGVPSRFSGSGSGTDYTFTYSSLQPEDIATYYCQQGNTLPWTFGQGTKLEIK

SEQ ID NO: 110 - protein, 496 aa, synthetic construct
SYELTQPPSVSVSPGQTARITCSGGSGSYGWYQQKPGQAPVLVIYGTYKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSGGGGSEVQLLESGGGLVQPGGSLRLSCAASGFSFTGYTMNWVRQAPGKGLEWMGLINPYKGVSTYNQKVKGRFTISRDNSKNTLYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQTPGKAPKRWIYYTSRLHSGVPSRFSGSGSGTDYTFTYSSLQPEDIATYYCQQGNTLPWTFGQGTKLEIK

SEQ ID NO: 111 - protein, 477 aa, synthetic construct
EVQLLESGGGLVQPGGSLRLSCAASGFSFTGYTMNWVRQAPGKGLEWMGLINPYKGVSTYNQKVKGRFTISRDNSKNTLYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQTPGKAPKRWIYYTSRLHSGVPSRFSGSGSGTDYTFTYSSLQPEDIATYYCQQGNTLPWTFGQGTKLEIKASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 112 - protein, 716 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSEVQLLESGGGLVQPGGSLRLSCAASGFSFTGYTMNWVRQAPGKGLEWMGLINPYKGVSTYNQKVKGRFTISRDNSKNTLYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQTPGKAPKRWIYYTSRLHSGVPSRFSGSGSGTDYTFTYSSLQPEDIATYYCQQGNTLPWTFGQGTKLEIK

SEQ ID NO: 113 - protein, 716 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGTGGGGSEVQLLESGGGLVQPGGSLRLSCAASGFSFTGYTMNWVRQAPGKGLEWMGLINPYKGVSTYNQKVKGRFTISRDNSKNTLYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQTPGKAPKRWIYYTSRLHSGVPSRFSGSGSGTDYTFTYSSLQPEDIATYYCQQGNTLPWTFGQGTKLEIK

SEQ ID NO: 114 - protein, 457 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSMGSGVSWSGYVATSIDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 495 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSMGSGVSWSGYVATSIDVWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 116 - protein, 596 aa, synthetic construct
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSSGGGGSGGGGSGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSMGSGVSWSGYVATSIDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 117 - protein, 596 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGTGGGGSEVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSS

SEQ ID NO: 118 - protein, 596 aa, synthetic construct
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSSGGGGSGGGGTGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 119 - protein, 596 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGTGGGGSEVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSS

SEQ ID NO: 120 - protein, 596 aa, synthetic construct
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTQVTVSSGGGGSGGGGTGGGGSEVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 121 - protein, 455 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 122 - protein, 215 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 123 - protein, 453 aa, synthetic construct
EVQLVESGGGLVQPGGSLRLSCAASGFTFNTYAMNWVRQAPGKGLEWVARIRSKYNNYATYYADSVKSRTTISDDSKNTLYLQMNSLRAEDTAVYYCVRHGNFGNSYVSWFAYWGQTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 124 - protein, 215 aa, synthetic construct
EIVVTQSPATLSVSPGERATLSCRSSTGAVTTSNYANWVQEPGQAFRGLIGGANKRAPGVPARFSGSLSGDEATLTISSLQSEDFAVYYCALWYSNLWVFGQGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 455 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 126 - protein, 216 aa, synthetic construct
EQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 127 - protein, 449 aa, synthetic construct
QVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 128 - protein, 214 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 452 aa, synthetic construct
EVQLLESGGGLVQPGGSLRLSCAASGFSFTGYTMNWVRQAPGKGLEWMGLINPYKGVSTYNQKVKGRFTISRDNSKNTLYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 130 - protein, 213 aa, synthetic construct
DIQMTQSPSSLSASVGDRVTITCRASQDIRNYLNWYQQTPGKAPKRWIYYTSRLHSGVPSRFSGSGSGTDYTFTYSSLQPEDIATYYCQQGNTLPWTFGQGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - skipped

(19) United States
(12) Patent Application Publication
(10) Pub. No.: US 2025/0145710 A1
(43) Pub. Date: May 8, 2025

(54) ANTIBODIES DIRECTED TO CLAUDIN 6, INCLUDING BISPECIFIC FORMATS THEREOF

(71) Applicant: Integral Molecular, Inc., Philadelphia, PA (US)

(72) Inventors: Joseph RUCKER, Philadelphia, PA (US); Kyle Doolan, Philadelphia, PA (US)

(21) Appl. No.: 18/740,950

(22) Filed: Jun. 12, 2024

Related U.S. Application Data
(62) Division of application No. 18/523,196, filed on Nov. 29, 2023, now Pat. No. 12,049,502.
(60) Provisional application No. 63/591,924, filed on Oct. 20, 2023.
(60) Provisional application No. 63/517,668, filed on Aug. 4, 2023.
(60) Provisional application No. 63/506,533, filed on Jun. 6, 2023.
(60) Provisional application No. 63/496,174, filed on Apr. 14, 2023.
(60) Provisional application No. 63/385,535, filed on Nov. 30, 2022.

(51) Int. Cl. C07K 16/28; A61K 39/00

(52) U.S. Cl. CPC C07K 16/2809; C07K 16/28; A61K 39/00; C07K 2317/31

(57) ABSTRACT

The disclosure is directed to an antibody specific for claudin 6 and CD3 and method of preparing and using the same. The present disclosure describes the isolation and characterization of antibodies, antibody fragments, and antibody variants specific for claudin 6 and CD3.

### FIELD OF THE DISCLOSURE

The disclosure is directed to compositions that bind Claudin 6, and related methods.

### BACKGROUND

Cell adhesion proteins are critical for maintaining tissue integrity, as well as regulating diverse cellular events in a wide variety of physiological and pathological processes. Among cell adhesion proteins, some members of the claudin (CLDN) family are often aberrantly expressed in various cancers. Clinical application of CLDN therapeutics has been difficult because of lack of antibody specificity for particular CLDN proteins and widespread expression of closely related CLDN family members in normal cells. Thus, there remains a significant need for improved compositions and methods that can modulate the activity of CLDN family members to treat various cancers and diseases.

### SUMMARY

Accordingly, in various aspects, the present disclosure relates to a composition that specifically binds to claudin 6 and CD3. The present disclosure describes the isolation and characterization of antibodies, antibody fragments, and antibody variants specific for claudin 6 and CD3. In some embodiments, the antibody specific for claudin 6 and CD3 is a tandem single-chain variable fragment (scFv). In some embodiments, the antibody specific for claudin 6 and CD3 is a scFv-Fab Fc antibody specific for claudin 6 and CD3. In some embodiments, the antibody specific for claudin 6 and CD3 is an IgG-(scFv)₂ antibody specific for claudin 6 and CD3. In some embodiments, the antibody specific for claudin 6 and CD3 is a scFv-Fab Fc specific for claudin 6 and CD3. In some embodiments, the antibody specific for claudin 6 and CD3 binds claudin 6 and CD3 contemporaneously.

In some embodiments, disclosed herein is a composition comprising a scFv-Fab Fc antibody specific for claudin 6 and CD3 comprising:
(a) a first heavy chain selected from:
(i) SEQ ID NO: 79,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
(ii) SEQ ID NO: 88,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
   or
(iii) SEQ ID NO: 114,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof,
(b) a second heavy chain selected from:
(i.) SEQ ID NO: 80,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof, or
(ii.) SEQ ID NO: 89,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 26, or a variant thereof, and the heavy chain CDR3 comprises an amino acid sequence of SEQ ID NO: 27, or a variant thereof, or
   a heavy chain variable region comprising heavy chain CDR1, CDR2, and CDR3 sequences, wherein the heavy chain CDR1 comprises an amino acid sequence of SEQ ID NO: 28, or a variant thereof, the heavy chain CDR2 comprises an amino acid sequence of SEQ ID NO: 29, or a variant thereof, and the heavy chain CDR3 comprises an amino acid sequence of SEQ ID NO: 30, or a variant thereof, or
(iii.) SEQ ID NO: 90,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 31, or a variant thereof, and the heavy chain CDR3 comprises an amino acid sequence of SEQ ID NO: 32, or a variant thereof, or
   a heavy chain variable region comprising heavy chain CDR1, CDR2, and CDR3 sequences, wherein the heavy chain CDR1 comprises an amino acid sequence of SEQ ID NO: 48, or a variant thereof, the heavy chain CDR2 comprises an amino acid sequence of SEQ ID NO: 34, or a variant thereof, and the heavy chain CDR3 comprises an amino acid sequence of SEQ ID NO: 35, or a variant thereof, or
(iv.) SEQ ID NO: 91,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 19, or a variant thereof, the heavy chain CDR2 comprises an amino acid sequence of SEQ ID NO: 20, or a variant thereof, and the heavy chain CDR3 comprises an amino acid sequence of ID NO: 21, or a variant thereof, or
   a heavy chain variable region comprising heavy chain CDR1, CDR2, and CDR3 sequences, wherein the heavy chain CDR1 comprises an amino acid sequence of SEQ ID NO: 49, or a variant thereof, the heavy chain CDR2 comprises an amino acid sequence of SEQ ID NO: 23, or a variant thereof, and the heavy chain CDR3 comprises an amino acid sequence of SEQ ID NO: 24, or a variant thereof, or
(v.) SEQ ID NO: 92,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 7, or a variant thereof, the heavy chain CDR2 comprises an amino acid sequence of SEQ ID NO: 36, or a variant thereof, and the heavy chain CDR3 comprises an amino acid sequence of SEQ ID NO: 9, or a variant thereof, or
   a heavy chain variable region comprising heavy chain CDR1, CDR2, and CDR3 sequences, wherein the heavy chain CDR1 comprises an amino acid sequence of SEQ ID NO: 37, or a variant thereof, the heavy chain CDR2 comprises an amino acid sequence of SEQ ID NO: 38, or a variant thereof, and the heavy chain CDR3 comprises an amino acid sequence of SEQ ID NO: 39, or a variant thereof, or
(vi.) SEQ ID NO: 111,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 40, or a variant thereof, the heavy chain CDR2 comprises an amino acid sequence of SEQ ID NO: 41, or a variant thereof, and the heavy chain CDR3 comprises an amino acid sequence of SEQ ID NO: 42, or a variant thereof, or
a heavy chain variable region comprising heavy chain CDR1, CDR2, and CDR3 sequences, wherein the heavy chain CDR1 comprises an amino acid sequence of SEQ ID NO: 43, or a variant thereof, the heavy chain CDR2 comprises an amino acid sequence of SEQ ID NO: 44, or a variant thereof, and the heavy chain CDR3 comprises an amino acid sequence of SEQ ID NO: 45, or a variant thereof,
and
(c) a first light chain selected from:
(i) SEQ ID NO: 67,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of ID NO: 3, or a variant thereof,
or
(iii) SEQ ID NO: 65,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

In some embodiments, the Fc is from IgG.

In some embodiments, the IgG is human IgG. In some embodiments, the human IgG is selected from IgG1, IgG2, IgG3, and IgG4.

In some embodiments, the antibody is formed through a knob-in-hole interaction in the Fc region. In some embodiments, the human IgG Fc comprises one or mutations to promote knob-in-hole interaction. In some embodiments, the mutations are selected from (i) T366Y or T366W, and (ii) Y407T, Y407A, or Y407V. In some embodiments, the mutations are selected from:
(a) T366Y and Y407T or T366Y/F405A and T394W/Y407T in human IgG1, or
(b) T366W/D399C and T366S/L368A/K392C/Y407V; T366W/K392C and T366S/L368A/D399C/Y407V; S354C/T366W and Y349C/T366S/L368A/Y407V; Y349C/T366W and S354C/T366S/L368A/Y407V; E356C/T366W and Y349C/T366S/L368A/Y407V; Y349C/T366W and E356C/T366S/L368A/Y407V; E357C/T366W and Y349C/T366S/L368A/Y407V; or Y349C/T366W and E357C/T366S/L368A/Y407V,
in human IgG1.

In some embodiments, the human IgG Fc comprises one or mutations to reduce or eliminate the effector function of the Fc domains. In some embodiments, the mutations are L234A and L235A (LALA) substitutions in human IgG1.

In some embodiments, the human IgG Fc comprises one or mutations to stabilize a hinge region in the Fc domain. In some embodiments, the mutation is S228P.

In some embodiments, disclosed herein is a composition comprising a scFv-Fab Fe antibody specific for claudin 6 and CD3 comprising:
(a) a first heavy chain selected from:
(i) SEQ ID NO: 79,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(ii) SEQ ID NO: 88,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
   or
(iii) SEQ ID NO: 114,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof,
(b) a second heavy chain selected from:
SEQ ID NO: 81,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
and
(c) a light chain selected from:
(i) SEQ ID NO: 67,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
or
(iii) SEQ ID NO: 65,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

In some embodiments, the Fc is from IgG. In some embodiments, the IgG is human IgG. In some embodiments, the human IgG is selected from IgG1, IgG2, IgG3, and IgG4.

In some embodiments, the antibody is formed through a knob-in-hole interaction in the Fc region. In some embodiments, the human IgG Fc comprises one or mutations to promote knob-in-hole interaction. In some embodiments, the mutations are selected from (i) T366Y or T366W, and (ii) Y407T, Y407A, or Y407V. In some embodiments, mutations are:
(a) T366Y and Y407T or T366Y/F405A and T394W/Y407T in human IgG1 or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

In some embodiments, the human IgG Fc comprises one or mutations to reduce or eliminate the effector function of the Fc domains. In some embodiments, the mutations are L234A and L235A (LALA) substitutions in human IgG1. In some embodiments, the human IgG Fc comprises one or mutations to stabilize a hinge region in the Fc domain. In some embodiments, mutation is S228P.

In some embodiments, disclosed herein is a composition comprising a scFv-Fab Fc antibody specific for claudin 6 and CD3 comprising:
(a) a first heavy chain selected from:
(i) SEQ ID NO: 79,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(ii) SEQ ID NO: 88,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
   or
(iii) SEQ ID NO: 114,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof,
(b) a second heavy chain selected from:
(i.) SEQ ID NO: 117,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
(ii.) SEQ ID NO: 119,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
(iii.) SEQ ID NO: 118,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(iv.) SEQ ID NO: 120,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
and
(c) a light chain selected from:
(i) SEQ ID NO: 67,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
or
(iii) SEQ ID NO: 65, an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

In some embodiments, the Fc is from IgG. In some embodiments, the IgG is human IgG. In some embodiments, the human IgG is selected from IgG1, IgG2, IgG3, and IgG4.

In some embodiments, the antibody is formed through a knob-in-hole interaction in the Fc region. In some embodiments, the human IgG Fc comprises one or mutations to promote knob-in-hole interaction. In some embodiments, the mutations are selected from (i) T366Y or T366W, and (ii) Y407T, Y407A, or Y407V. In some embodiments, the mutations are:
(a) T366Y and Y407T or T366Y/F405A and T394W/Y407T in human IgG1 or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

In some embodiments, the human IgG Fc comprises one or mutations to reduce or eliminate the effector function of the Fc domains. In some embodiments, the mutations are L234A and L235A (LALA) substitutions in human IgG1. In some embodiments, the human IgG Fc comprises one or mutations to stabilize a hinge region in the Fc domain. In some embodiments, the mutation is S228P.

In some embodiments, disclosed herein is a composition comprising a scFv-Fab Fc antibody specific for claudin 6 and CD3 comprising:
(a) a first heavy chain selected from:
(i) SEQ ID NO: 79,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
   or
(ii) SEQ ID NO: 88,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
   or
(iii) SEQ ID NO: 114,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof,
(b) a second heavy chain selected from:
(i) SEQ ID NO: 121,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 26, or a variant thereof, and the heavy chain CDR3 comprises an amino acid sequence of SEQ ID NO: 27, or a variant thereof,
(ii) SEQ ID NO: 123,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 31, or a variant thereof, and the heavy chain CDR3 comprises an amino acid sequence of SEQ ID NO: 32, or a variant thereof,
(iii) SEQ ID NO: 125,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 19, or a variant thereof, the heavy chain CDR2 comprises an amino acid sequence of SEQ ID NO: 20, or a variant thereof, and the heavy chain CDR3 comprises an amino acid sequence of SEQ ID NO: 21, or a variant thereof,
(iv) SEQ ID NO: 127,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 7, or a variant thereof, the heavy chain CDR2 comprises an amino acid sequence of SEQ ID NO: 36, or a variant thereof, and the heavy chain CDR3 comprises an amino acid sequence of SEQ ID NO: 9, or a variant thereof, or
(v) SEQ ID NO: 129,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 40, or a variant thereof, the heavy chain CDR2 comprises an amino acid sequence of SEQ ID NO: 41, or a variant thereof, and the heavy chain CDR3 comprises an amino acid sequence of SEQ ID NO: 42, or a variant thereof, and
(c) a first light chain selected from:
(i) SEQ ID NO: 67,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
or
(iii) SEQ ID NO: 65, an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
and
(d) a second light chain selected from:
(i) SEQ ID NO: 122,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 28, or a variant thereof, the light chain CDR2 comprises an amino acid sequence of SEQ ID NO: 29, or a variant thereof, and the light chain CDR3 comprises an amino acid sequence of SEQ ID NO: 30, or a variant thereof, or
(ii) SEQ ID NO: 124,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 33, or a variant thereof, the light chain CDR2 comprises an amino acid sequence of SEQ ID NO: 34, or a variant thereof, and the light chain CDR3 comprises an amino acid sequence of SEQ ID NO: 35, or a variant thereof, or
(iii) SEQ ID NO: 126,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of ID NO: 22, or a variant thereof, the light chain CDR2 comprises an amino acid sequence of SEQ ID NO: 23, or a variant thereof, and the light chain CDR3 comprises an amino acid sequence of SEQ ID NO: 24, or a variant thereof, or
(iv) SEQ ID NO: 128,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 37, or a variant thereof, the light chain CDR2 comprises an amino acid sequence of SEQ ID NO: 38, or a variant thereof, and the light chain CDR3 comprises an amino acid sequence of SEQ ID NO: 39, or a variant thereof, or
(v) SEQ ID NO: 130,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 43, or a variant thereof, the light chain CDR2 comprises an amino acid sequence of SEQ ID NO: 44, or a variant thereof, and the light chain CDR3 comprises an amino acid sequence of SEQ ID NO: 45, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

In some embodiments, disclosed herein is a bispecific antibody, wherein the bispecific antibody comprises three polypeptides (e.g., a first polypeptide, a second polypeptide, and a third polypeptide) that form a first antigen binding domain that binds to CLDN6, and a second antigen binding domain that binds to CD3. In some embodiments, the first polypeptide comprises a first light chain comprising a first variable light chain region (first V_{L}), wherein the first variable light chain region comprises a CDR1 sequence comprising the amino acid sequence of SEQ ID NO: 1, a CDR2 sequence comprising the amino acid sequence of SEQ ID NO: 2, and a CDR3 sequence comprising the amino acid sequence of SEQ ID NO: 3. In some embodiments, the second polypeptide comprises a first heavy chain comprising a first variable region heavy chain region (first VH), wherein the first variable heavy chain region comprises a CDR1 sequence comprising the amino acid sequence of SEQ ID NO: 4, a CDR2 sequence comprising the amino acid sequence of SEQ ID NO: 5, and a CDR3 sequence comprising the amino acid sequence of SEQ ID NO: 6. In some embodiments, the third polypeptide comprises a second heavy chain and a second a light chain, wherein the second heavy chain comprises a second variable heavy chain region (second VH) comprising a CDR1 sequence comprising the amino acid sequence of SEQ ID NO: 25, a CDR2 sequence comprising the amino acid sequence of SEQ ID NO: 26, and a CDR3 sequence comprising the amino acid sequence of SEQ ID NO: 27; wherein the second light chain comprises a second variable light chain region (second V_{L}) comprising a CDR1 sequence comprising the amino sequence of SEQ ID NO: 28, a CDR2 sequence comprising the amino acid sequence of SEQ ID NO: 29, and a CDR3 sequence comprising the amino acid sequence of SEQ ID NO: 30. In some embodiments, the second heavy chain and the second light chain are linked by a peptide linker. In some embodiments, the peptide linker is as described herein. In some embodiments, the peptide linker comprises the amino acid sequence of (GKPGSGKPGSGKPGSGKPGS) SEQ ID NO: 53. In some embodiments, the peptide linker comprising one or more glycines and serines is replaced with another peptide linker or functionally equivalent variation thereof. In some embodiments, the first V_{L} and the first VH interact to form the antigen binding domain that binds to CLDN6. In some embodiments, the second V_{L} and the second VH interact to form the antigen binding domain that binds to CD3. In some embodiments, the second V_{L} and the second VH are in a scFv format. In some embodiments, the first V_{L} and the first VH are in a Fab format, or a fragment thereof.

In some embodiments, the first variable light chain region of the first polypeptide comprises the amino acid sequence of SEQ ID NO: 68, or an amino acid sequence having at least about 90%, or about 93%, or about 95%, or about 97%, or about 98%, or about 99% identity thereto.

In some embodiments, the first variable heavy chain region of the second polypeptide comprises the amino acid sequence of SEQ ID NO: 69, or an amino acid sequence having at least about 90%, or about 93%, or about 95%, or about 97%, or about 98%, or about 99% identity thereto.

In some embodiments, the second variable heavy chain region of the third polypeptide comprises the amino acid sequence of SEQ ID NO: 70, or an amino acid sequence having at least about 90%, or about 93%, or about 95%, or about 97%, or about 98%, or about 99% identity thereto.

In some embodiments, the second variable light chain region of the third polypeptide comprises the amino acid sequence of SEQ ID NO: 71, or an amino acid sequence having at least about 90%, or about 93%, or about 95%, or about 97%, or about 98%, or about 99% identity thereto.

In some embodiments, the first polypeptide compromising the light chain comprises the first variable light chain region and a light chain constant domain, which can be referred to as the first light chain constant domain. In some embodiments, the first light chain constant domain comprises the amino acid sequence of SEQ ID NO: 72, or an amino acid sequence having at least about 90%, or about 93%, or about 95%, or about 97%, or about 98%, or about 99% identity thereto.

In some embodiments, the first polypeptide comprises the amino acid sequence of SEQ ID NO: 67, or an amino acid sequence having at least about 90%, or about 93%, or about 95%, or about 97%, or about 98%, or about 99% identity thereto.

In some embodiments, the second polypeptide compromising the first heavy chain comprises the first variable heavy chain region and a heavy chain constant domain, which can be referred to as the first heavy chain constant domain. In some embodiments, the first heavy chain constant domain comprises the amino acid sequence of SEQ ID NO: 73, or an amino acid sequence having at least about 90%, or about 93%, or about 95%, or about 97%, or about 98%, or about 99% identity thereto.

In some embodiments, the second polypeptide comprises the amino acid sequence of SEQ ID NO: 79, or an amino acid sequence having at least about 90%, or about 93%, or about 95%, or about 97%, or about 98%, or about 99% identity thereto.

In some embodiments, the third polypeptide comprises a constant domain. In some embodiments, the constant domain is linked to the C-terminus of the second variable light chain region. In some embodiments, there is no peptide linker between the C-terminus of the second variable light chain region and the constant domain. In some embodiments, the constant domain present in the third polypeptide comprises the amino acid sequence of SEQ ID NO: 74, or an amino acid sequence having at least about 90%, or about 93%, or about 95%, or about 97%, or about 98%, or about 99% identity thereto.

In some embodiments, the third polypeptide comprises the amino acid sequence of SEQ ID NO: 89, or an amino acid sequence having at least about 90%, or about 93%, or about 95%, or about 97%, or about 98%, or about 99% identity thereto.

In some embodiments, the first polypeptide comprises the amino acid sequence of SEQ ID NO: 67; the second polypeptide comprises the amino acid sequence of SEQ ID NO: 79; and the third polypeptide comprises the amino acid sequence of SEQ ID NO: 89.

As used herein, the term constant domain refers to the Fc domain. The constant domains exemplified above are optional embodiments and other constant domains can be substituted for the constant domains described herein.

In some embodiments, the Fc is from IgG. In some embodiments, the IgG is human IgG. In some embodiments, the human IgG is selected from IgG1, IgG2, IgG3, and IgG4.

In some embodiments, the antibody is formed through a knob-in-hole interaction in the Fc region. In some embodiments, the human IgG Fc comprises one or mutations to promote knob-in-hole interaction. In some embodiments, the mutations are selected from (i) T366Y or T366W, and (ii) Y407T, Y407A, or Y407V. In some embodiments, the mutations are:
(a) T366Y and Y407T or T366Y/F405A and T394W/Y407T in human IgG1 or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

In some embodiments, the human IgG Fc comprises one or mutations to reduce or eliminate the effector function of the Fc domains. In some embodiments, the mutations are L234A and L235A (LALA) substitutions in human IgG1. In some embodiments, the human IgG Fc comprises one or mutations to stabilize a hinge region in the Fc domain. In some embodiments, the mutation is S228P.

In some embodiments, the composition binds claudin 6 and CD3 contemporaneously.

In some embodiments, the composition binds claudin 6 with an affinity of less than 10 nM and with at least 100 fold greater affinity than claudin 9, claudin 3, and/or claudin 4.

In some embodiments, disclosed herein is a pharmaceutical composition comprising an isolated antibody of any one of the preceding embodiments, or a nucleic acid molecule encoding the same. In some embodiments, the composition is an injectable pharmaceutical composition. In some embodiments, the composition is sterile. In some embodiments, the composition is pyrogen free.

In some embodiments, disclosed herein is a nucleic acid molecule encoding an antibody or an amino acid sequence of any of the preceding embodiments.

In some embodiments, disclosed herein is a vector comprising the nucleic acid molecule of any of the preceding embodiments.

In some embodiments, disclosed herein is a cell comprising the nucleic acid molecule of any of the preceding embodiments, or the vector of any of the preceding embodiments.

In some embodiments, disclosed herein is a method for modulating and/or targeting claudin 6 and CD3 in a biological cell, comprising contacting the cell with a composition of any of the preceding embodiments.

In some embodiments, disclosed herein is a method for modulating claudin 6 activity in a biological cell comprising contacting a cell expressing claudin 6 with a composition of any of the preceding embodiments.

In some embodiments, disclosed herein is a method for inhibiting the function of claudin 6 in a biological cell comprising contacting a cell expressing claudin 6 with a composition of any of the preceding embodiments.

In some embodiments, disclosed herein is a method for treating or preventing cancer comprising administering an effective amount of the composition of any of the preceding embodiments to a subject in need thereof.

In some embodiments, disclosed herein is a use of the composition of any of the preceding embodiments for the preparation of a medicament for the treatment of prevention of cancer.

In some embodiments, disclosed herein is a method or use of any one of the preceding embodiments, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

In some embodiments, disclosed herein is an isolated antibody comprising one or more of the sequences disclosed herein.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE DRAWINGS

FIG. 1 shows images of illustrative bispecific formats, including a tandem scFv (left), a scFv-Fab IgG (middle), and an IgG-(scFv)₂ (right).

FIG. 2 is an image showing a schematic of the structure of CLDN6 and illustrating some of the challenges in developing an antibody specific for CLDN6.

FIG. 3 is a graph of RNA sequencing data showing how claudin 6 is overexpressed in multiple cancers, including ovarian cancer, lung cancer, endometrial cancer, and gastric cancer.

FIG. 4 is an image showing a non-limiting monospecificity challenge in developing antibodies specific to claudin 6, as the antibody needs to discriminate between a single amino acid side chain at residue 156 on CLDN6 and CLDN9.

FIG. 5 is a chart showing an overview of the data related to the four selected antibodies.

FIG. 6 shows graphs and images of quality control data from the IMC-16-3 scFv-Fab IgG antibody (SEQ ID NOs: 79, 67, and 89).

FIG. 7 shows graphs and images of quality control data from the IMC-16-15 scFv-Fab IgG antibody (SEQ ID NOs: 114, 65, and 89).

FIG. 8 shows graphs and images of quality control data from the IMC-21-1 IgG-(scFv)₂ antibody (SEQ ID NOs: 86, and 67).

FIG. 9 shows graphs and images of quality control data from the IMC-2-7 tandem scFv antibody (SEQ ID NO: 95).

FIG. 10 shows two graphs of target binding data from flow cytometry experiments. The left panel in FIG. 10 shows target binding data of the IMC-16-3 scFv-Fab IgG antibody (circles), IMC-16-15 scFv-Fab IgG antibody (squares), IMC-21-1 IgG-(scFv)₂ antibody (triangles), and the IMC-2-7 tandem scFv antibody (inverted triangles) to human CLDN6. The right panel in FIG. 10 shows target binding data of the IMC-16-3 scFv-Fab IgG antibody (circles), IMC-16-15 scFv-Fab IgG antibody (squares), IMC-21-1 IgG-(scFv)₂ antibody (triangles), and the IMC-2-7 tandem scFv antibody (inverted triangles) to human CD3. The table at the bottom of FIG. 10 shows binding EC50 data for molecules against CLDN6 and CD3.

FIG. 11A, FIG. 11B, FIG. 11C, and FIG. 11D are graphs showing parental and benchmark antibody binding data from flow cytometry experiments. FIG. 11A is a graph showing binding of IM271-1HEP antibody to human CLDN6 (circle shapes), human CLDN4 (square shapes), human CLDN3 (triangle shapes), human CLDN9 (inverted triangle shapes), and a negative control (diamond shapes). FIG. 11B is a graph showing binding of IM271-1HHP antibody to human CLDN6 (circle shapes), human CLDN4 (square shapes), human CLDN3 (triangle shapes), human CLDN9 (inverted triangle shapes), and a negative control (diamond shapes). FIG. 11C is a graph showing binding of IM271-1HFJ antibody to human CLDN6 (circle shapes), human CLDN4 (square shapes), human CLDN3 (triangle shapes), human CLDN9 (inverted triangle shapes), and a negative control (diamond shapes). FIG. 11D is a graph showing binding of an antibody to human CLDN6 (circle shapes), human CLDN4 (square shapes), human CLDN3 (triangle shapes), human CLDN9 (inverted triangle shapes), and a negative control (diamond shapes).

FIG. 12A, FIG. 12B, FIG. 12C, and FIG. 12D are graphs showing target binding data from flow cytometry experiments. FIG. 12A is a graph showing binding of the IMC-2-7 tandem scFv antibody to human CLDN6 (circle shapes), human CLDN4 (square shapes), human CLDN3 (triangle shapes), human CLDN9 (inverted triangle shapes), and a negative control (diamond shapes). FIG. 12B is a graph showing binding of the IMC-16-3 scFv-Fab IgG antibody to human CLDN6 (circle shapes), human CLDN4 (square shapes), human CLDN3 (triangle shapes), human CLDN9 (inverted triangle shapes), and a negative control (diamond shapes). FIG. 12C is a graph showing binding of the IMC-16-15 scFv-Fab IgG antibody to human CLDN6 (circle shapes), human CLDN4 (square shapes), human CLDN3 (triangle shapes), human CLDN9 (inverted triangle shapes), and a negative control (diamond shapes). FIG. 12D is a graph showing binding of the IMC-21-1 IgG-(scFv)₂ antibody to human CLDN6 (circle shapes), human CLDN4 (square shapes), human CLDN3 (triangle shapes), human CLDN9 (inverted triangle shapes), and a negative control (diamond shapes).

FIG. 13 shows two graphs of cellular toxicity data from T-cell dependent cellular cytotoxicity experiments. The left panel in FIG. 13 shows cytotoxicity data of the IMC-16-3 scFv-Fab IgG antibody, IMC-16-15 scFv-Fab IgG antibody, IMC-21-1 IgG-(scFv)₂ antibody, IMC-2-7 tandem scFv antibody, and the IMC-16-C2 antibody (negative control) in ovarian cancer OV-90 cells. The right panel in FIG. 13 shows target binding data of the IMC-16-3 scFv-Fab IgG antibody, IMC-16-15 scFv-Fab IgG antibody, IMC-21-1 IgG-(scFv)₂ antibody, IMC-2-7 tandem scFv antibody, and the IMC-16-C2 antibody in HEK cells.

FIG. 14A, FIG. 14B, FIG. 14C, FIG. 14D, and FIG. 14E are graphs showing the production of cytokines by human PBMCs co-cultured with CLDN6 positive OV-90 cells in the presence or absence of the IMC-16-3 scFv-Fab IgG antibody. FIG. 14A illustrates the results for IL-2 production, FIG. 14B illustrates the results for IL-6 production, FIG. 14C illustrates the results for IL-10 production, FIG. 14D illustrates the results for IFN-7 production, and FIG. 14E illustrates the results for TNF-α production.

FIG. 15A, FIG. 15B, FIG. 15C, FIG. 15D, and FIG. 15E are graphs showing the production of cytokines by human PBMCs co-cultured with CLDN6 positive OV-90 cells in the presence or absence of the IMC-16-15 scFv-Fab IgG. FIG. 15A illustrates the results for IL-2 production, FIG. 15B illustrates the results for IL-6 production, FIG. 15C illustrates the results for IL-10 production, FIG. 15D illustrates the results for IFN-γ production, and FIG. 15E illustrates the results for TNF-α production.

FIG. 16A, FIG. 16B, FIG. 16C, FIG. 16D, and FIG. 16E are graphs showing the production of cytokines by human PBMCs co-cultured with CLDN6 positive OV-90 cells in the presence or absence of the IMC-21-1 IgG-(scFv)₂ antibody. FIG. 16A illustrates the results for IL-2 production, FIG. 16B illustrates the results for IL-6 production, FIG. 16C illustrates the results for IL-10 production, FIG. 16D illustrates the results for IFN-γ production, and FIG. 16E illustrates the results for TNF-α production.

FIG. 17A, FIG. 17B, FIG. 17C, FIG. 17D, and FIG. 17E are graphs showing the production of cytokines by human PBMCs co-cultured with CLDN6 positive OV-90 cells in the presence or absence of the IMC-2-7 tandem scFv antibody. FIG. 17A illustrates the results for IL-2 in production, FIG. 17B illustrates the results for IL-6 in production, FIG. 17C illustrates the results for IL-10 in production, FIG. 17D illustrates the results for IFN-γ in production, and FIG. 17E illustrates the results for TNF-α in production.

FIG. 18 shows a graph of cellular toxicity data from T-cell dependent cellular cytotoxicity experiments. FIG. 18 shows cytotoxicity data of the IMC-2-7 tandem scFv antibody, IMC-16-3 scFv-Fab IgG antibody, IMC-16-15 scFv-Fab IgG antibody, IMC-21-1 IgG-(scFv)₂ antibody, and a negative control (CKV063 LALA) in K562 cells. The table to the right of FIG. 18 shows EC50 values for cellular toxicity.

FIG. 19A, FIG. 19B, FIG. 19C, FIG. 19D, FIG. 19E, FIG. 19F, FIG. 19G, and FIG. 19H are graphs showing cellular toxicity and IFN-γ release potency data at 24 hours and 48 hours from cellular cytotoxicity experiments. FIG. 19A shows cellular toxicity and IFN-7 release data of the IMC-16-3 scFv-Fab IgG antibody at 24 hours. FIG. 19B shows cellular toxicity and IFN-γ release data of the IMC-16-15 scFv-Fab IgG antibody at 24 hours. FIG. 19C shows cellular toxicity and IFN-γ release data of the IMC-21-1 IgG-(scFv)₂ antibody at 24 hours. FIG. 19D shows cellular toxicity and IFN-γ release data of the IMC-2-7 tandem scFv antibody at 24 hours. FIG. 19E shows cellular toxicity and IFN-γ release data of the IMC-16-3 scFv-Fab IgG antibody at 48 hours. FIG. 19F shows cellular toxicity and IFN-γ release data of the IMC-16-15 scFv-Fab IgG antibody at 48 hours. FIG. 19G shows cellular toxicity and IFN-γ release data of the IMC-21-1 IgG-(scFv)₂ antibody at 48 hours. FIG. 19H shows cellular toxicity and IFN-γ release data of the IMC-2-7 tandem scFv antibody at 48 hours. The CKV063 LALA antibody is a negative control in each experiment.

FIG. 20A, FIG. 20B, FIG. 20C, FIG. 20D, FIG. 20E, FIG. 20F, FIG. 20G, FIG. 20H, FIG. 20I, and FIG. 20J are graphs showing cellular toxicity and IFN-γ release potency data at 24 hours and 48 hours from cellular cytotoxicity experiments. FIG. 20A shows cellular toxicity and IFN-γ release data of the IMC-16-7 antibody (SEQ ID NOs: 79, 67, and 91) at 24 hours. FIG. 20B shows cellular toxicity and IFN-γ release data of the IMC-2-7 tandem scFv antibody at 24 hours. FIG. 20C shows cellular toxicity and IFN-γ release data of the IMC-16-3 scFv-Fab IgG antibody at 24 hours. FIG. 20D shows cellular toxicity and IFN-γ release data of the IMC-16-15 scFv-Fab IgG antibody at 24 hours. FIG. 20E shows cellular toxicity and IFN-7 release data of the IMC-21-1 IgG-(scFv)₂ antibody at 24 hours. FIG. 20F shows cellular toxicity and IFN-γ release data of the IMC-16-7 antibody at 48 hours. FIG. 20G shows cellular toxicity and IFN-γ release data of the IMC-2-7 tandem scFv antibody at 48 hours. FIG. 20H shows cellular toxicity and IFN-γ release data of the IMC-16-3 scFv-Fab IgG antibody at 48 hours. FIG. 20I shows cellular toxicity and IFN-γ release data of the IMC-16-15 scFv-Fab IgG antibody at 48 hours. FIG. 20J shows cellular toxicity and IFN-γ release data of the IMC-21-1 IgG-(scFv)₂ antibody at 48 hours. The CKV063 LALA antibody is a negative control in each experiment.

FIG. 21A, FIG. 21B, FIG. 21C, FIG. 21D, FIG. 21E, FIG. 21F, FIG. 21G, FIG. 21H, FIG. 21I, and FIG. 21J are graphs showing cellular toxicity and IL-2 release potency data at 24 hours and 48 hours from cellular cytotoxicity experiments. FIG. 21A shows cellular toxicity and IL-2 release data of the IMC-16-7 antibody (SEQ ID NOs: 79, 67, and 91) at 24 hours. FIG. 21B shows cellular toxicity and IL-2 release data of the IMC-2-7 tandem scFv antibody at 24 hours. FIG. 21C shows cellular toxicity and IL-2 release data of the IMC-16-3 scFv-Fab IgG antibody at 24 hours. FIG. 21D shows cellular toxicity and IL-2 release data of the IMC-16-15 scFv-Fab IgG antibody at 24 hours. FIG. 21E shows cellular toxicity and IL-2 release data of the MC-21-1 antibody at 24 hours. FIG. 21F shows cellular toxicity and IL-2 release data of the IMC-16-7 antibody at 48 hours. FIG. 21G shows cellular toxicity and IL-2 release data of the IMC-2-7 tandem scFv antibody at 48 hours. FIG. 21H shows cellular toxicity and IL-2 release data of the IMC-16-3 scFv-Fab IgG antibody at 48 hours. FIG. 21I shows cellular toxicity and IL-2 release data of the IMC-16-15 scFv-Fab IgG antibody at 48 hours. FIG. 21J shows cellular toxicity and IL-2 release data of the IMC-21-1 IgG-(scFv)₂ antibody at 48 hours. The CKV063 LALA antibody is a negative control in each experiment.

FIG. 22A, FIG. 22B, FIG. 22C, FIG. 22D, FIG. 22E, FIG. 22F, FIG. 22G, FIG. 22H, FIG. 22I, and FIG. 22J are graphs showing cellular toxicity and IL-10 release potency data at 24 hours and 48 hours from cytotoxicity experiments. FIG. 22A shows cellular toxicity and IL-10 release data of the IMC-16-7 antibody at 24 hours. FIG. 22B shows cellular toxicity and IL-10 release data of the IMC-2-7 tandem scFv antibody at 24 hours. FIG. 22C shows cellular toxicity and IL-10 release data of the IMC-16-3 scFv-Fab IgG antibody at 24 hours. FIG. 22D shows cellular toxicity and IL-10 release data of the IMC-16-15 scFv-Fab IgG antibody at 24 hours. FIG. 22E shows cellular toxicity and IL-10 release data of the IMC-21-1 IgG-(scFv)₂ antibody at 24 hours. FIG. 22F shows cellular toxicity and IL-10 release data of the IMC-16-7 antibody at 48 hours. FIG. 22G shows cellular toxicity and IL-10 release data of the IMC-2-7 tandem scFv antibody at 48 hours. FIG. 22H shows cellular toxicity and IL-10 release data of the IMC-16-3 scFv-Fab IgG antibody at 48 hours. FIG. 22I shows cellular toxicity and IL-10 release data of the IMC-16-15 scFv-Fab IgG antibody at 48 hours. FIG. 22J shows cellular toxicity and IL-10 release data of the IMC-21-1 IgG-(scFv)₂ antibody at 48 hours. The CKV063 LALA antibody is a negative control in each experiment.

FIG. 23A, FIG. 23B, FIG. 23C, FIG. 23D, FIG. 23E, FIG. 23F, FIG. 23G, FIG. 23H, FIG. 23I, and FIG. 23J are graphs showing cellular toxicity and TNF-α release potency data at 24 hours and 48 hours from cytotoxicity experiments. FIG. 23A shows cellular toxicity and TNF-α release data of the IMC-16-7 antibody at 24 hours. FIG. 23B shows cellular toxicity and TNF-α release data of the IMC-2-7 tandem scFv antibody at 24 hours. FIG. 23C shows cellular toxicity and TNF-α release data of the IMC-16-3 scFv-Fab IgG antibody at 24 hours. FIG. 23D shows cellular toxicity and TNF-α release data of the IMC-16-15 scFv-Fab IgG antibody at 24 hours. FIG. 23E shows cellular toxicity and TNF-α release data of the IMC-21-1 IgG-(scFv)₂ antibody at 24 hours. FIG. 23F shows cellular toxicity and TNF-α release data of the IMC-16-7 antibody at 48 hours. FIG. 23G shows cellular toxicity and TNF-α release data of the IMC-2-7 tandem scFv antibody at 48 hours. FIG. 23H shows cellular toxicity and TNF-α release data of the IMC-16-3 scFv-Fab IgG antibody at 48 hours. FIG. 23I shows cellular toxicity and TNF-α release data of the IMC-16-15 scFv-Fab IgG antibody at 48 hours. FIG. 23J shows cellular toxicity and TNF-α release data of the IMC-21-1 IgG-(scFv)₂ antibody at 48 hours. The CKV063 LALA antibody is a negative control in each experiment.

FIG. 24A, FIG. 24B, FIG. 24C, FIG. 24D, FIG. 24E, FIG. 24F, FIG. 24G, and FIG. 24H are graphs showing cytokine analysis of IFN-γ and IL-2 at 24 hours and 48 hours. FIG. 24A is a graph showing IFN-γ levels at 24 hours and 48 hours for the IMC-2-7 tandem scFv antibody. FIG. 24B is a graph showing IFN-γ levels at 24 hours and 48 hours for the IMC-16-3 scFv-Fab IgG antibody. FIG. 24C is a graph showing IFN-γ levels at 24 hours and 48 hours for the IMC-16-15 scFv-Fab IgG antibody. FIG. 24D is a graph showing IFN-γ levels at 24 hours and 48 hours for the IMC-21-1 IgG-(scFv)₂ antibody. FIG. 24E is a graph showing IL-2 levels at 24 hours and 48 hours for the IMC-2-7 tandem scFv antibody. FIG. 24F is a graph showing IL-2 levels at 24 hours and 48 hours for the IMC-16-3 scFv-Fab IgG antibody. FIG. 24G is a graph showing IL-2 levels at 24 hours and 48 hours for the IMC-16-15 scFv-Fab IgG antibody. FIG. 24H is a graph showing IL-2 levels at 24 hours and 48 hours for the IMC-21-1 IgG-(scFv)₂ antibody.

FIG. 25A, FIG. 25B, FIG. 25C, FIG. 25D, FIG. 25E, FIG. 25F, FIG. 25G, FIG. 25H, FIG. 25I, and FIG. 25J are graphs showing cytokine analysis of IL-10 and TNF-α at 24 hours and 48 hours. FIG. 25A is a graph showing IL-10 levels at 24 hours and 48 hours for the IMC-16-7 antibody. FIG. 25B is a graph showing IL-10 levels at 24 hours and 48 hours for the IMC-2-7 tandem scFv antibody. FIG. 25C is a graph showing IL-10 levels at 24 hours and 48 hours for the IMC-16-3 scFv-Fab IgG antibody. FIG. 25D is a graph showing IL-10 levels at 24 hours and 48 hours for the IMC-16-15 scFv-Fab IgG antibody. FIG. 25E is a graph showing IL-10 levels at 24 hours and 48 hours for the IMC-21-1 IgG-(scFv)₂ antibody. FIG. 25F is a graph showing TNF-α levels at 24 hours and 48 hours for the IMC-16-7 antibody. FIG. 25G is a graph showing TNF-α levels at 24 hours and 48 hours for the IMC-2-7 tandem scFv antibody. FIG. 25H is a graph showing TNF-α levels at 24 hours and 48 hours for the IMC-16-3 scFv-Fab IgG antibody. FIG. 25I is a graph showing TNF-α levels at 24 hours and 48 hours for the IMC-16-15 scFv-Fab IgG antibody. FIG. 25J is a graph showing TNF-α levels at 24 hours and 48 hours for the IMC-21-1 IgG-(scFv)₂ antibody.

FIG. 26A and FIG. 26B are graphs showing the production of TNF-α by human PBMCs co-cultured with either claudin 6 expressing HEK cells or OV-90 cells. FIG. 26A shows antibody induced TNF-α levels for the IMC-16-13 scFv-Fab IgG antibody (SEQ ID NOs: 114, 65, and 80), IMC-16-15 scFv-Fab IgG antibody, IMC-16-3 scFv-Fab IgG antibody, IMC-2-7 tandem scFv antibody, IMC-2-13 tandem scFv antibody (SEQ ID NO: 115), IMC-20-13 IgG-scFv antibody (SEQ ID NOs: 114, 65, and 116), IMC-21-1 IgG-(scFv)2 antibody, IMC-21-6 IgG-(scFv)2 antibody (SEQ ID NOs: 104 and 66), and the IMC-2-3 tandem scFv antibody (SEQ ID NO: 84) in the PBMC:HEK-CLDN6 co-culture. FIG. 26B shows antibody induced TNF-α levels for the IMC-16-13 scFv-Fab IgG antibody, IMC-16-15 scFv-Fab IgG antibody, IMC-16-3 scFv-Fab IgG antibody, IMC-2-7 tandem scFv antibody, IMC-2-13 tandem scFv antibody, IMC-20-13 IgG-scFv antibody, IMC-21-1 IgG-(scFv)2 antibody, IMC-21-6 IgG-(scFv)2 antibody, and the IMC-2-3 tandem scFv antibody in the PBMC:OV-90 co-culture.

FIG. 27A and FIG. 27B are graphs showing the production of IFN-γ by human PBMCs co-cultured with either claudin 6 expressing HEK cells or OV-90 cells. FIG. 27A shows antibody induced IFN-γ levels for the IMC-16-13 scFv-Fab IgG antibody, IMC-16-15 scFv-Fab IgG antibody, IMC-16-3 scFv-Fab IgG antibody, IMC-2-7 tandem scFv antibody, IMC-2-13 tandem scFv antibody, IMC-20-13 IgG-scFv antibody, IMC-21-1 IgG-(scFv)2 antibody, IMC-21-6 IgG-(scFv)2 antibody, and the IMC-2-3 tandem scFv antibody in the PBMC:HEK-CLDN6 co-culture. FIG. 27B shows antibody induced IFN-γ levels for the IMC-16-13 scFv-Fab IgG antibody, IMC-16-15 scFv-Fab IgG antibody, IMC-16-3 scFv-Fab IgG antibody, IMC-2-7 tandem scFv antibody, IMC-2-13 tandem scFv antibody, IMC-20-13 IgG-scFv antibody, IMC-21-1 IgG-(scFv)2 antibody, IMC-21-6 IgG-(scFv)2 antibody, and the IMC-2-3 tandem scFv antibody in the PBMC:OV-90 co-culture.

FIG. 28A and FIG. 28B are graphs showing the production of IL-2 (FIG. 28A) and IL-6 levels (FIG. 28B) in by human PBMCs co-cultured with OV-90 cells. FIG. 28A shows antibody induced IL-2 levels for the IMC-16-13 scFv-Fab IgG antibody, IMC-16-15 scFv-Fab IgG antibody, IMC-16-3 scFv-Fab IgG antibody, IMC-2-7 tandem scFv antibody, IMC-2-13 tandem scFv antibody, IMC-20-13 IgG-scFv antibody, IMC-21-1 IgG-(scFv)2 antibody, IMC-21-6 IgG-(scFv)2 antibody, and the IMC-2-3 tandem scFv antibody in the PBMC:OV-90 co-culture. FIG. 28B shows antibody induced IL-6 levels for the IMC-16-13 scFv-Fab IgG antibody, IMC-16-15 scFv-Fab IgG antibody, IMC-16-3 scFv-Fab IgG antibody, IMC-2-7 tandem scFv antibody, IMC-2-13 tandem scFv antibody, IMC-20-13 IgG-scFv antibody, IMC-21-1 IgG-(scFv)2 antibody, IMC-21-6 IgG-(scFv)2 antibody, and the IMC-2-3 tandem scFv antibody in the PBMC:OV-90 co-culture.

FIG. 29A, FIG. 29B, FIG. 29C, and FIG. 29D are graphs showing binding affinity of the IMC-16-3 scFv-Fab IgG antibody (FIG. 29A), IMC-16-15 scFv-Fab IgG antibody (FIG. 29B), IMC-21-1 IgG-(scFv)2 antibody (FIG. 29C), and IMC-2-7 tandem scFv antibody (FIG. 29D) to hsCD368-Biotin.

FIG. 30A and FIG. 30B are graphs of cellular toxicity data from human T-cell dependent cellular cytotoxicity experiments. FIG. 30A shows cytotoxicity data of the IMC-16-3 scFv-Fab IgG antibody, IMC-21-1 IgG-(scFv)₂ antibody, IMC-16-15 scFv-Fab IgG antibody, IMC-2-7 tandem scFv antibody, and a negative control (CKV063 LALA) versus K562 CLDN6 expressing cells. FIG. 30B shows cytotoxicity data of the IMC-16-3 scFv-Fab IgG antibody, IMC-21-1 IgG-(scFv)₂ antibody, IMC-16-15 scFv-Fab IgG antibody, IMC-2-7 tandem scFv antibody, and a negative control (CKV063 LALA) versus OV-90 cells.

The experiments in FIG. 31A and FIG. 31B show cytokine analysis data of the IMC-16-3 scFv-Fab IgG antibody (FIG. 31A), and the IMC-2-7 tandem scFv antibody (FIG. 31B) by human T cells co-cultured with K562 CLDN6 expressing cells. FIG. 31A shows the expression level of TNF-α by human T cells co-cultured with K562 CLDN6 expressing cells, and FIG. 31B shows the expression level of IFN-γ by human T cells co-cultured with K562 CLDN6 expressing cells.

FIG. 32 is a non-limiting image showing the critical residues for monoclonal antibody binding that were identified using a CLDN6 alanine-scanning library expressed in HEK-293 T cells. CLDN6 specificity was determined by recognition of the Q156 γ carbon (FIG. 4). In CLDN9, the native L156 residue leads to steric inhibition of monoclonal antibody binding.

FIG. 33A and FIG. 33B are graphs showing the binding of the IMC-16-3 scFv-Fab IgG antibody is highly specific for CLDN6. FIG. 33A shows binding data of the IMC-16-3 scFv-Fab IgG antibody, IMC-16-15 scFv-Fab IgG antibody, IMC-21-1 IgG-(scFv)₂ antibody, and IMC-2-7 tandem scFv antibody to HEK293-F cells transiently expressing CLDN6. FIG. 33B shows binding data of the IMC-16-3 scFv-Fab IgG antibody, IMC-16-15 scFv-Fab IgG antibody, IMC-21-1 IgG-(scFv)₂ antibody, and IMC-2-7 tandem scFv antibody to HEK293-F cells transiently expressing CLDN9.

FIG. 34 is a graph showing the specific cytotoxicity mediated by the IMC-16-3 scFv-Fab IgG antibody, which is strongly preferential for CLDN6.

FIG. 35A and FIG. 35B are graphs illustrating the activation of CD4 (FIG. 35B) and CD8 (FIG. 35A) T cells by the bispecific antibody IMC-16-3 in the presence of CLDN-6 expressing tumor cells as indicated by expression of the activation markers CD69 and CD25 by human cytotoxic T cells (Tc) and human helper cells (Th).

FIG. 36 illustrates the results of an OV-90 xenograft preclinical animal study with treatment with physiologically relevant doses of IMC-16-3.

FIG. 37A and FIG. 37B illustrate the selectivity of a reference antibody (RefMAB #1, FIG. 37A) and IMC-16-3 (FIG. 37B) for various claudin proteins.

FIG. 38A and FIG. 38B illustrate the result of an internalization assessment of IMC-16-3 and RefMAB #2. FIG. 38A illustrates the percent internalization of the antibodies in a K562 cell line with high expression of CLDN6. FIG. 38B illustrates the percent internalization of the antibodies in OV-90 cells with moderate expression of CLDN6.

FIG. 39A illustrates the results of a OVCAR3 Xenograft tumor study in mice treated with various concentrations of IMC-16-3.

FIG. 39B illustrates the weights of the mice throughout the duration of the study of FIG. 39A.

FIG. 40 illustrates the plasma levels of IMC-16-3 in the plasma of non-human primates dosed with various concentrations of the antibody.

FIG. 41A, FIG. 41B, and FIG. 41C illustrate the comparative selectivity of RefMAB #2 (FIG. 41A), RefMAB #3 (FIG. 41B), and IMC-16-3 (FIG. 41C) for various claudin proteins.

FIG. 42A and FIG. 42B illustrate the results of a comparative T-cell dependent cytotoxicity assay comparing IMC-16-3 and RefMAB #3. FIG. 42A illustrates the results when using a 12.5:1 E:T ratio. FIG. 42B illustrates the results when using a 2.5:1 E:T ratio.

FIG. 43A, FIG. 43B, FIG. 43C, and FIG. 43D illustrate the results of a comparative PBMC cytokine production assay comparing IMC-16-3 and RefMAB #3. FIG. 43A illustrates IL-6 production. FIG. 43B illustrates IL-2 production. FIG. 43C illustrates TNF-α production. FIG. 43D illustrates IL-8 production.

FIG. 44A, FIG. 44B, and FIG. 44C illustrate the results of a comparative K562-CLDN6 killing assay comparing IMC-16-3 and RefMAB #2 in cell lines with varying CLDN6 expression levels and in the presence or absence of human PBMCs. FIG. 44A illustrates the results versus cells expressing high levels of CLDN6. FIG. 44B illustrates the results versus cells expressing moderate levels of CLDN6. FIG. 44C illustrates the results versus cells expressing no CLDN6 (but which have a high rate of nonspecific pinocytosis).

FIG. 45 illustrates pharmacokinetic results of IMC-16-3 in non-human primates dosed with 0.1 mg/kg or 1 mg/kg IMC-16-3.

### DETAILED DESCRIPTION

The present disclosure is based, in part, on the surprising discovery of an antibody specific for claudin 6 and CD3. The present disclosure describes the isolation and characterization of antibodies, antibody fragments, and antibody variants specific for claudin 6 and CD3. In some embodiments, the antibody specific for claudin 6 and CD3 is a tandem single-chain variable fragment (scFv). In some embodiments, the antibody specific for claudin 6 and CD3 is a scFv-Fab Fc antibody specific for claudin 6 and CD3. In some embodiments, the antibody specific for claudin 6 and CD3 is an IgG-(scFV)₂ antibody specific for claudin 6 and CD3. In some embodiments, the antibody specific for claudin 6 and CD3 is a scFv-Fab Fc specific for claudin 6 and CD3. In some embodiments, the antibody specific for claudin 6 and CD3 binds claudin 6 and CD3 contemporaneously.

For example, in some embodiments, the antibody specific for claudin 6 and CD3 is a tandem scFv selected from an amino acid sequence of SEQ ID NO: 82, SEQ ID NO: 83, SEQ ID NO: 84, SEQ ID NO: 85, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 109, SEQ ID NO: 110, or 115, or an amino acid sequence having at least about 90%, or about 93%, or about 95%, or about 97%, or about 98%, or about 99% identity thereto.

In some embodiments, the antibody specific for claudin 6 and CD3 is a scFv-Fab Fc antibody comprising a first heavy chain sequence, a second heavy chain sequence, and a light chain sequence. For example, the antibody specific for claudin 6 and CD3 is selected from a first heavy chain having an amino acid sequence of SEQ ID NO: 79, SEQ ID NO: 114, or SEQ ID NO: 88, a second heavy chain having an amino acid sequence of SEQ ID NO: 80, SEQ ID NO: 89, SEQ ID NO: 90, SEQ ID NO: 91, SEQ ID NO: 92, SEQ ID NO: 111, or SEQ ID NO: 116, and a light chain having an amino acid sequence of SEQ ID NO: 67, SEQ ID NO: 65, or SEQ ID NO: 66. In some embodiments, the first heavy chain comprises an amino acid sequence of SEQ ID NO: 79, SEQ ID NO: 114, or SEQ ID NO: 88, or an amino acid sequence having at least about 90%, or about 93%, or about 95%, or about 97%, or about 98%, or about 99% identity thereto. In some embodiments, the second heavy comprises an amino acid sequence of SEQ ID NO: 80, SEQ ID NO: 89, SEQ ID NO: 90, SEQ ID NO: 91, SEQ ID NO: 92, SEQ ID NO: 111, or SEQ ID NO: 116, or an amino acid sequence having at least about 90%, or about 93%, or about 95%, or about 97%, or about 98%, or about 99% identity thereto. In some embodiments, the light chain comprises an amino acid sequence of SEQ ID NO: 67, SEQ ID NO: 65, or SEQ ID NO: 66, or an amino acid sequence having at least about 90%, or about 93%, or about 95%, or about 97%, or about 98%, or about 99% identity thereto.

In some embodiments, the antibody specific for claudin 6 and CD3 is an IgG-(scFV)₂ antibody comprising a heavy chain selected from an amino acid sequence of SEQ ID NO: 86, SEQ ID NO: 87, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 112, or SEQ ID NO: 113, and a light chain selected from an amino acid sequence of SEQ ID NO: 67, SEQ ID NO: 65, or SEQ ID NO: 66. In some embodiments, the heavy chain comprises an amino acid sequence of SEQ ID NO: 86, SEQ ID NO: 87, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 112, or SEQ ID NO: 113, or an amino acid sequence having at least about 90%, or about 93%, or about 95%, or about 97%, or about 98%, or about 99% identity thereto. In some embodiments, the light chain comprises an amino acid sequence of SEQ ID NO: 67, SEQ ID NO: 65, or SEQ ID NO: 66, or an amino acid sequence having at least about 90%, or about 93%, or about 95%, or about 97%, or about 98%, or about 99% identity thereto.

In some embodiments, the antibody specific for claudin 6 and CD3 is a scFv-Fab Fc antibody comprising a first heavy chain selected from an amino acid sequence of SEQ ID NO: 79, SEQ ID NO: 114, or SEQ ID NO: 88, a second heavy chain selected from an amino acid sequence of SEQ ID NO: 81, and a light chain selected from an amino acid sequence of SEQ ID NO: 67, SEQ ID NO: 65, or SEQ ID NO: 66. In some embodiments, the antibody comprises a first heavy chain amino acid sequence of SEQ ID NO: 79, SEQ ID NO: 114, or SEQ ID NO: 88, or an amino acid sequence having at least about 90%, or about 93%, or about 95%, or about 97%, or about 98%, or about 99% identity thereto. In some embodiments, the antibody comprises a second heavy chain amino acid sequence of SEQ ID NO: 81, or an amino acid sequence having at least about 90%, or about 93%, or about 95%, or about 97%, or about 98%, or about 99% identity thereto. In some embodiments, the antibody comprises a light chain amino acid sequence of SEQ ID NO: 67, SEQ ID NO: 65, or SEQ ID NO: 66, or an amino acid sequence having at least about 90%, or about 93%, or about 95%, or about 97%, or about 98%, or about 99% identity thereto.

In some embodiments, the antibody specific for claudin 6 and CD3 is a scFv-Fab Fc antibody comprising a first heavy chain selected from an amino acid sequence of SEQ ID NO: 79, SEQ ID NO: 114, or SEQ ID NO: 88, a second heavy chain selected from an amino acid sequence of SEQ ID NO: 117, SEQ ID NO: 119, SEQ ID NO: 118, or SEQ ID NO: 120, and a light chain selected from an amino acid sequence of SEQ ID NO: 67, SEQ ID NO: 65, or SEQ ID NO: 66. In some embodiments, the antibody comprises a first heavy chain amino acid sequence of SEQ ID NO: 79, SEQ ID NO: 114, or SEQ ID NO: 88, or an amino acid sequence having at least about 90%, or about 93%, or about 95%, or about 97%, or about 98%, or about 99% identity thereto. In some embodiments, the antibody comprises a second heavy chain amino acid sequence of SEQ ID NO: 117, SEQ ID NO: 119, SEQ ID NO: 118, or SEQ ID NO: 120, or an amino acid sequence having at least about 90%, or about 93%, or about 95%, or about 97%, or about 98%, or about 99% identity thereto. In some embodiments, the antibody comprises a light chain amino acid sequence of SEQ ID NO: 67, SEQ ID NO: 65, or SEQ ID NO: 66, or an amino acid sequence having at least about 90%, or about 93%, or about 95%, or about 97%, or about 98%, or about 99% identity thereto.

In some embodiments, the antibody specific for claudin 6 and CD3 is a scFv-Fab Fc antibody comprising a first heavy chain selected from an amino acid sequence of SEQ ID NO: 79, SEQ ID NO: 114, or SEQ ID NO: 88, a second heavy chain selected from an amino acid sequence of SEQ ID NO: 121, SEQ ID NO: 123, SEQ ID NO: 125, SEQ ID NO: 127, SEQ ID NO: 129, a first light selected from an chain amino acid sequence of SEQ ID NO: 67, SEQ ID NO: 65, or SEQ ID NO: 66, and a second light chain selected from an amino acid sequence of SEQ ID NO: 122, SEQ ID NO: 124, SEQ ID NO: 126, SEQ ID NO: 128, or SEQ ID NO: 130. In some embodiments, the antibody comprises a first heavy chain selected from an amino acid sequence of SEQ ID NO: 79, SEQ ID NO: 114, or SEQ ID NO: 88, or an amino acid sequence having at least about 90%, or about 93%, or about 95%, or about 97%, or about 98%, or about 99% identity thereto. In some embodiments, the antibody comprises a second heavy chain selected from an amino acid sequence of SEQ ID NO: 121, SEQ ID NO: 123, SEQ ID NO: 125, SEQ ID NO: 127, SEQ ID NO: 129, or an amino acid sequence having at least about 90%, or about 93%, or about 95%, or about 97%, or about 98%, or about 99% identity thereto. In some embodiments, the antibody comprises a first light selected from an chain amino acid sequence of SEQ ID NO: 67, SEQ ID NO: 65, or SEQ ID NO: 66, or an amino acid sequence having at least about 90%, or about 93%, or about 95%, or about 97%, or about 98%, or about 99% identity thereto. In some embodiments, the antibody comprises a second light chain selected from an amino acid sequence of SEQ ID NO: 122, SEQ ID NO: 124, SEQ ID NO: 126, SEQ ID NO: 128, or SEQ ID NO: 130, or an amino acid sequence having at least about 90%, or about 93%, or about 95%, or about 97%, or about 98%, or about 99% identity thereto.

In various embodiments, the antibody (e.g., a tandem scFv antibody, scFv-Fab Fc antibody, IgG-(scFV)₂ antibody, and a scFv-Fab Fc antibody), or fragment thereof, or variant thereof, may comprise an amino acid sequence having one or more amino acid mutations (e.g., substitutions or deletions) relative to any of the sequences disclosed herein. In some embodiments, the one or more amino acid mutations may be independently selected from substitutions, insertions, deletions, and truncations. In embodiments, the antibody (e.g. a tandem scFv antibody, scFv-Fab Fc antibody, IgG-(scFV)₂ antibody, and a scFv-Fab Fc antibody), or fragment thereof, or variant thereof, comprises a sequence that has about 1, 2, 3, 4, 5, 6, 7, 8, 9, or 10 or more amino acid mutations with respect to any one of the amino acid sequences disclosed herein.

In various embodiments, the antibody or antibody format (e.g. a tandem scFv antibody, scFv-Fab Fc antibody, IgG-(scFV)₂ antibody, and a scFv-Fab Fc antibody), or fragment thereof, or variant thereof, may comprise an amino acid sequence having at least about 50%, about 60%, about 70%, about 80%, about 90%, about 95%, about 96%, about 97%, about 98%, about 99% sequence homology to the amino acid sequences disclosed herein.

In various embodiments, disclosed herein are variants or fragments comprising any of the sequences described herein, for instance, a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with any of the sequences disclosed herein.

In some embodiments, variants are those that have conservative amino acid substitutions made at one or more predicted non-essential amino acid residues. For example, a “conservative amino acid substitution” is one in which the amino acid residue is replaced with an amino acid residue having a similar side chain.

In embodiments, the amino acid mutations are amino acid substitutions, and may include conservative and/or non-conservative substitutions. Throughout the present disclosure, the term “mutation” and “substitution” may be used interchangeably when the context allows. For example, a substitution of threonine at position 366 of a protein to a serine may be referred to as a T366S substitution or a T366S mutation. The skilled artisan would readily understand that “T366S” connotes a substitution of threonine for serine at position 366, and thus the skilled artisan would readily understand that “T366S mutation” refers to a substitution.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

As disclosed herein, the term “antibody” refers to a broad sense and includes immunoglobulin or antibody molecules including polyclonal antibodies, monoclonal antibodies including murine, human, humanized and chimeric monoclonal antibodies and antibody fragments, such as ScFv (PLOS Biology|DOI:10.1371/journal.pbio.1002344 Jan. 6, 2016, which is hereby incorporated by reference in its entirety).

In general, antibodies are proteins or polypeptides that exhibit binding specificity to a specific antigen. Intact antibodies are heterotetrameric glycoproteins, composed of two identical light chains and two identical heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa and lambda, based on the amino acid sequences of their constant domains. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4.

As disclosed herein, the term “antibody fragment” refers to an intact antibody, generally the antigen binding or variable region of the intact antibody. Examples of antibody fragments include Fab, Fab′, F(ab′)2 and Fv fragments, diabodies, single chain antibody molecules and multispecific antibodies formed from at least two intact antibodies or fragments thereof.

As disclosed herein, the term “antigen” refers to any molecule that has the ability to generate antibodies either directly or indirectly.

As disclosed herein, the term “specific binding” or “immunospecific binding” or “binds immunospecifically” refers to antibody binding to a predetermined antigen (e.g., Claudin 6) or epitope present on the antigen. In some embodiments, the antibody binds with a dissociation constant (KD) of about 10⁻¹⁰ M or less, of about 10⁻⁹ M or less, of about 10⁻⁸ M or less, of about 10⁻⁷ M or less, of about 10⁻⁶ M or less, of about 10⁻⁵ M or less, and binds to the predetermined antigen with a KD that is at least two-fold less than its KD for binding to a non-specific antigen (e.g., BSA, casein, or another non-specific polypeptide) other than the predetermined antigen. The phrases “an antibody recognizing Claudin 6” and “an antibody specific for Claudin 6” are used interchangeably herein with the term “an antibody which binds immunospecifically to Claudin 6.” Reference in the present disclosure may be made to Claudin 6. In some embodiments, the antibody is specific for Claudin 6 and does not specifically bind to claudin 3, claudin 4, and/or claudin 9.

“CDRs” are referred to as the complementarity determining region amino acid sequences of an antibody which are the hypervariable regions of immunoglobulin heavy and light chains. See, e.g., Kabat et al., Sequences of Proteins of Immunological Interest, 4th ed., U.S. Department of Health and Human Services, National Institutes of Health (1987). There are three heavy chain and three light chain CDRs or CDR regions in the variable portion of an immunoglobulin. Thus, “CDRs” as used herein refers to all three heavy chain CDRs, or all three light chain CDRs or both all heavy and all light chain CDRs, if appropriate.

Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity, as the CDRs provide the majority of contact residues for the binding of the antibody to the antigen or epitope. CDRs of interest can be derived from donor antibody variable heavy and light chain sequences, and include analogs of the naturally occurring CDRs, which analogs also share or retain the same antigen binding specificity and/or neutralizing ability as the donor antibody from which they were derived. In some embodiments, the antibody is a chimeric antibody. In some embodiments, the antibody is a humanized antibody.

CDRs are based on sequence variability (Wu and Kabat, J. Exp. Med. 132:211-250, 1970). There are six CDRs--three in the variable heavy chain, or VH, and are typically designated H-CDR1, H-CDR2, and H-CDR3, and three CDRs in the variable light chain, or VL, and are typically designated L-CDR1, L-CDR2, and L-CDR3 (Kabat et al., Sequences of Proteins of Immunological Interest, 5th Ed. Public Health Service, National Institutes of Health, Bethesda, Md., 1991). “Hypervariable region”, “HVR”, or “HV” refer to the regions of an antibody variable domain which are variable in structure as defined by Chothia and Lesk (Chothia and Lesk, Mol. Biol. 196:901-917, 1987). There are six HVRs, three in VH (H1, H2, H3) and three in VL (L1, L2, L3). Chothia and Lesk refer to structurally conserved HVs as “canonical structures.” Another method of describing the regions that form the antigen-binding site has been proposed by Lefranc (Lefranc et al., Developmental & Comparative Immunology 27:55-77, 2003) based on the comparison of V domains from immunoglobulins and T-cell receptors (Lefranc et al., Developmental & Comparative Immunology 27:55-77, 2003). The antigen-binding site can also be delineated based on “Specificity Determining Residue Usage (SDRU)”, according to Almagro (Almagro, Mol. Recognit. 17:132-43, 2004), where SDRU refers to amino acid residues of an immunoglobulin that are directly involved in antigen contact.

An “isolated antibody,” as used herein, refers to an antibody that is substantially free of other antibodies having different antigenic specificities (e.g., an isolated antibody that specifically binds Claudin 6 is substantially free of antibodies that specifically bind antigens other than Claudin 6). Moreover, an isolated antibody may be substantially free of other cellular material and/or chemicals. An isolated antibody can also be sterile or pyrogen free or formulated as injectable pharmaceutical as described herein.

In some embodiments, the source for the DNA encoding a non-human antibody include cell lines which produce antibody, such as hybrid cell lines commonly known as hybridomas.

### Claudin 6/CD3 Binders

In some embodiments, the antibody specific for claudin 6 and CD3 (e.g. a tandem scFv antibody, scFv-Fab Fc antibody, IgG-(scFV)₂ antibody, and a scFv-Fab Fc antibody), or fragment thereof, or variant thereof, is selected from one of the following amino acid sequences. Bold font refers to a CDR based on Kabat designations, and the underline formatting refers to a linker.

>SEQ ID NO: 82
(SEQ ID NO: 82)
SYELTQPPSVSVSPGQTARITCSAGSGLYGWYQQKPGQAPVLV
IYGTNKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDAWGQGTLVTVSSGGGGSDI
KLQQSGAELARPGASVKMSCKTSGYTFTRYTMHWVKQRPGQ
GLEWIGYINPSRGYTNYNQKFKDKATLTTDKSSSTAYMQLSS
LTSEDSAVYYCARYYDDHYCLDYWGQGTTLTVSSVEGGSGGS
GGSGGSGGVDDIQLTQSPAIMSASPGEKVTMTCRASSSVSYMN
WYQQKSGTSPKRWIYDTSKVASGVPYRFSGSGSGTSYSLTISS
MEAEDAATYYCQQWSSNPLTFGAGTKLELK
>SEQ ID NO: 83
(SEQ ID NO: 83)
SYELTQPPSVSVSPGQTARITCSGGSGSYGWYQQKPGQAPVLV
IYGTYKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDVWGQGTLVTVSSGGGGSDI
KLQQSGAELARPGASVKMSCKTSGYTFTRYTMHWVKQRPGQ
GLEWIGYINPSRGYTNYNQKFKDKATLTTDKSSSTAYMQLSS
LTSEDSAVYYCARYYDDHYCLDYWGQGTTLTVSSVEGGSGGS
GGSGGSGGVDDIQLTQSPAIMSASPGEKVTMTCRASSSVSYMN
WYQQKSGTSPKRWIYDTSKVASGVPYRFSGSGSGTSYSLTISS
MEAEDAATYYCQQWSSNPLTFGAGTKLELK
>SEQ ID NO: 84
(SEQ ID NO: 84)
SYELTQPPSVSVSPGQTARITCSAGSGLYGWYQQKPGQAPVLV
IYGTNKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDAWGQGTLVTVSSGGGGSEV
QLVESGGGPVQAGGSLRLSCAASGRTYRGYSMGWFRQAPGK
EREFVAAIVWSGGNTYYEDSVKGRFTISRDNAKNTMYLQMTS
LKPEDSATYYCAAKIRPYIFKIAGQYDYWGQGTQVTVSS
>SEQ ID NO: 85
(SEQ ID NO: 85)
SYELTQPPSVSVSPGQTARITCSGGSGSYGWYQQKPGQAPVLV
IYGTYKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDVWGQGTLVTVSSGGGGSEV
QLVESGGGPVQAGGSLRLSCAASGRTYRGYSMGWFRQAPGK
EREFVAAIVWSGGNTYYEDSVKGRFTISRDNAKNTMYLQMTS
LKPEDSATYYCAAKIRPYIFKIAGQYDYWGQGTQVTVSS
>SEQ ID NO: 93
(SEQ ID NO: 93)
SYELTQPPSVSVSPGQTARITCSAGSGLYGWYQQKPGQAPVLV
IYGTNKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDAWGQGTLVTVSSGGGGSEV
QLVESGGGLVQPGGSLKLSCAASGFTFNKYAMNWVRQAPGK
GLEWVARIRSKYNNYATYYADSVKDRFTISRDDSKNTAYLQM
NNLKTEDTAVYYCVRHGNFGNSYISYWAYWGQGTLVTVSSG
GGGSGGGGSGGGGSQTVVTQEPSLTVSPGGTVTLTCGSSTGAV
TSGNYPNWVQQKPGQAPRGLIGGTKFLAPGTPARFSGSLLGG
KAALTLSGVQPEDEAEYYCVLWYSNRWVFGGGTKLTVL
>SEQ ID NO: 94
(SEQ ID NO: 94)
SYELTQPPSVSVSPGQTARITCSGGSGSYGWYQQKPGQAPVLV
IYGTYKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDVWGQGTLVTVSSGGGGSEV
QLVESGGGLVQPGGSLKLSCAASGFTFNKYAMNWVRQAPGK
GLEWVARIRSKYNNYATYYADSVKDRFTISRDDSKNTAYLQM
NNLKTEDTAVYYCVRHGNFGNSYISYWAYWGQGTLVTVSSG
GGGSGGGGSGGGGSQTVVTQEPSLTVSPGGTVTLTCGSSTGAV
TSGNYPNWVQQKPGQAPRGLIGGTKFLAPGTPARFSGSLLGG
KAALTLSGVQPEDEAEYYCVLWYSNRWVFGGGTKLTVL
>SEQ ID NO: 95
(SEQ ID NO: 95)
SYELTQPPSVSVSPGQTARITCSAGSGLYGWYQQKPGQAPVLV
IYGTNKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDAWGQGTLVTVSSGGGGSEV
QLVESGGGLVQPGGSLRLSCAASGFTFSTYAMNWVRQAPGKG
LEWVGRIRSKYNNYATYYADSVKGRFTISRDDSKNTLYLQMN
SLRAEDTAVYYCVRHGNFGDSYVSWFAYWGQGTLVTVSSGK
PGSGKPGSGKPGSGKPGSQAVVTQEPSLTVSPGGTVTLTCGSST
GAVTTSNYANWVQQKPGKSPRGLIGGTNKRAPGVPARFSGSL
LGGKAALTISGAQPEDEADYYCALWYSNHWVFGGGTKLTVL
>SEQ ID NO: 115
(SEQ ID NO: 115)
SYELTQPPSVSVSPGQTARITCSAGSGLYGWYQQKPGQAPVLVIYGT
NKRPSGIPERFSGSSSGTTVTLTISGVQAEDEADYYCGSADSSTNAGIF
GGGTKLTVLGGGGSGGGGSGGGGSEVQLLESGGGLVQPGGSLRLSCA
ASGFTFSSYAMNWVRQAPGKGLEWVAGISSSGRYTGYADSVKGRFT
ISRDNSKNTLYLQMNSLRAEDTAVYYCAKSMGSGVSWSGYVATSID
VWGQGTLVTVSSGGGGSDIKLQQSGAELARPGASVKMSCKTSGYTFT
RYTMHWVKQRPGQGLEWIGYINPSRGYTNYNQKFKDKATLTTDKS
SSTAYMQLSSLTSEDSAVYYCARYYDDHYCLDYWGQGTTLTVSSVE
GGSGGSGGSGGSGGVDDIQLTQSPAIMSASPGEKVTMTCRASSSVSY
MNWYQQKSGTSPKRWIYDTSKVASGVPYRFSGSGSGTSYSLTISSME
AEDAATYYCQQWSSNPLTFGAGTKLELK
>SEQ ID NO: 96
(SEQ ID NO: 96)
SYELTQPPSVSVSPGQTARITCSGGSGSYGWYQQKPGQAPVLV
IYGTYKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDVWGQGTLVTVSSGGGGSEV
QLVESGGGLVQPGGSLRLSCAASGFTFSTYAMNWVRQAPGKG
LEWVGRIRSKYNNYATYYADSVKGRFTISRDDSKNTLYLQMN
SLRAEDTAVYYCVRHGNFGDSYVSWFAYWGQGTLVTVSSGK
PGSGKPGSGKPGSGKPGSQAVVTQEPSLTVSPGGTVTLTCGSST
GAVTTSNYANWVQQKPGKSPRGLIGGTNKRAPGVPARFSGSL
LGGKAALTISGAQPEDEADYYCALWYSNHWVFGGGTKLTVL
>SEQ ID NO: 97
(SEQ ID NO: 97)
SYELTQPPSVSVSPGQTARITCSAGSGLYGWYQQKPGQAPVLV
IYGTNKRPSGIPERFSGSSSGTTVTLTISGVQAEDEADYYCGSA
DSSTNAGIFGGGTKLTVLGGGGSGGGGGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDAWGQGTLVTVSSGGGGSEV
QLVESGGGLVQPGGSLRLSCAASGFTFNTYAMNWVRQAPGKG
LEWVARIRSKYNNYATYYADSVKSRTTISDDSKNTLYLQMNS
LRAEDTAVYYCVRHGNFGNSYVSWFAYWGQTTVTVSSGGGG
SGGGGSGGGGSGGGGSEIVVTQSPATLSVSPGERATLSCRSSTG
AVTTSNYANWVQEPGQAFRGLIGGANKRAPGVPARFSGSLSG
DEATLTISSLQSEDFAVYYCALWYSNLWVFGQGTKLEIK
>SEQ ID NO: 98
(SEQ ID NO: 98)
SYELTQPPSVSVSPGQTARITCSGGSGSYGWYQQKPGQAPVLV
IYGTYKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDVWGQGTLVTVSSGGGGSEV
QLVESGGGLVQPGGSLRLSCAASGFTFNTYAMNWVRQAPGKG
LEWVARIRSKYNNYATYYADSVKSRTTISDDSKNTLYLQMNS
LRAEDTAVYYCVRHGNFGNSYVSWFAYWGQTTVTVSSGGGG
SGGGGSGGGGSGGGGSEIVVTQSPATLSVSPGERATLSCRSSTG
AVTTSNYANWVQEPGQAFRGLIGGANKRAPGVPARFSGSLSG
DEATLTISSLQSEDFAVYYCALWYSNLWVFGQGTKLEIK
>SEQ ID NO: 99
(SEQ ID NO: 99)
SYELTQPPSVSVSPGQTARITCSAGSGLYGWYQQKPGQAPVLV
IYGTNKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDAWGQGTLVTVSSGGGGSQV
QLVQSGGGVVQPGRSLRLSCKASGYTFTRYTMHWVRQAPGK
GLEWIGYINPSRGYTNYNQKVKDRFTISTDKSKSTAFLQMDSL
RPEDTAVYYCARYYDDHYCLDYWGQGTTLTVSSGGGGSGGG
GSGGGGSDIQMTQSPSSLSASVGDRVTITCSASSSVSYMNWYQ
QTPGKAPKRWIYDTSKLASGVPSRFSGSGSGTDYTFTISSLQPE
DIATYYCQQWSSNPFTFGQGTKLQITR
>SEQ ID NO: 100
(SEQ ID NO: 100)
SYELTQPPSVSVSPGQTARITCSGGSGSYGWYQQKPGQAPVLV
IYGTYKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDVWGQGTLVTVSSGGGGSQV
QLVQSGGGVVQPGRSLRLSCKASGYTFTRYTMHWVRQAPGK
GLEWIGYINPSRGYTNYNQKVKDRFTISTDKSKSTAFLQMDSL
RPEDTAVYYCARYYDDHYCLDYWGQGTTLTVSSGGGGSGGG
GSGGGGSDIQMTQSPSSLSASVGDRVTITCSASSSVSYMNWYQ
QTPGKAPKRWIYDTSKLASGVPSRFSGSGSGTDYTFTISSLQPE
DIATYYCQQWSSNPFTFGQGTKLQITR
>SEQ ID NO: 109
(SEQ ID NO: 109)
SYELTQPPSVSVSPGQTARITCSAGSGLYGWYQQKPGQAPVLV
IYGTNKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDAWGQGTLVTVSSGGGGSEV
QLLESGGGLVQPGGSLRLSCAASGFSFTGYTMNWVRQAPGKG
LEWMGLINPYKGVSTYNQKVKGRFTISRDNSKNTLYLQMNSL
RAEDTAVYYCARSGYYGDSDWYFDVWGQGTLVTVSSGGGGS
GGGGSGGGGSDIQMTQSPSSLSASVGDRVTITCRASQDIRNYL
NWYQQTPGKAPKRWIYYTSRLHSGVPSRFSGSGSGTDYTFTYS
SLQPEDIATYYCQQGNTLPWTFGQGTKLEIK
>SEQ ID NO: 110
(SEQ ID NO: 110)
SYELTQPPSVSVSPGQTARITCSGGSGSYGWYQQKPGQAPVLV
IYGTYKRPSGIPERFSGSSSGTTVTLTISGVQAEDEADYYCGSA
DSSTNAGIFGGGTKLTVLGGGGSGGGGSGGGGSEVQLLESGG
GLVQPGGSLRLSCAASGFTFSSYAMNWVRQAPGKGLEWVAGI
SSSGRYTGYADSVKGRFTISRDNSKNTLYLQMNSLRAEDTAV
YYCAKSVGSGVSWSGYVATSLDVWGQGTLVTVSSGGGGSEV
QLLESGGGLVQPGGSLRLSCAASGFSFTGYTMNWVRQAPGKG
LEWMGLINPYKGVSTYNQKVKGRFTISRDNSKNTLYLQMNSL
RAEDTAVYYCARSGYYGDSDWYFDVWGQGTLVTVSSGGGGS
GGGGSGGGGSDIQMTQSPSSLSASVGDRVTITCRASQDIRNYL
NWYQQTPGKAPKRWIYYTSRLHSGVPSRFSGSGSGTDYTFTYS
SLQPEDIATYYCQQGNTLPWTFGQGTKLEIK
>SEQ ID NO: 79
(SEQ ID NO: 79)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDAWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSREEMTKNQVSLWCLVKGFYPS
DIAVEWESNGQPENNYKTTPPVLDSDGSFFLYSKLTVDKSRWQ
QGNVFSCSVMHEALHNHYTQKSLSLSPGK
>SEQ ID NO: 88
(SEQ ID NO: 88)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDVWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSREEMTKNQVSLWCLVKGFYPS
DIAVEWESNGQPENNYKTTPPVLDSDGSFFLYSKLTVDKSRWQ
QGNVFSCSVMHEALHNHYTQKSLSLSPGK
>SEQ ID NO: 114
(SEQ ID NO: 114)
EVQLLESGGGLVQPGGSLRLSCAASGFTFSSYAMNWVRQAPGKGLE
WVAGISSSGRYTGYADSVKGRFTISRDNSKNTLYLQMNSLRAEDTAV
YYCAKSMGSGVSWSGYVATSIDVWGQGTLVTVSSASTKGPSVFPLA
PSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHT
CPPCPAPEAAGGPSVFLFPPKPKDTLMISRTPEVTCVVVDVSHEDPEVK
FNWYVDGVEVHNAKTKPREEQYNSTYRVVSVLTVLHQDWLNGKEY
KCKVSNKALPAPIEKTISKAKGQPREPQVYTLPPSREEMTKNQVSLWC
LVKGFYPSDIAVEWESNGQPENNYKTTPPVLDSDGSFFLYSKLTVDKS
RWQQGNVFSCSVMHEALHNHYTQKSLSLSPGK
>SEQ ID NO: 80
(SEQ ID NO: 80)
EVQLVESGGGPVQAGGSLRLSCAASGRTYRGYSMGWFRQAP
GKEREFVAAIVWSGGNTYYEDSVKGRFTISRDNAKNTMYLQ
MTSLKPEDSATYYCAAKIRPYIFKIAGQYDYWGQGTQVTVSS
ASPKSSDKTHTCPPCPAPEAAGGPSVFLFPPKPKDTLMISRTPEV
TCVVVDVSHEDPEVKFNWYVDGVEVHNAKTKPREEQYNSTY
RVVSVLTVLHQDWLNGKEYKCKVSNKALPAPIEKTISKAKGQP
REPQVYTLPPSREEMTKNQVSLSCAVKGFYPSDIAVEWESNGQ
PENNYKTTPPVLDSDGSFFLVSKLTVDKSRWQQGNVFSCSVMH
EALHNHYTQKSLSLSPGK
>SEQ ID NO: 89
(SEQ ID NO: 89)
EVQLVESGGGLVQPGGSLRLSCAASGFTFSTYAMNWVRQAPG
KGLEWVGRIRSKYNNYATYYADSVKGRFTISRDDSKNTLYLQ
MNSLRAEDTAVYYCVRHGNFGDSYVSWFAYWGQGTLVTVSS
GKPGSGKPGSGKPGSGKPGSQAVVTQEPSLTVSPGGTVTLTCG
SSTGAVTTSNYANWVQQKPGKSPRGLIGGTNKRAPGVPARFS
GSLLGGKAALTISGAQPEDEADYYCALWYSNHWVFGGGTKL
TVLASPKSSDKTHTCPPCPAPEAAGGPSVFLFPPKPKDTLMISRT
PEVTCVVVDVSHEDPEVKFNWYVDGVEVHNAKTKPREEQYNS
TYRVVSVLTVLHQDWLNGKEYKCKVSNKALPAPIEKTISKAKG
QPREPQVYTLPPSREEMTKNQVSLSCAVKGFYPSDIAVEWESN
GQPENNYKTTPPVLDSDGSFFLVSKLTVDKSRWQQGNVFSCSV
MHEALHNHYTQKSLSLSPGK
>SEQ ID NO: 90
(SEQ ID NO: 90)
EVQLVESGGGLVQPGGSLRLSCAASGFTFNTYAMNWVRQAPG
KGLEWVARIRSKYNNYATYYADSVKSRTTISDDSKNTLYLQM
NSLRAEDTAVYYCVRHGNFGNSYVSWFAYWGQTTVTVSSGG
GGSGGGGSGGGGSGGGGSEIVVTQSPATLSVSPGERATLSCRSS
TGAVTTSNYANWVQEPGQAFRGLIGGANKRAPGVPARFSGSL
SGDEATLTISSLQSEDFAVYYCALWYSNLWVFGQGTKLEIKAS
PKSSDKTHTCPPCPAPEAAGGPSVFLFPPKPKDTLMISRTPEVTC
VVVDVSHEDPEVKFNWYVDGVEVHNAKTKPREEQYNSTYRV
VSVLTVLHQDWLNGKEYKCKVSNKALPAPIEKTISKAKGQPRE
PQVYTLPPSREEMTKNQVSLSCAVKGFYPSDIAVEWESNGQPE
NNYKTTPPVLDSDGSFFLVSKLTVDKSRWQQGNVFSCSVMHE
ALHNHYTQKSLSLSPGK
>SEQ ID NO: 91
(SEQ ID NO: 91)
EVQLVESGGGLVQPGGSLKLSCAASGFTFNKYAMNWVRQAP
GKGLEWVARIRSKYNNYATYYADSVKDRFTISRDDSKNTAYL
QMNNLKTEDTAVYYCVRHGNFGNSYISYWAYWGQGTLVTV
SSGGGGSGGGGSGGGGSQTVVTQEPSLTVSPGGTVTLTCGSST
GAVTSGNYPNWVQQKPGQAPRGLIGGTKFLAPGTPARFSGSL
LGGKAALTLSGVQPEDEAEYYCVLWYSNRWVFGGGTKLTVL
ASPKSSDKTHTCPPCPAPEAAGGPSVFLFPPKPKDTLMISRTPEV
TCVVVDVSHEDPEVKFNWYVDGVEVHNAKTKPREEQYNSTY
RVVSVLTVLHQDWLNGKEYKCKVSNKALPAPIEKTISKAKGQP
REPQVYTLPPSREEMTKNQVSLSCAVKGFYPSDIAVEWESNGQ
PENNYKTTPPVLDSDGSFFLVSKLTVDKSRWQQGNVFSCSVMH
EALHNHYTQKSLSLSPGK
>SEQ ID NO: 116
(SEQ ID NO: 116)
EVQLVESGGGPVQAGGSLRLSCAASGRTYRGYSMGWFRQAPG
KEREFVAAIVWSGGNTYYEDSVKGRFTISRDNAKNTMYLQMT
SLKPEDSATYYCAAKIRPYIFKIAGQYDYWGQGTQVTVSSGGG
GSGGGGSGGGGSEVQLLESGGGLVQPGGSLRLSCAASGFTFSS
YAMNWVRQAPGKGLEWVAGISSSGRYTGYADSVKGRFTISRD
NSKNTLYLQMNSLRAEDTAVYYCAKSMGSGVSWSGYVATSID
VWGQGTLVTVSSASTKGPSVFPLAPSSKSTSGGTAALGCLVKD
YFPEPVTVSWNSGALTSGVHTFPAVLQSSGLYSLSSVVTVPSSS
LGTQTYICNVNHKPSNTKVDKKVEPKSCDKTHTCPPCPAPEAA
GGPSVFLFPPKPKDTLMISRTPEVTCVVVDVSHEDPEVKFNWY
VDGVEVHNAKTKPREEQYNSTYRVVSVLTVLHQDWLNGKEY
KCKVSNKALPAPIEKTISKAKGQPREPQVYTLPPSREEMTKNQV
SLSCAVKGFYPSDIAVEWESNGQPENNYKTTPPVLDSDGSFFLV
SKLTVDKSRWQQGNVFSCSVMHEALHNHYTQKSLSLSPGK
>SEQ ID NO: 92
(SEQ ID NO: 92)
QVQLVQSGGGVVQPGRSLRLSCKASGYTFTRYTMHWVRQAP
GKGLEWIGYINPSRGYTNYNQKVKDRFTISTDKSKSTAFLQM
DSLRPEDTAVYYCARYYDDHYCLDYWGQGTTLTVSSGGGGS
GGGGSGGGGSDIQMTQSPSSLSASVGDRVTITCSASSSVSYMN
WYQQTPGKAPKRWIYDTSKLASGVPSRFSGSGSGTDYTFTISSL
QPEDIATYYCQQWSSNPFTFGQGTKLQITRASPKSSDKTHTCPP
CPAPEAAGGPSVFLFPPKPKDTLMISRTPEVTCVVVDVSHEDPE
VKFNWYVDGVEVHNAKTKPREEQYNSTYRVVSVLTVLHQDW
LNGKEYKCKVSNKALPAPIEKTISKAKGQPREPQVYTLPPSREE
MTKNQVSLSCAVKGFYPSDIAVEWESNGQPENNYKTTPPVLDS
DGSFFLVSKLTVDKSRWQQGNVFSCSVMHEALHNHYTQKSLS
LSPGK
>SEQ ID NO: 111
(SEQ ID NO: 111)
EVQLLESGGGLVQPGGSLRLSCAASGFSFTGYTMNWVRQAPG
KGLEWMGLINPYKGVSTYNQKVKGRFTISRDNSKNTLYLQM
NSLRAEDTAVYYCARSGYYGDSDWYFDVWGQGTLVTVSSGG
GGSGGGGSGGGGSDIQMTQSPSSLSASVGDRVTITCRASQDIR
NYLNWYQQTPGKAPKRWIYYTSRLHSGVPSRFSGSGSGTDYT
FTYSSLQPEDIATYYCQQGNTLPWTFGQGTKLEIKASPKSSDK
THTCPPCPAPEAAGGPSVFLFPPKPKDTLMISRTPEVTCVVVDV
SHEDPEVKFNWYVDGVEVHNAKTKPREEQYNSTYRVVSVLTV
LHQDWLNGKEYKCKVSNKALPAPIEKTISKAKGQPREPQVYTL
PPSREEMTKNQVSLSCAVKGFYPSDIAVEWESNGQPENNYKTT
PPVLDSDGSFFLVSKLTVDKSRWQQGNVFSCSVMHEALHNHY
TQKSLSLSPGK
>SEQ ID NO: 67
(SEQ ID NO: 67)
SYELTQPPSVSVSPGQTARITCSAGSGLYGWYQQKPGQAPVLV
IYGTNKRPSGIPERFSGSSSGTTVTLTISGVQAEDEADYYCGSA
DSSTNAGIFGGGTKLTVLGQPKAAPSVTLFPPSSEELQANKATL
VCLISDFYPGAVTVAWKADSSPVKAGVETTTPSKQSNNKYAAS
SYLSLTPEQWKSHRSYSCQVTHEGSTVEKTVAPTECS
>SEQ ID NO: 65
(SEQ ID NO: 65)
SYELTQPPSVSVSPGQTARITCSAGSGLYGWYQQKPGQAPVLVIYGT
NKRPSGIPERFSGSSSGTTVTLTISGVQAEDEADYYCGSADSSTNAGIF
GGGTKLTVLGQPKAAPSVTLFPPSSEELQANKATLVCLISDFYPGAVT
VAWKADSSPVKAGVETTTPSKQSNNKYAASSYLSLTPEQWKSHRSYS
CQVTHEGSTVEKTVAPTECS
>SEQ ID NO: 66
(SEQ ID NO: 66)
SYELTQPPSVSVSPGQTARITCSGGSGSYGWYQQKPGQAPVLV
IYGTYKRPSGIPERFSGSSSGTTVTLTISGVQAEDEADYYCGSA
DSSTNAGIFGGGTKLTVLGQPKAAPSVTLFPPSSEELQANKATL
VCLISDFYPGAVTVAWKADSSPVKAGVETTTPSKQSNNKYAAS
SYLSLTPEQWKSHRSYSCQVTHEGSTVEKTVAPTECS
>SEQ ID NO: 86
(SEQ ID NO: 86)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDAWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSEVQLVESGGGPVQAGGSLRLSCAASGRTYRGYSMGWFRQ
APGKEREFVAAIVWSGGNTYYEDSVKGRFTISRDNAKNTMYL
QMTSLKPEDSATYYCAAKIRPYIFKIAGQYDYWGQGTQVTVS
S
>SEQ ID NO: 87
(SEQ ID NO: 87)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDVWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSEVQLVESGGGPVQAGGSLRLSCAASGRTYRGYSMGWFRQ
APGKEREFVAAIVWSGGNTYYEDSVKGRFTISRDNAKNTMYL
QMTSLKPEDSATYYCAAKIRPYIFKIAGQYDYWGQGTQVTVS
S
>SEQ ID NO: 101
(SEQ ID NO: 101)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDAWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSQVQLVQSGGGVVQPGRSLRLSCKASGYTFTRYTMHWVRQ
APGKGLEWIGYINPSRGYTNYNQKVKDRFTISTDKSKSTAFLQ
MDSLRPEDTAVYYCARYYDDHYCLDYWGQGTTLTVSSGGGG
SGGGGSGGGGSDIQMTQSPSSLSASVGDRVTITCSASSSVSYMN
WYQQTPGKAPKRWIYDTSKLASGVPSRFSGSGSGTDYTFTISSL
QPEDIATYYCQQWSSNPFTFGQGTKLQITR
>SEQ ID NO: 102
(SEQ ID NO: 102)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDVWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSQVQLVQSGGGVVQPGRSLRLSCKASGYTFTRYTMHWVRQ
APGKGLEWIGYINPSRGYTNYNQKVKDRFTISTDKSKSTAFLQ
MDSLRPEDTAVYYCARYYDDHYCLDYWGQGTTLTVSSGGGG
SGGGGSGGGGSDIQMTQSPSSLSASVGDRVTITCSASSSVSYMN
WYQQTPGKAPKRWIYDTSKLASGVPSRFSGSGSGTDYTFTISSL
QPEDIATYYCQQWSSNPFTFGQGTKLQITR
>SEQ ID NO: 103
(SEQ ID NO: 103)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDAWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSEVQLVESGGGLVQPGGSLRLSCAASGFTFSTYAMNWVRQA
PGKGLEWVGRIRSKYNNYATYYADSVKGRFTISRDDSKNTLY
LQMNSLRAEDTAVYYCVRHGNFGDSYVSWFAYWGQGTLVT
VSSGKPGSGKPGSGKPGSGKPGSQAVVTQEPSLTVSPGGTVTLT
CGSSTGAVTTSNYANWVQQKPGKSPRGLIGGTNKRAPGVPA
RFSGSLLGGKAALTISGAQPEDEADYYCALWYSNHWVFGGGT
KLTVL
>SEQ ID NO: 104
(SEQ ID NO: 104)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDVWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSEVQLVESGGGLVQPGGSLRLSCAASGFTFSTYAMNWVRQA
PGKGLEWVGRIRSKYNNYATYYADSVKGRFTISRDDSKNTLY
LQMNSLRAEDTAVYYCVRHGNFGDSYVSWFAYWGQGTLVT
VSSGKPGSGKPGSGKPGSGKPGSQAVVTQEPSLTVSPGGTVTLT
CGSSTGAVTTSNYANWVQQKPGKSPRGLIGGTNKRAPGVPA
RFSGSLLGGKAALTISGAQPEDEADYYCALWYSNHWVFGGGT
KLTVL
>SEQ ID NO: 105
(SEQ ID NO: 105)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDAWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSEVQLVESGGGLVQPGGSLRLSCAASGFTFNTYAMNWVRQA
PGKGLEWVARIRSKYNNYATYYADSVKSRTTISDDSKNTLYL
QMNSLRAEDTAVYYCVRHGNFGNSYVSWFAYWGQTTVTVSS
GGGGSGGGGSGGGGSGGGGSEIVVTQSPATLSVSPGERATLSC
RSSTGAVTTSNYANWVQEPGQAFRGLIGGANKRAPGVPARFS
GSLSGDEATLTISSLQSEDFAVYYCALWYSNLWVFGQGTKLEI
K
>SEQ ID NO: 106
(SEQ ID NO: 106)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDVWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSEVQLVESGGGLVQPGGSLRLSCAASGFTFNTYAMNWVRQA
PGKGLEWVARIRSKYNNYATYYADSVKSRTTISDDSKNTLYL
QMNSLRAEDTAVYYCVRHGNFGNSYVSWFAYWGQTTVTVSS
GGGGSGGGGSGGGGSGGGGSEIVVTQSPATLSVSPGERATLSC
RSSTGAVTTSNYANWVQEPGQAFRGLIGGANKRAPGVPARFS
GSLSGDEATLTISSLQSEDFAVYYCALWYSNLWVFGQGTKLEI
K
>SEQ ID NO: 107
(SEQ ID NO: 107)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDAWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSEVQLVESGGGLVQPGGSLKLSCAASGFTFNKYAMNWVRQ
APGKGLEWVARIRSKYNNYATYYADSVKDRFTISRDDSKNTA
YLQMNNLKTEDTAVYYCVRHGNFGNSYISYWAYWGQGTLV
TVSSGGGGSGGGGSGGGGSQTVVTQEPSLTVSPGGTVTLTCGS
STGAVTSGNYPNWVQQKPGQAPRGLIGGTKFLAPGTPARFSG
SLLGGKAALTLSGVQPEDEAEYYCVLWYSNRWVFGGGTKLT
VL
>SEQ ID NO: 109
(SEQ ID NO: 109)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDVWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSEVQLVESGGGLVQPGGSLKLSCAASGFTFNKYAMNWVRQ
APGKGLEWVARIRSKYNNYATYYADSVKDRFTISRDDSKNTA
YLQMNNLKTEDTAVYYCVRHGNFGNSYISYWAYWGQGTLV
TVSSGGGGSGGGGSGGGGSQTVVTQEPSLTVSPGGTVTLTCGS
STGAVTSGNYPNWVQQKPGQAPRGLIGGTKFLAPGTPARFSG
SLLGGKAALTLSGVQPEDEAEYYCVLWYSNRWVFGGGTKLT
VL
>SEQ ID NO: 112
(SEQ ID NO: 112)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDAWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSEVQLLESGGGLVQPGGSLRLSCAASGFSFTGYTMNWVRQA
PGKGLEWMGLINPYKGVSTYNQKVKGRFTISRDNSKNTLYLQ
MNSLRAEDTAVYYCARSGYYGDSDWYFDVWGQGTLVTVSS
GGGGSGGGGSGGGGSDIQMTQSPSSLSASVGDRVTITCRASQD
IRNYLNWYQQTPGKAPKRWIYYTSRLHSGVPSRFSGSGSGTD
YTFTYSSLQPEDIATYYCQQGNTLPWTFGQGTKLEIK
>SEQ ID NO: 113
(SEQ ID NO: 113)
EVQLLESGGGLVQPGGSLRLSCAASGFTFSSYAMNWVRQAPG
KGLEWVAGISSSGRYTGYADSVKGRFTISRDNSKNTLYLQMN
SLRAEDTAVYYCAKSVGSGVSWSGYVATSLDVWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSRDELTKNQVSLTCLVKGFYPS
DIAVEWESNGQPENNYKTTPPVLDSDGSFFLYSKLTVDKSRWQ
QGNVFSCSVMHEALHNHYTQKSLSLSPGKGGGGSGGGGTGGG
GSEVQLLESGGGLVQPGGSLRLSCAASGFSFTGYTMNWVRQA
PGKGLEWMGLINPYKGVSTYNQKVKGRFTISRDNSKNTLYLQ
MNSLRAEDTAVYYCARSGYYGDSDWYFDVWGQGTLVTVSS
GGGGSGGGGSGGGGSDIQMTQSPSSLSASVGDRVTITCRASQD
IRNYLNWYQQTPGKAPKRWIYYTSRLHSGVPSRFSGSGSGTD
YTFTYSSLQPEDIATYYCQQGNTLPWTFGQGTKLEIK
>SEQ ID NO: 81
(SEQ ID NO: 81)
EVQLLESGGGLVQPGGSLRLSCAASGFTYRGYSMGWVRQAPG
KGLEFVAAIVWSGGNTYYEDSVKGRFTISRDNSKNTLYLQMN
SLRAEDTAVYYCAAKIRPYIFKIAGQYDYWGQGTLVTVSSAS
TKGPSVFPLAPSSKSTSGGTAALGCLVKDYFPEPVTVSWNSGA
LTSGVHTFPAVLQSSGLYSLSSVVTVPSSSLGTQTYICNVNHKP
SNTKVDKKVEPKSCDKTHTCPPCPAPEAAGGPSVFLFPPKPKDT
LMISRTPEVTCVVVDVSHEDPEVKFNWYVDGVEVHNAKTKPR
EEQYNSTYRVVSVLTVLHQDWLNGKEYKCKVSNKALPAPIEK
TISKAKGQPREPQVYTLPPSREEMTKNQVSLSCAVKGFYPSDIA
VEWESNGQPENNYKTTPPVLDSDGSFFLVSKLTVDKSRWQQG
NVFSCSVMHEALHNHYTQKSLSLSPGK
>SEQ ID NO: 121
(SEQ ID NO: 121)
EVQLVESGGGLVQPGGSLRLSCAASGFTFSTYAMNWVRQAPG
KGLEWVGRIRSKYNNYATYYADSVKGRFTISRDDSKNTLYLQ
MNSLRAEDTAVYYCVRHGNFGDSYVSWFAYWGQGTLVTVSS
ASTKGPSVFPLAPSSKSTSGGTAALGCLVKDYFPEPVTVSWNSG
ALTSGVHTFPAVLQSSGLYSLSSVVTVPSSSLGTQTYICNVNHK
PSNTKVDKKVEPKSCDKTHTCPPCPAPEAAGGPSVFLFPPKPKD
TLMISRTPEVTCVVVDVSHEDPEVKFNWYVDGVEVHNAKTKP
REEQYNSTYRVVSVLTVLHQDWLNGKEYKCKVSNKALPAPIE
KTISKAKGQPREPQVYTLPPSREEMTKNQVSLSCAVKGFYPSDI
AVEWESNGQPENNYKTTPPVLDSDGSFFLVSKLTVDKSRWQQ
GNVFSCSVMHEALHNHYTQKSLSLSPGK
>SEQ ID NO: 123
(SEQ ID NO: 123)
EVQLVESGGGLVQPGGSLRLSCAASGFTFNTYAMNWVRQAPG
KGLEWVARIRSKYNNYATYYADSVKSRTTISDDSKNTLYLQM
NSLRAEDTAVYYCVRHGNFGNSYVSWFAYWGQTTVTVSSAS
TKGPSVFPLAPSSKSTSGGTAALGCLVKDYFPEPVTVSWNSGA
LTSGVHTFPAVLQSSGLYSLSSVVTVPSSSLGTQTYICNVNHKP
SNTKVDKKVEPKSCDKTHTCPPCPAPEAAGGPSVFLFPPKPKDT
LMISRTPEVTCVVVDVSHEDPEVKFNWYVDGVEVHNAKTKPR
EEQYNSTYRVVSVLTVLHQDWLNGKEYKCKVSNKALPAPIEK
TISKAKGQPREPQVYTLPPSREEMTKNQVSLSCAVKGFYPSDIA
VEWESNGQPENNYKTTPPVLDSDGSFFLVSKLTVDKSRWQQG
NVFSCSVMHEALHNHYTQKSLSLSPGK
>SEQ ID NO: 125
(SEQ ID NO: 125)
EVQLVESGGGLVQPGGSLKLSCAASGFTFNKYAMNWVRQAP
GKGLEWVARIRSKYNNYATYYADSVKDRFTISRDDSKNTAYL
QMNNLKTEDTAVYYCVRHGNFGNSYISYWAYWGQGTLVTV
SSASTKGPSVFPLAPSSKSTSGGTAALGCLVKDYFPEPVTVSWN
SGALTSGVHTFPAVLQSSGLYSLSSVVTVPSSSLGTQTYICNVN
HKPSNTKVDKKVEPKSCDKTHTCPPCPAPEAAGGPSVFLFPPKP
KDTLMISRTPEVTCVVVDVSHEDPEVKFNWYVDGVEVHNAKT
KPREEQYNSTYRVVSVLTVLHQDWLNGKEYKCKVSNKALPAP
IEKTISKAKGQPREPQVYTLPPSREEMTKNQVSLSCAVKGFYPS
DIAVEWESNGQPENNYKTTPPVLDSDGSFFLVSKLTVDKSRWQ
QGNVFSCSVMHEALHNHYTQKSLSLSPGK
>SEQ ID NO: 127
(SEQ ID NO: 127)
QVQLVQSGGGVVQPGRSLRLSCKASGYTFTRYTMHWVRQAP
GKGLEWIGYINPSRGYTNYNQKVKDRFTISTDKSKSTAFLQM
DSLRPEDTAVYYCARYYDDHYCLDYWGQGTTLTVSSASTKGP
SVFPLAPSSKSTSGGTAALGCLVKDYFPEPVTVSWNSGALTSG
VHTFPAVLQSSGLYSLSSVVTVPSSSLGTQTYICNVNHKPSNTK
VDKKVEPKSCDKTHTCPPCPAPEAAGGPSVFLFPPKPKDTLMIS
RTPEVTCVVVDVSHEDPEVKFNWYVDGVEVHNAKTKPREEQY
NSTYRVVSVLTVLHQDWLNGKEYKCKVSNKALPAPIEKTISKA
KGQPREPQVYTLPPSREEMTKNQVSLSCAVKGFYPSDIAVEWE
SNGQPENNYKTTPPVLDSDGSFFLVSKLTVDKSRWQQGNVFSC
SVMHEALHNHYTQKSLSLSPGK
>SEQ ID NO: 129
(SEQ ID NO: 129)
EVQLLESGGGLVQPGGSLRLSCAASGFSFTGYTMNWVRQAPG
KGLEWMGLINPYKGVSTYNQKVKGRFTISRDNSKNTLYLQM
NSLRAEDTAVYYCARSGYYGDSDWYFDVWGQGTLVTVSSAS
TKGPSVFPLAPSSKSTSGGTAALGCLVKDYFPEPVTVSWNSGA
LTSGVHTFPAVLQSSGLYSLSSVVTVPSSSLGTQTYICNVNHKP
SNTKVDKKVEPKSCDKTHTCPPCPAPEAAGGPSVFLFPPKPKDT
LMISRTPEVTCVVVDVSHEDPEVKFNWYVDGVEVHNAKTKPR
EEQYNSTYRVVSVLTVLHQDWLNGKEYKCKVSNKALPAPIEK
TISKAKGQPREPQVYTLPPSREEMTKNQVSLSCAVKGFYPSDIA
VEWESNGQPENNYKTTPPVLDSDGSFFLVSKLTVDKSRWQQG
NVFSCSVMHEALHNHYTQKSLSLSPGK
>SEQ ID NO: 122
(SEQ ID NO: 122)
QAVVTQEPSLTVSPGGTVTLTCGSSTGAVTTSNYANWVQQKP
GKSPRGLIGGTNKRAPGVPARFSGSLLGGKAALTISGAQPEDE
ADYYCALWYSNHWVFGGGTKLTVLGQPKAAPSVTLFPPSSEE
LQANKATLVCLISDFYPGAVTVAWKADSSPVKAGVETTTPSKQ
SNNKYAASSYLSLTPEQWKSHRSYSCQVTHEGSTVEKTVAPTE
CS
>SEQ ID NO: 124
(SEQ ID NO: 124)
EIVVTQSPATLSVSPGERATLSCRSSTGAVTTSNYANWVQEPG
QAFRGLIGGANKRAPGVPARFSGSLSGDEATLTISSLQSEDFAV
YYCALWYSNLWVFGQGTKLEIKTVAAPSVFIFPPSDEQLKSGT
ASVVCLLNNFYPREAKVQWKVDNALQSGNSQESVTEQDSKDS
TYSLSSTLTLSKADYEKHKVYACEVTHQGLSSPVTKSFNRGEC
>SEQ ID NO: 126
(SEQ ID NO: 126)
EQTVVTQEPSLTVSPGGTVTLTCGSSTGAVTSGNYPNWVQQK
PGQAPRGLIGGTKFLAPGTPARFSGSLLGGKAALTLSGVQPED
EAEYYCVLWYSNRWVFGGGTKLTVLGQPKAAPSVTLFPPSSE
ELQANKATLVCLISDFYPGAVTVAWKADSSPVKAGVETTTPSK
QSNNKYAASSYLSLTPEQWKSHRSYSCQVTHEGSTVEKTVAPT
ECS
>SEQ ID NO: 128
(SEQ ID NO: 128)
DIQMTQSPSSLSASVGDRVTITCSASSSVSYMNWYQQTPGKAP
KRWIYDTSKLASGVPSRFSGSGSGTDYTFTISSLQPEDIATYYC
QQWSSNPFTFGQGTKLQITRRTVAAPSVFIFPPSDEQLKSGTAS
VVCLLNNFYPREAKVQWKVDNALQSGNSQESVTEQDSKDSTY
SLSSTLTLSKADYEKHKVYACEVTHQGLSSPVTKSFNRGEC
>SEQ ID NO: 130
(SEQ ID NO: 130)
DIQMTQSPSSLSASVGDRVTITCRASQDIRNYLNWYQQTPGKA
PKRWIYYTSRLHSGVPSRFSGSGSGTDYTFTYSSLQPEDIATYY
CQQGNTLPWTFGQGTKLEIKTVAAPSVFIFPPSDEQLKSGTAS
VVCLLNNFYPREAKVQWKVDNALQSGNSQESVTEQDSKDSTY
SLSSTLTLSKADYEKHKVYACEVTHQGLSSPVTKSFNRGEC
>SEQ ID NO: 117
(SEQ ID NO: 117)
EVQLLESGGGLVQPGGSLRLSCAASGFTFSSYAMNWVRQAPG
KGLEWVAGISSSGRYTGYADSVKGRFTISRDNSKNTLYLQMNS
LRAEDTAVYYCAKSVGSGVSWSGYVATSLDAWGQGTLVTVS
SASTKGPSVFPLAPSSKSTSGGTAALGCLVKDYFPEPVTVSWNS
GALTSGVHTFPAVLQSSGLYSLSSVVTVPSSSLGTQTYICNVNH
KPSNTKVDKKVEPKSCDKTHTCPPCPAPEAAGGPSVFLFPPKPK
DTLMISRTPEVTCVVVDVSHEDPEVKFNWYVDGVEVHNAKTK
PREEQYNSTYRVVSVLTVLHQDWLNGKEYKCKVSNKALPAPI
EKTISKAKGQPREPQVYTLPPSREEMTKNQVSLSCAVKGFYPSD
IAVEWESNGQPENNYKTTPPVLDSDGSFFLVSKLTVDKSRWQQ
GNVFSCSVMHEALHNHYTQKSLSLSPGKGGGGSGGGGTGGGG
SEVQLVESGGGPVQAGGSLRLSCAASGRTYRGYSMGWFRQAP
GKEREFVAAIVWSGGNTYYEDSVKGRFTISRDNAKNTMYLQM
TSLKPEDSATYYCAAKIRPYIFKIAGQYDYWGQGTQVTVSS
>SEQ ID NO: 119
(SEQ ID NO: 119)
EVQLLESGGGLVQPGGSLRLSCAASGFTFSSYAMNWVRQAPG
KGLEWVAGISSSGRYTGYADSVKGRFTISRDNSKNTLYLQMNS
LRAEDTAVYYCAKSVGSGVSWSGYVATSLDVWGQGTLVTVS
SASTKGPSVFPLAPSSKSTSGGTAALGCLVKDYFPEPVTVSWNS
GALTSGVHTFPAVLQSSGLYSLSSVVTVPSSSLGTQTYICNVNH
KPSNTKVDKKVEPKSCDKTHTCPPCPAPEAAGGPSVFLFPPKPK
DTLMISRTPEVTCVVVDVSHEDPEVKFNWYVDGVEVHNAKTK
PREEQYNSTYRVVSVLTVLHQDWLNGKEYKCKVSNKALPAPI
EKTISKAKGQPREPQVYTLPPSREEMTKNQVSLSCAVKGFYPSD
IAVEWESNGQPENNYKTTPPVLDSDGSFFLVSKLTVDKSRWQQ
GNVFSCSVMHEALHNHYTQKSLSLSPGKGGGGGGGGTGGGG
SEVQLVESGGGPVQAGGSLRLSCAASGRTYRGYSMGWFRQAP
GKEREFVAAIVWSGGNTYYEDSVKGRFTISRDNAKNTMYLQM
TSLKPEDSATYYCAAKIRPYIFKIAGQYDYWGQGTQVTVSS
>SEQ ID NO: 118
(SEQ ID NO: 118)
EVQLVESGGGPVQAGGSLRLSCAASGRTYRGYSMGWFRQAPG
KEREFVAAIVWSGGNTYYEDSVKGRFTISRDNAKNTMYLQMT
SLKPEDSATYYCAAKIRPYIFKIAGQYDYWGQGTQVTVSSGGG
GSGGGGTGGGGSEVQLLESGGGLVQPGGSLRLSCAASGFTFSS
YAMNWVRQAPGKGLEWVAGISSSGRYTGYADSVKGRFTISRD
NSKNTLYLQMNSLRAEDTAVYYCAKSVGSGVSWSGYVATSLD
AWGQGTLVTVSSASTKGPSVFPLAPSSKSTSGGTAALGCLVKD
YFPEPVTVSWNSGALTSGVHTFPAVLQSSGLYSLSSVVTVPSSS
LGTQTYICNVNHKPSNTKVDKKVEPKSCDKTHTCPPCPAPEAA
GGPSVFLFPPKPKDTLMISRTPEVTCVVVDVSHEDPEVKFNWY
VDGVEVHNAKTKPREEQYNSTYRVVSVLTVLHQDWLNGKEY
KCKVSNKALPAPIEKTISKAKGQPREPQVYTLPPSREEMTKNQV
SLSCAVKGFYPSDIAVEWESNGQPENNYKTTPPVLDSDGSFFLV
SKLTVDKSRWQQGNVFSCSVMHEALHNHYTQKSLSLSPGK
>SEQ ID NO: 120
(SEQ ID NO: 120)
EVQLVESGGGPVQAGGSLRLSCAASGRTYRGYSMGWFRQAPG
KEREFVAAIVWSGGNTYYEDSVKGRFTISRDNAKNTMYLQMT
SLKPEDSATYYCAAKIRPYIFKIAGQYDYWGQGTQVTVSSGGG
GSGGGGTGGGGSEVQLLESGGGLVQPGGSLRLSCAASGFTFSS
YAMNWVRQAPGKGLEWVAGISSSGRYTGYADSVKGRFTISRD
NSKNTLYLQMNSLRAEDTAVYYCAKSVGSGVSWSGYVATSLD
VWGQGTLVTVSSASTKGPSVFPLAPSSKSTSGGTAALGCLVKD
YFPEPVTVSWNSGALTSGVHTFPAVLQSSGLYSLSSVVTVPSSS
LGTQTYICNVNHKPSNTKVDKKVEPKSCDKTHTCPPCPAPEAA
GGPSVFLFPPKPKDTLMISRTPEVTCVVVDVSHEDPEVKFNWY
VDGVEVHNAKTKPREEQYNSTYRVVSVLTVLHQDWLNGKEY
KCKVSNKALPAPIEKTISKAKGQPREPQVYTLPPSREEMTKNQV
SLSCAVKGFYPSDIAVEWESNGQPENNYKTTPPVLDSDGSFFLV
SKLTVDKSRWQQGNVFSCSVMHEALHNHYTQKSLSLSPGK

In some embodiments, the antibody specific for claudin 6 and CD3 (e.g. a tandem scFv antibody, scFv-Fab Fc antibody, IgG-(scFV)₂ antibody, and a scFv-Fab Fe antibody), or fragment thereof, or variant thereof, is selected from one of the following amino acid sequences:

>CH-HAMF5-1HEP-VH (SEQ ID NO: 69)
(SEQ ID NO: 69)
EVQLLESGGGLVQPGGSLRLSCAASGFTFSSYAMNWVRQAPG
KGLEWVAGISSSGRYTGYADSVKGRFTISRDNSKNTLYLQMNS
LRAEDTAVYYCAKSVGSGVSWSGYVATSLDAWGQGTLVTVSS
>CH-HAMF5-1HEP-VL (SEQ ID NO: 68)
(SEQ ID NO: 68)
SYELTQPPSVSVSPGQTARITCSAGSGLYGWYQQKPGQAPVLVI
YGTNKRPSGIPERFSGSSSGTTVTLTISGVQAEDEADYYCGSAD
SSTNAGIFGGGTKLTVL
>CD3engager (SEQ ID NO: 75)
(SEQ ID NO: 75)
EVQLVESGGGPVQAGGSLRLSCAASGRTYRGYSMGWFRQAPG
KEREFVAAIVWSGGNTYYEDSVKGRFTISRDNAKNTMYLQMT
SLKPEDSATYYCAAKIRPYIFKIAGQYDYWGQGTQVTVSS

In some embodiments, the antibody specific for claudin 6 and CD3 (e.g. a tandem scFv antibody, scFv-Fab Fc antibody, IgG-(scFV)₂ antibody, and a scFv-Fab Fe antibody), or fragment thereof, or variant thereof, is selected from one of the following amino acid sequences:

>CH-HAMF5-1HEP-HC (SEQ ID NO: 76)
(SEQ ID NO: 76)
EVQLLESGGGLVQPGGSLRLSCAASGFTFSSYAMNWVRQAPG
KGLEWVAGISSSGRYTGYADSVKGRFTISRDNSKNTLYLQMNS
LRAEDTAVYYCAKSVGSGVSWSGYVATSLDAWGQGTLVTVS
SASTKGPSVFPLAPSSKSTSGGTAALGCLVKDYFPEPVTVSWNS
GALTSGVHTFPAVLQSSGLYSLSSVVTVPSSSLGTQTYICNVNH
KPSNTKVDKKVEPKSCDKTHTCPPCPAPEAAGGPSVFLFPPKPK
DTLMISRTPEVTCVVVDVSHEDPEVKFNWYVDGVEVHNAKTK
PREEQYNSTYRVVSVLTVLHQDWLNGKEYKCKVSNKALPAPI
EKTISKAKGQPREPQVYTLPPSREEMTKNQVSLWCLVKGFYPS
DIAVEWESNGQPENNYKTTPPVLDSDGSFFLYSKLTVDKSRWQ
QGNVFSCSVMHEALHNHYTQKSLSLSPGK
>CH-HAMF5-1HEP-LC (SEQ ID NO: 77)
(SEQ ID NO: 77)
SYELTQPPSVSVSPGQTARITCSAGSGLYGWYQQKPGQAPVLVI
YGTNKRPSGIPERFSGSSSGTTVTLTISGVQAEDEADYYCGSAD
SSTNAGIFGGGTKLTVLGQPKAAPSVTLFPPSSEELQANKATLV
CLISDFYPGAVTVAWKADSSPVKAGVETTTPSKQSNNKYAASS
YLSLTPEQWKSHRSYSCQVTHEGSTVEKTVAPTECS
>3983-HC (SEQ ID NO: 78)
(SEQ ID NO: 78)
EVQLVESGGGPVQAGGSLRLSCAASGRTYRGYSMGWFRQAPG
KEREFVAAIVWSGGNTYYEDSVKGRFTISRDNAKNTMYLQMT
SLKPEDSATYYCAAKIRPYIFKIAGQYDYWGQGTQVTVSSASP
KSSDKTHTCPPCPAPEAAGGPSVFLFPPKPKDTLMISRTPEVTCV
VVDVSHEDPEVKFNWYVDGVEVHNAKTKPREEQYNSTYRVV
SVLTVLHQDWLNGKEYKCKVSNKALPAPIEKTISKAKGQPREP
QVYTLPPSREEMTKNQVSLSCAVKGFYPSDIAVEWESNGQPEN
NYKTTPPVLDSDGSFFLVSKLTVDKSRWQQGNVFSCSVMHEA
LHNHYTQKSLSLSPGK

In some embodiments, the antibody or antibody fragment or variant disclosed herein (e.g., a tandem scFv antibody, scFv-Fab Fc antibody, IgG-(scFV)2 antibody, and a scFv-Fab Fe antibody) comprises a CD3 engager. In some embodiments, the CD3 engager can be selected from muOKT3, huOKT3, huSP34, huUCHT1 and a CD3 nanobody (VHH).

In some embodiments, the antibody or antibody fragment or variant disclosed herein (e.g., a tandem scFv antibody, scFv-Fab Fc antibody, IgG-(scFV)2 antibody, and a scFv-Fab Fc antibody) comprise a linker having one or more glycines and serines replaced with a functionally equivalent variation thereof. In some embodiments, the linker is identified in the underlined text above. For example, in some embodiments, the linker is selected from the amino acid sequence of GGGGSGGGGSGGGGS (SEQ ID NO: 50), GGGGS (SEQ ID NO: 51), GGSGGSGGSGGSGGVD (SEQ ID NO: 52), and GKPGSGKPGSGKPGSGKPGS (SEQ ID NO: 53).

In some embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid. In various embodiments, the linker is substantially comprised of glycine and serine residues (e.g. about 30%, or about 40%, or about 50%, or about 60%, or about 70%, or about 80%, or about 90%, or about 95%, or about 97% glycines and serines).

Additional illustrative linkers include, but are not limited to, linkers having the sequence LE, GGGGS (SEQ ID NO: 51), (GGGGS)ₙ (n=1-4) (SEQ ID NO: 54), (Gly)₈ (SEQ ID NO: 55), (Gly)₆ (SEQ ID NO: 56), (EAAAK)ₙ (n=1-3) (SEQ ID NO: 57), A(EAAAK)ₙA (n=2-5) (SEQ ID NO: 58), AEAAAKEAAAKA (SEQ ID NO: 59), A(EAAAK)₄ALEA(EAAAK)₄A (SEQ ID NO: 60), PAPAP (SEQ ID NO: 61), KESGSVSSEQLAQFRSLD (SEQ ID NO: 62), EGKSSGSGSESKST(SEQ ID NO: 63), GSAGSAAGSGEF (SEQ ID NO: 64), and (XP)ₙ (n=1-5) (SEQ ID NO: 131), with X designating any amino acid, e.g., Ala, Lys, or Glu.

In various embodiments, the linker may be functional. For example, without limitation, the linker may function to improve the folding and/or stability, improve the expression, improve the pharmacokinetics, and/or improve the bioactivity of the present compositions. In another example, the linker may function to target the compositions to a particular cell type or location.

In some embodiments, disclosed herein is a composition comprising a tandem single-chain variable fragment (scFv) specific for claudin 6 and CD3 comprising one or more of:
I.
(a) an amino acid sequence of SEQ ID NO. 82, or an amino acid sequence having at least about 90%, or about 93%, or about 95%, or about 97%, or about 98%, or about 99% identity thereto, or
(b)
   (i) a light chain variable region (VL) comprising LCDR1, LCDR2, and LCDR3 sequences, wherein the LCDR1 comprises an amino acid sequence of SAGSGLYG (SEQ ID NO: 1), or a variant thereof, the lCDR2 sequence comprises an amino acid sequence of GTNKRPS (SEQ ID NO: 2), or a variant thereof, and the LCDR3 sequence comprises an amino acid sequence of GSADSSTNAGI (SEQ ID NO: 3), or a variant thereof,
   (ii) a heavy chain variable region (VH) comprising HCDR1, HCDR2, and HCDR3 sequences, wherein the HCDR1 comprises an amino acid sequence of SYAMN (SEQ ID NO: 4) or a variant thereof, the HCDR2 comprises an amino acid sequence of GISSSGRYTGYADSVKG (SEQ ID NO: 5), or a variant thereof, and the HCDR3 comprises an amino acid sequence of SVGSGVSWSGYVATSLDA (SEQ ID NO: 6), or a variant thereof,
   (iii) a VH comprising HCDR1, HCDR2, and HCDR3 sequences, wherein the HCDR1 comprises an amino acid sequence of RYTMH (SEQ ID NO: 7), or a variant thereof, the HCDR2 comprises an amino acid sequence of YINPSRGYTNYNQKFKD (SEQ ID NO: 8), or a variant thereof, and the HCDR3 comprises an amino acid sequence of YYDDHYCLDY (SEQ ID NO: 9), or a variant thereof, and
   (iv) a VL comprising LCDR1, LCDR2, and LCDR3 sequences, wherein the LCDR1 comprises an amino acid sequence of RASSSVSYMN (SEQ ID NO: 10), or a variant thereof, the LCDR2 comprises an amino acid sequence of DTSKVAS (SEQ ID NO: 11), or a variant thereof, and the LCDR3 comprises an amino acid sequence of QQWSSNPLT (SEQ ID NO: 12), or a variant thereof,
II.
(a) an amino acid sequence of SEQ ID NO. 83, or an amino acid sequence having at least about 90%, or about 93%, or about 95%, or about 97%, or about 98%, or about 99% identity thereto,
or
(b)
   (i) a VL comprising LCDR1, LCDR2, and LCDR3 sequences, wherein the LCDR1 comprises an amino acid sequence of SGGSGSYG (SEQ ID NO: 13), or a variant thereof, the LCDR2 comprises an amino acid sequence of GTYKRPS (SEQ ID NO: 14), or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence is SEQ ID NO: 4, or a variant thereof, the HCDR2 comprises an amino acid sequence of SEQ ID NO: 5, or a variant thereof, and the HCDR3 comprises an amino acid sequence of SVGSGVSWSGYVATSLDV (SEQ ID NO: 15), or a variant thereof,
   (iii) a VH comprising HCDR1, HCDR2, and HCDR3 sequences, wherein the HCDR1 comprises an amino acid sequence of SEQ ID NO: 7, or a variant thereof, the HCDR2 comprises an amino acid sequence of SEQ ID NO: 8, or a variant thereof, and the HCDR3 comprises an amino acid sequence of SEQ ID NO: 9, or a variant thereof, and
   (iv) a VL comprising LCDR1, LCDR2, and LCDR3 sequences, wherein the LCDR1 comprises an amino acid sequence of SEQ ID NO: 10, or a variant thereof, the LCDR2 comprises an amino acid sequence of SEQ ID NO: 11, or a variant thereof, and the LCDR3 comprises an amino acid sequence of SEQ ID NO: 12, or a variant thereof,
III.
(a) an amino acid sequence of SEQ ID NO. 84, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
      (ii) a VH comprising HCDR1, HCDR2, and HCDR3 sequences, wherein the HCDR1 comprises an amino acid sequence of SEQ ID NO: 4, or a variant thereof, the HCDR2 sequence of SEQ ID NO: 5, or a variant thereof, and the HCDR3 sequence of SEQ ID NO: 6, or a variant thereof, and
      (iii) a VH comprising HCDR1, HCDR2, and HCDR3 sequences, wherein the HCDR1 comprises an amino acid sequence of GYSMG (SEQ ID NO: 16), or a variant thereof, the HCDR2 comprises an amino acid sequence of AIVWSGGNTYYEDSVKG (SEQ ID NO: 17), or a variant thereof, and the HCDR3 comprises an amino acid sequence of KIRPYIFKIAGQYDY (SEQ ID NO: 18), or a variant thereof,
IV.
(a) an amino acid sequence of SEQ ID NO: 85, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof, and
   (iii) a VH comprising HCDR1, HCDR2, and HCDR3 sequences, wherein the HCDR1 comprises an amino acid sequence of SEQ ID NO: 16, or a variant thereof, the HCDR2 comprises an amino acid sequence of SEQ ID NO: 17, or a variant thereof, and the HCDR3 comprises an amino acid sequence of SEQ ID NO: 18, or a variant thereof,
V.
(a) an amino acid sequence of SEQ ID NO. 93, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of KYAMN (SEQ ID NO: 19), or a variant thereof, the HCDR2 comprises an amino acid sequence of RIRSKYNNYATYYADSVKD (SEQ ID NO: 20), or a variant thereof, and the HCDR3 comprises an amino acid sequence of HGNFGNSYISYWAY (SEQ ID NO: 21), or a variant thereof, and
   (iv) a VL comprising LCDR1, LCDR2, and LCDR3 sequences, wherein the LCDR1 comprises an amino acid sequence of GSSTGAVTSGNYPN (SEQ ID NO: 22), or a variant thereof, the LCDR2 comprises an amino acid sequence of GTKFLAP (SEQ ID NO: 23), or a variant thereof, and the LCDR3 comprises an amino acid sequence of VLWYSNRWV (SEQ ID NO: 24), or a variant thereof,
VI.
(a) an amino acid sequence of SEQ ID NO. 94, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 19, or a variant thereof, the HCDR2 comprises an amino acid sequence of SEQ ID NO: 20, or a variant thereof, and the HCDR3 comprises an amino acid sequence of SEQ ID NO: 21, or a variant thereof, and
   (iv) a VL comprising LCDR1, LCDR2, and LCDR3 sequences, wherein the LCDR1 comprises an amino acid sequence of SEQ ID NO: 22, or a variant thereof, the LCDR2 comprises an amino acid sequence of SEQ ID NO: 23, or a variant thereof, and the LCDR3 comprises an amino acid sequence of SEQ ID NO: 24, or a variant thereof,
VII.
(a) an amino acid sequence of SEQ ID NO. 95, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of TYAMN (SEQ ID NO: 25), or a variant thereof, the HCDR2 comprises an amino acid sequence of RIRSKYNNYATYYADSVKG (SEQ ID NO: 26), or a variant thereof, and the HCDR3 comprises an amino acid sequence of HGNFGDSYVSWFAY (SEQ ID NO: 27), or a variant thereof, and
   (iv) a VL comprising LCDR1, LCDR2, and LCDR3 sequences, wherein the LCDR1 comprises an amino acid sequence of GSSTGAVTTSNYAN (SEQ ID NO: 28), or a variant thereof, the LCDR2 comprises an amino acid sequence of GTNKRAP (SEQ ID NO: 29), or a variant thereof, and the LCDR3 comprises an amino acid sequence of ALWYSNHWV (SEQ ID NO: 30), or a variant thereof,
VIII.
(a) an amino acid sequence of SEQ ID NO. 96, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 25, or a variant thereof, the HCDR2 comprises an amino acid sequence of SEQ ID NO: 26, or a variant thereof, and the HCDR3 comprises an amino acid sequence of SEQ ID NO: 27, or a variant thereof, and
   (iv) a VL comprising LCDR1, LCDR2, and LCDR3 sequences, wherein the LCDR1 comprises an amino acid sequence of SEQ ID NO: 28, or a variant thereof, the LCDR2 comprises an amino acid sequence of SEQ ID NO: 29, or a variant thereof, and the LCDR3 comprises an amino acid sequence of SEQ ID NO: 30, or a variant thereof,
IX.
(a) an amino acid sequence of SEQ ID NO. 97, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of SEQ ID NO: 25, or a variant thereof, the HCDR2 comprises an amino acid sequence of RIRSKYNNYATYYADSVKS (SEQ ID NO: 31), or a variant thereof, and the HCDR3 comprises an amino acid sequence of HGNFGNSYVSWFAY (SEQ ID NO: 32), or a variant thereof, and
   (iv) a VL comprising LCDR1, LCDR2, and LCDR3 sequences, wherein the LCDR1 comprises an amino acid sequence of RSSTGAVTTSNYAN (SEQ ID NO: 33), or a variant thereof, the LCDR2 comprises an amino acid sequence of GANKRAP (SEQ ID NO: 34), or a variant thereof, and the LCDR3 comprises an amino acid sequence of ALWYSNLWV (SEQ ID NO: 35), or a variant thereof,
X.
(a) an amino acid sequence of SEQ ID NO. 98, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 25, or a variant thereof, the HCDR2 comprises an amino acid sequence of SEQ ID NO: 31, or a variant thereof, and the HCDR3 comprises an amino acid sequence of SEQ ID NO: 32, or a variant thereof, and
   (iv) a VL comprising LCDR1, LCDR2, and LCDR3 sequences, wherein the LCDR1 comprises an amino acid sequence of SEQ ID NO: 33, or a variant thereof, the LCDR2 comprises an amino acid sequence of SEQ ID NO: 34, or a variant thereof, and the LCDR3 comprises an amino acid sequence of SEQ ID NO: 35, or a variant thereof,
XI.
(a) an amino acid sequence of SEQ ID NO. 99, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of SEQ ID NO: 7, or a variant thereof, the HCDR2 comprises an amino acid sequence of YINPSRGYTNYNQKVKD (SEQ ID NO: 36), or a variant thereof, and the HCDR3 comprises an amino acid sequence of SEQ ID NO: 9, or a variant thereof, and
   (iv) a VL comprising LCDR1, LCDR2, and LCDR3 sequences, wherein the LCDR1 comprises an amino acid sequence of SASSSVSYMN (SEQ ID NO: 37), or a variant thereof, the LCDR2 comprises an amino acid sequence of DTSKLAS (SEQ ID NO: 38), or a variant thereof, and the LCDR3 comprises an amino acid sequence of QQWSSNPFT (SEQ ID NO: 39), or a variant thereof,
XII.
(a) an amino acid sequence of SEQ ID NO. 100, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 7, or a variant thereof, the HCDR2 comprises an amino acid sequence of SEQ ID NO: 36, or a variant thereof, and the HCDR3 comprises an amino acid sequence of SEQ ID NO: 9, or a variant thereof, and
   (iv) a VL comprising LCDR1, LCDR2, and LCDR3 sequences, wherein the LCDR1 comprises an amino acid sequence of SEQ ID NO: 37, or a variant thereof, the LCDR2 comprises an amino acid sequence of SEQ ID NO: 38, or a variant thereof, and the LCDR3 comprises an amino acid sequence of SEQ ID NO: 39, or a variant thereof,
XIII.
(a) an amino acid sequence of SEQ ID NO: 109, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of GYTMN (SEQ ID NO: 40), or a variant thereof, the HCDR2 comprises an amino acid sequence of LINPYKGVSTYNQKVKG (SEQ ID NO: 41), or a variant thereof, and the HCDR3 comprises an amino acid sequence of SGYYGDSDWYFDV (SEQ ID NO: 42), or a variant thereof, and
   (iv) a VL comprising LCDR1, LCDR2, and LCDR3 sequences, wherein the LCDR1 comprises an amino acid sequence of RASQDIRNYLN (SEQ ID NO: 43), or a variant thereof, the LCDR2 comprises an amino acid sequence of YTSRLHS (SEQ ID NO: 44), or a variant thereof, and the LCDR3 comprises an amino acid sequence of QQGNTLPWT (SEQ ID NO: 45), or a variant thereof, and
XIV.
(a) an amino acid sequence of SEQ ID NO: 110, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4), or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 40, or a variant thereof, the HCDR2 comprises an amino acid sequence of SEQ ID NO: 41, or a variant thereof, and the HCDR3 comprises an amino acid sequence of SEQ ID NO: 42, or a variant thereof, and
   (iv) a VL comprising LCDR1, LCDR2, and LCDR3 sequences, wherein the LCDR1 comprises an amino acid sequence of SEQ ID NO: 43, or a variant thereof, the LCDR2 comprises an amino acid sequence of SEQ ID NO: 44, or a variant thereof, and the LCDR3 comprises an amino acid sequence of SEQ ID NO: 45, or a variant thereof, or
wherein:
   any of the above sequences in subsections (a) optionally can have a His6 tag (e.g., HHHHHH (SEQ ID NO: 46)) added to the N or C-terminus and
   any of the above sequences optionally have the linker comprising one or more glycines and serines replaced with another peptide linker or functionally equivalent variation thereof.

In some embodiments, the antibody, such as a tandem scFv antibody specific for claudin 6 and CD3 comprise the CDR sequences as shown in Table 1 below:

| Tandem scFv | Domain | CDR1 | CDR2 | CDR3 |
|---|---|---|---|---|
| SEQ ID NO: 82 | First VL | SEQ ID NO: 1 | SEQ ID NO: 2 | SEQ ID NO: 3 |
| | | SAGSGLYG | GTNKRPS | GSADSSTNAGI |
| | First VH | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | Second VH | SEQ ID NO: 7 | SEQ ID NO: 8 | SEQ ID NO: 9 |
| | | RYTMH | YINPSRGYTNYNQ | YYDDHYCLDY |
| | | | KFKD | |
| | Second VL | SEQ ID NO: 10 | SEQ ID NO: 11 | SEQ ID NO: 12 |
| | | RASSSVSYMN | DTSKVAS | QQWSSNPLT |
| SEQ ID NO: 83 | First VL | SEQ ID NO: 13 | SEQ ID NO: 14 | SEQ ID NO: 3 |
| | | SGGSGSYG | GTYKRPS | GSADSSTNAGI |
| | First VH | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | Second VH | SEQ ID NO: 7 | SEQ ID NO: 8 | SEQ ID NO: 9 |
| | | RYTMH | YINPSRGYTNYNQ | YYDDHYCLDY |
| | | | KFKD | |
| | Second VL | SEQ ID NO: 10 | SEQ ID NO: 11 | SEQ ID NO: 12 |
| | | RASSSVSYMN | DTSKVAS | QQWSSNPLT |
| SEQ ID NO: 84 | First VL | SEQ ID NO: 1 | SEQ ID NO: 2 | SEQ ID NO: 3 |
| | | SAGSGLYG | GTNKRPS | GSADSSTNAGI |
| | First VH | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | Second VH | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | Second VL | | | |
| SEQ ID NO: 85 | First VL | SEQ ID NO: 13 | SEQ ID NO: 14 | SEQ ID NO: 3 |
| | | SGGSGSYG | GTYKRPS | GSADSSTNAGI |
| | First VH | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | Second VH | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | Second VL | | | |
| SEQ ID NO: 93 | First VL | SEQ ID NO: 1 | SEQ ID NO: 2 | SEQ ID NO: 3 |
| | | SAGSGLYG | GTNKRPS | GSADSSTNAGI |
| | First VH | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | Second VH | SEQ ID NO: 19 | SEQ ID NO: 20 | SEQ ID NO: 21 |
| | | KYAMN | RIRSKYNNYATYY | HGNFGNSYISYWA |
| | | | ADSVKD | Y |
| | Second VL | SEQ ID NO: 22 | SEQ ID NO: 23 | SEQ ID NO: 24 |
| | | GSSTGAVTSGNYP | GTKFLAP | VLWYSNRWV |
| | | N | | |
| SEQ ID NO: 94 | First VL | SEQ ID NO: 13 | SEQ ID NO: 14 | SEQ ID NO: 3 |
| | | SGGSGSYG | GTYKRPS | GSADSSTNAGI |
| | First VH | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | Second VH | SEQ ID NO: 19 | SEQ ID NO: 20 | SEQ ID NO: 21 |
| | | KYAMN | RIRSKYNNYATYY | HGNFGNSYISYWA |
| | | | ADSVKD | Y |
| | Second VL | SEQ ID NO: 22 | SEQ ID NO: 23 | SEQ ID NO: 24 |
| | | GSSTGAVTSGNYP | GTKFLAP | VLWYSNRWV |
| | | N | | |
| SEQ ID NO: 95 | First VL | SEQ ID NO: 1 | SEQ ID NO: 2 | SEQ ID NO: 3 |
| | | SAGSGLYG | GTNKRPS | GSADSSTNAGI |
| | First VH | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | Second VH | SEQ ID NO: 25 | SEQ ID NO: 26 | SEQ ID NO: 27 |
| | | TYAMN | RIRSKYNNYATYY | HGNFGDSYVSWFA |
| | | | ADSVKG | Y |
| | Second VL | SEQ ID NO: 28 | SEQ ID NO: 29 | SEQ ID NO: 30 |
| | | GSSTGAVTTSNYA | GTNKRAP | ALWYSNHWV |
| | | N | | |
| SEQ ID NO: 96 | First VL | SEQ ID NO: 13 | SEQ ID NO: 14 | SEQ ID NO: 3 |
| | | SGGSGSYG | GTYKRPS | GSADSSTNAGI |
| | First VH | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | Second VH | SEQ ID NO: 25 | SEQ ID NO: 26 | SEQ ID NO: 27 |
| | | TYAMN | RIRSKYNNYATYY | HGNFGDSYVSWFA |
| | | | ADSVKG | Y |
| | Second VL | SEQ ID NO: 28 | SEQ ID NO: 29 | SEQ ID NO: 30 |
| | | GSSTGAVTTSNYA | GTNKRAP | ALWYSNHWV |
| | | N | | |
| SEQ ID NO: 97 | First VL | SEQ ID NO: 1 | SEQ ID NO: 2 | SEQ ID NO: 3 |
| | | SAGSGLYG | GTNKRPS | GSADSSTNAGI |
| | First VH | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | Second VH | SEQ ID NO: 25 | SEQ ID NO: 31 | SEQ ID NO: 32 |
| | | TYAMN | RIRSKYNNYATYY | HGNFGNSYVSWFA |
| | | | ADSVKS | Y |
| | Second VL | SEQ ID NO: 33 | SEQ ID NO: 34 | SEQ ID NO: 35 |
| | | RSSTGAVTTSNYA | GANKRAP | ALWYSNLWV |
| | | N | | |
| SEQ ID NO: 98 | First VL | SEQ ID NO: 13 | SEQ ID NO: 14 | SEQ ID NO: 3 |
| | | SGGSGSYG | GTYKRPS | GSADSSTNAGI |
| | First VH | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | Second VH | SEQ ID NO: 25 | SEQ ID NO: 31 | SEQ ID NO: 32 |
| | | TYAMN | RIRSKYNNYATYY | HGNFGNSYVSWFA |
| | | | ADSVKS | Y |
| | Second VL | SEQ ID NO: 33 | SEQ ID NO: 34 | SEQ ID NO: 35 |
| | | RSSTGAVTTSNYA | GANKRAP | ALWYSNLWV |
| | | N | | |
| SEQ ID NO: 99 | First VL | SEQ ID NO: 1 | SEQ ID NO: 2 | SEQ ID NO: 3 |
| | | SAGSGLYG | GTNKRPS | GSADSSTNAGI |
| | First VH | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | Second VH | SEQ ID NO: 7 | SEQ ID NO: 36 | SEQ ID NO: 9 |
| | | RYTMH | YINPSRGYTNYNQ | YYDDHYCLDY |
| | | | KVKD | |
| | Second VL | SEQ ID NO: 37 | SEQ ID NO: 38 | SEQ ID NO: 39 |
| | | SASSSVSYMN | DTSKLAS | QQWSSNPFT |
| SEQ ID NO: | First VL | SEQ ID NO: 13 | SEQ ID NO: 14 | SEQ ID NO: 3 |
| 100 | | SGGSGSYG | GTYKRPS | GSADSSTNAGI |
| | First VH | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | Second VH | SEQ ID NO: 7 | SEQ ID NO: 36 | SEQ ID NO: 9 |
| | | RYTMH | YINPSRGYTNYNQ | YYDDHYCLDY |
| | | | KVKD | |
| | Second VL | SEQ ID NO: 37 | SEQ ID NO: 38 | SEQ ID NO: 39 |
| | | SASSSVSYMN | DTSKLAS | QQWSSNPFT |
| SEQ ID NO: | First VL | SEQ ID NO: 1 | SEQ ID NO: 2 | SEQ ID NO: 3 |
| 109 | | SAGSGLYG | GTNKRPS | GSADSSTNAGI |
| | First VH | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | Second VH | SEQ ID NO: 40 | SEQ ID NO: 41 | SEQ ID NO: 42 |
| | | GYTMN | LINPYKGVSTYNQ | SGYYGDSDWYFDV |
| | | | KVKG | |
| | Second VL | SEQ ID NO: 43 | SEQ ID NO: 44 | SEQ ID NO: 45 |
| | | RASQDIRNYLN | YTSRLHS | QQGNTLPWT |
| SEQ ID NO: | First VL | SEQ ID NO: 13 | SEQ ID NO: 14 | SEQ ID NO: 3 |
| 110 | | SGGSGSYG | GTYKRPS | GSADSSTNAGI |
| | First VH | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | Second VH | SEQ ID NO: 40 | SEQ ID NO: 41 | SEQ ID NO: 42 |
| | | GYTMN | LINPYKGVSTYNQ | SGYYGDSDWYFDV |
| | | | KVKG | |
| | Second VL | SEQ ID NO: 43 | SEQ ID NO: 44 | SEQ ID NO: 45 |
| | | RASQDIRNYLN | YTSRLHS | QQGNTLPWT |

In some embodiments, disclosed herein is a composition comprising a scFv-Fab Fe antibody specific for claudin 6 and CD3 comprising:
(a) a first heavy chain selected from:
(i) SEQ ID NO: 79,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
(ii) SEQ ID NO: 88,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
(iii) SEQ ID NO: 114,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SMGSGVSWSGYVATSIDV (SEQ ID NO: 47), or a variant thereof,
(b) a second heavy chain selected from:
(i.) SEQ ID NO: 80,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof, or
(ii.) SEQ ID NO: 89,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 26, or a variant thereof, and the heavy chain CDR3 comprises an amino acid sequence of SEQ ID NO: 27, or a variant thereof, or
   a heavy chain variable region comprising heavy chain CDR1, CDR2, and CDR3 sequences, wherein the heavy chain CDR1 comprises an amino acid sequence of SEQ ID NO: 28, or a variant thereof, the heavy chain CDR2 comprises an amino acid sequence of SEQ ID NO: 29, or a variant thereof, and the heavy chain CDR3 comprises an amino acid sequence of SEQ ID NO: 30, or a variant thereof, or
(iii.) SEQ ID NO: 90,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 31, or a variant thereof, and the heavy chain CDR3 comprises an amino acid sequence of SEQ ID NO: 32, or a variant thereof, or
   a heavy chain variable region comprising heavy chain CDR1, CDR2, and CDR3 sequences, wherein the heavy chain CDR1 comprises an amino acid sequence of RSSTGAVTTSNYA (SEQ ID NO: 48), or a variant thereof, the heavy chain CDR2 comprises an amino acid sequence of SEQ ID NO: 34, or a variant thereof, and the heavy chain CDR3 comprises an amino acid sequence of SEQ ID NO: 35, or a variant thereof, or
(iv.) SEQ ID NO: 91,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 19, or a variant thereof, the heavy chain CDR2 comprises an amino acid sequence of SEQ ID NO: 20, or a variant thereof, and the heavy chain CDR3 comprises an amino acid sequence of SEQ ID NO: 21, or a variant thereof, or
   a heavy chain variable region comprising heavy chain CDR1, CDR2, and CDR3 sequences, wherein the heavy chain CDR1 comprises an amino acid sequence is GSSTGAVTSGNYP (SEQ ID NO: 49), or a variant thereof, the heavy chain CDR2 comprises an amino acid sequence of SEQ ID NO: 23, or a variant thereof, and the heavy chain CDR3 comprises an amino acid sequence of SEQ ID NO: 24, or a variant thereof, or
(v.) SEQ ID NO: 92,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 7, or a variant thereof, the heavy chain CDR2 comprises an amino acid sequence of SEQ ID NO: 36, or a variant thereof, and the heavy chain CDR3 comprises an amino acid sequence of SEQ ID NO: 9, or a variant thereof, or
   a heavy chain variable region comprising heavy chain CDR1, CDR2, and CDR3 sequences, wherein the heavy chain CDR1 comprises an amino acid sequence of SEQ ID NO: 37, or a variant thereof, the heavy chain CDR2 comprises an amino acid sequence of SEQ ID NO: 38, or a variant thereof, and the heavy chain CDR3 comprises an amino acid sequence of SEQ ID NO: 39, or a variant thereof, or
(vi.) SEQ ID NO: 111,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 40, or a variant thereof, the heavy chain CDR2 comprises an amino acid sequence of SEQ ID NO: 41, or a variant thereof, and the heavy chain CDR3 comprises an amino acid sequence of SEQ ID NO: 42, or a variant thereof, or
   a heavy chain variable region comprising heavy chain CDR1, CDR2, and CDR3 sequences, wherein the heavy chain CDR1 comprises an amino acid sequence of SEQ ID NO: 43, or a variant thereof, the heavy chain CDR2 comprises an amino acid sequence of SEQ ID NO: 44, or a variant thereof, and the heavy chain CDR3 comprises an amino acid sequence of SEQ ID NO: 45, or a variant thereof; and
(c) a first light chain selected from:
(i) SEQ ID NO: 67,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof, or
(iii) SEQ ID NO: 65,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

In some embodiments, the Fc is from IgG.

In some embodiments, the IgG is human IgG. In some embodiments, the human IgG is selected from IgG1, IgG2, IgG3, and IgG4.

In some embodiments, the antibody is formed through a knob-in-hole interaction in the Fc region. In some embodiments, the human IgG Fc comprises one or mutations to promote knob-in-hole interaction. In some embodiments, the mutations are selected from (i) T366Y or T366W, and (ii) Y407T, Y407A, or Y407V. In some embodiments, the mutations are selected from:
(a) T366Y and Y407T or T366Y/F405A and T394W/Y407T in human IgG1, or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

In some embodiments, the human IgG Fc comprises one or mutations to reduce or eliminate the effector function of the Fc domains. In some embodiments, the mutations are L234A and L235A (LALA) substitutions in human IgG1.

In some embodiments, the human IgG Fc comprises one or mutations to stabilize a hinge region in the Fc domain. In some embodiments, the mutation is S228P.

In some embodiments, the scFv-Fab antibody specific for claudin 6 and CD3 comprise the CDR sequences as shown in Table 2 below:

TABLE 2
Exemplary scFv-Fab sequences

| Domain | SEQ ID NO | CDR1 | CDR2 | CDR3 |
|---|---|---|---|---|
| First Heavy | SEQ ID NO: 79 | SEQ ID NO: 4 | SEQ ID NO: 5 | SEQ ID NO: 6 |
| Chain | | SYAMN | GISSSGRYTGYAD | SVGSGVSWSGYVA |
| | | | SVKG | TSLDA |
| | SEQ ID NO: 88 | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | SEQ ID NO: 114 | SEQ ID NO: 4 | SEQ ID NO: 5 | SEQ ID NO: 47 |
| | | SYAMN | GISSSGRYTGYAD | SMGSGVSWSGYVA |
| | | | SVKG | TSIDV |
| ScFv | SEQ ID NO: 80 | (HCDR1) | (HCDR2) | (HCDR3) |
| | | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | SEQ ID NO: 81 | (HCDR1) | (HCDR2) | (HCDR3) |
| | | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | SEQ ID NO: 89 | (HCDR1) | (HCDR2) | (HCDR3) |
| | | SEQ ID NO: 25 | SEQ ID NO: 26 | SEQ ID NO: 27 |
| | | TYAMN | RIRSKYNNYATYY | HGNFGDSYVSWFA |
| | | | ADSVKG | Y |
| | | (LCDR1) | (LCDR2) | (LCDR3) |
| | | SEQ ID NO: 28 | SEQ ID NO: 29 | SEQ ID NO: 30 |
| | | GSSTGAVTTSNYA | GTNKRAP | ALWYSNHWV |
| | | N | | |
| | SEQ ID NO: 90 | (HCDR1) | (HCDR2) | (HCDR3) |
| | | SEQ ID NO: 25 | SEQ ID NO: 31 | SEQ ID NO: 32 |
| | | TYAMN | RIRSKYNNYATYY | HGNFGNSYVSWFA |
| | | | ADSVKS | Y |
| | | (LCDR1) | (LCDR2) | (LCDR3) |
| | | SEQ ID NO: 48 | SEQ ID NO: 34 | SEQ ID NO: 35 |
| | | RSSTGAVTTSNYA | GANKRAP | ALWYSNLWV |
| | SEQ ID NO: 91 | (HCDR1) | (HCDR2) | (HCDR3) |
| | | SEQ ID NO: 19 | SEQ ID NO: 20 | SEQ ID NO: 21 |
| | | KYAMN | RIRSKYNNYATYY | HGNFGNSYISYWA |
| | | | ADSVKD | Y |
| | | (LCDR1) | (LCDR2) | (LCDR3) |
| | | SEQ ID NO: 49 | SEQ ID NO: 23 | SEQ ID NO: 24 |
| | | GSSTGAVTSGNYP | GTKFLAP | VLWYSNRWV |
| | SEQ ID NO: 92 | (HCDR1) | (HCDR2) | (HCDR3) |
| | | SEQ ID NO: 7 | SEQ ID NO: 36 | SEQ ID NO: 9 |
| | | RYTMH | YINPSRGYTNYNQ | YYDDHYCLDY |
| | | | KVKD | |
| | | (LCDR1) | (LCDR2) | (LCDR3) |
| | | SEQ ID NO: 37 | SEQ ID NO: 38 | SEQ ID NO: 39 |
| | | SASSSVSYMN | DTSKLAS | QQWSSNPFT |
| | SEQ ID NO: 111 | (HCDR1) | (HCDR2) | (HCDR3) |
| | | SEQ ID NO: 40 | SEQ ID NO: 41 | SEQ ID NO: 42 |
| | | GYTMN | LINPYKGVSTYNQ | SGYYGDSDWYFDV |
| | | | KVKG | |
| | | (LCDR1) | (LCDR2) | (LCDR3) |
| | | SEQ ID NO: 43 | SEQ ID NO: 44 | SEQ ID NO: 45 |
| | | RASQDIRNYLN | YTSRLHS | QQGNTLPWT |
| First Light | SEQ ID NO: 65 | SEQ ID NO: 1 | SEQ ID NO: 2 | SEQ ID NO: 3 |
| Chain | | SAGSGLYG | GTNKRPS | GSADSSTNAGI |
| | SEQ ID NO: 66 | SEQ ID NO: 13 | SEQ ID NO: 14 | SEQ ID NO: 3 |
| | | SGGSGSYG | GTYKRPS | GSADSSTNAGI |
| | SEQ ID NO: 67 | SEQ ID NO: 1 | SEQ ID NO: 2 | SEQ ID NO: 3 |
| | | SAGSGLYG | GTNKRPS | GSADSSTNAGI |

In some embodiments, the scFv-Fab antibody specific for claudin 6 and CD3 comprise the CDR sequences as shown in Table 3 below:

TABLE 3
Exemplary scFv-Fab sequences

| Domain | SEQ ID NO | CDR1 | CDR2 | CDR3 |
|---|---|---|---|---|
| First Heavy | SEQ ID NO: 79 | SEQ ID NO: 4 | SEQ ID NO: 5 | SEQ ID NO: 6 |
| Chain | | SYAMN | GISSSGRYTGYAD | SVGSGVSWSGYVA |
| | | | SVKG | TSLDA |
| | SEQ ID NO: 88 | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | SEQ ID NO : | SEQ ID NO: 4 | SEQ ID NO: 5 | SEQ ID NO: 47 |
| | 114 | SYAMN | GISSSGRYTGYAD | SMGSGVSWSGYVA |
| | | | SVKG | TSIDV |
| Second Heavy | SEQ ID NO: 117 | (VH1HCDR1) | (VH1HCDR2) | (VH1HCDR3) |
| Chain | | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | | (VH2HCDR1) | (VH2HCDR2) | (VH2HCDR3) |
| | | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | SEQ ID NO: 118 | (VH1HCDR1) | (VH1HCDR2) | (VH1HCDR3) |
| | | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | | (VH2HCDR1) | (VH2HCDR2) | (VH2HCDR3) |
| | | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | SEQ ID NO: 119 | (VH1HCDR1) | (VH1HCDR2) | (VH1HCDR3) |
| | | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | | (VH2HCDR1) | (VH2HCDR2) | (VH2HCDR3) |
| | | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | SEQ ID NO: 120 | (VH1HCDR1) | (VH1HCDR2) | (VH1HCDR3) |
| | | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | | (VH2HCDR1) | (VH2HCDR2) | (VH2HCDR3) |
| | | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | SEQ ID NO: 121 | SEQ ID NO: 25 | SEQ ID NO: 26 | SEQ ID NO: 27 |
| | | TYAMN | RIRSKYNNYATYY | HGNFGDSYVSWFA |
| | | | ADSVKG | Y |
| | SEQ ID NO: 123 | SEQ ID NO: 25 | SEQ ID NO: 31 | SEQ ID NO: 32 |
| | | TYAMN | RIRSKYNNYATYY | HGNFGNSYVSWFA |
| | | | ADSVKS | Y |
| | SEQ ID NO: 125 | SEQ ID NO: 19 | SEQ ID NO: 20 | SEQ ID NO: 21 |
| | | KYAMN | RIRSKYNNYATYY | HGNFGNSYISYWA |
| | | | ADSVKD | Y |
| | SEQ ID NO: 127 | SEQ ID NO: 7 | SEQ ID NO: 36 | SEQ ID NO: 9 |
| | | RYTMH | YINPSRGYTNYNQ | YYDDHYCLDY |
| | | | KVKD | |
| First Light | SEQ ID NO: 65 | SEQ ID NO: 1 | SEQ ID NO: 2 | SEQ ID NO: 3 |
| Chain | | SAGSGLYG | GTNKRPS | GSADSSTNAGI |
| | SEQ ID NO: 66 | SEQ ID NO: 13 | SEQ ID NO: 14 | SEQ ID NO: 3 |
| | | SGGSGSYG | GTYKRPS | GSADSSTNAGI |
| | SEQ ID NO: 67 | SEQ ID NO: 1 | SEQ ID NO: 2 | SEQ ID NO: 3 |
| | | SAGSGLYG | GTNKRPS | GSADSSTNAGI |
| Second Light | SEQ ID NO: 122 | SEQ ID NO: 28 | SEQ ID NO: 29 | SEQ ID NO: 30 |
| Chain | | GSSTGAVTTSNYA | GTNKRAP | ALWYSNHWV |
| | | N | | |
| | SEQ ID NO: 124 | SEQ ID NO: 33 | SEQ ID NO: 34 | SEQ ID NO: 35 |
| | | RSSTGAVTTSNYA | GANKRAP | ALWYSNLWV |
| | | N | | |
| | SEQ ID NO: 126 | SEQ ID NO: 22 | SEQ ID NO: 23 | SEQ ID NO: 24 |
| | | GSSTGAVTSGNYP | GTKFLAP | VLWYSNRWV |
| | | N | | |
| | SEQ ID NO: 128 | SEQ ID NO: 37 | SEQ ID NO: 38 | SEQ ID NO: 39 |
| | | SASSSVSYMN | DTSKLAS | QQWSSNPFT |
| | SEQ ID NO: 130 | SEQ ID NO: 43 | SEQ ID NO: 44 | SEQ ID NO: 45 |
| | | RASQDIRNYLN | YTSRLHS | QQGNTLPWT |

In some embodiments, the scFv-Fab antibody specific for claudin 6 and CD3 comprise the CDR sequences as shown in Table 4 below:

TABLE 4
Exemplary scFv-Fab antibodies

| Antibody | Domain | CDR1 | CDR2 | CDR3 |
|---|---|---|---|---|
| IMC-16-3 | First Heavy | SEQ ID NO: 4 | SEQ ID NO: 5 | SEQ ID NO: 6 |
| | Chain-SEQ | SYAMN | GISSSGRYTGYAD | SVGSGVSWSGYVA |
| | ID NO: 79 | | SVKG | TSLDA |
| | SCFV-SEQ ID | (HCDR1) | (HCDR2) | (HCDR3) |
| | NO: 89 | SEQ ID NO: 25 | SEQ ID NO: 26 | SEQ ID NO: 27 |
| | | TYAMN | RIRSKYNNYATYY | HGNFGDSYVSWFA |
| | | | ADSVKG | Y |
| | | (LCDR1) | (LCDR2) | (LCDR3) |
| | | SEQ ID NO: 28 | SEQ ID NO: 29 | SEQ ID NO: 30 |
| | | GSSTGAVTTSNYA | GTNKRAP | ALWYSNHWV |
| | | N | | |
| | First Light | SEQ ID NO: 1 | SEQ ID NO: 2 | SEQ ID NO: 3 |
| | Chain-SEQ | SAGSGLYG | GTNKRPS | GSADSSTNAGI |
| | ID NO: 67 | | | |
| IMC-16-7 | First Heavy | SEQ ID NO: 4 | SEQ ID NO: 5 | SEQ ID NO: 6 |
| | Chain-SEQ | SYAMN | GISSSGRYTGYAD | SVGSGVSWSGYVA |
| | ID NO: 79 | | SVKG | TSLDA |
| | SCFV-SEQ ID | (HCDR1) | (HCDR2) | (HCDR3) |
| | NO: 91 | SEQ ID NO: 19 | SEQ ID NO: 20 | SEQ ID NO: 21 |
| | | KYAMN | RIRSKYNNYATYY | HGNFGNSYISYWA |
| | | | ADSVKD | Y |
| | | (LCDR1) | (LCDR2) | (LCDR3) |
| | | SEQ ID NO: 49 | SEQ ID NO: 23 | SEQ ID NO: 24 |
| | | GSSTGAVTSGNYP | GTKFLAP | VLWYSNRWV |
| | First Light | SEQ ID NO: 1 | SEQ ID NO: 2 | SEQ ID NO: 3 |
| | Chain-SEQ | SAGSGLYG | GTNKRPS | GSADSSTNAGI |
| | ID NO: 67 | | | |
| IMC-16-13 | First Heavy | SEQ ID NO: 4 | SEQ ID NO: 5 | SEQ ID NO: 47 |
| | Chain-SEQ | SYAMN | GISSSGRYTGYAD | SMGSGVSWSGYVA |
| | ID NO: 114 | | SVKG | TSIDV |
| | SCFV-SEQ ID | (HCDR1) | (HCDR2) | (HCDR3) |
| | NO: 80 | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | First Light | SEQ ID NO: 1 | SEQ ID NO: 2 | SEQ ID NO: 3 |
| | Chain SEQ | SAGSGLYG | GTNKRPS | GSADSSTNAGI |
| | ID NO: 65 | | | |
| IMC-16-15 | First Heavy | SEQ ID NO: 4 | SEQ ID NO: 5 | SEQ ID NO: 47 |
| | Chain-SEQ | SYAMN | GISSSGRYTGYAD | SMGSGVSWSGYVA |
| | ID NO: 114 | | SVKG | TSIDV |
| | SCFV-SEQ ID | (HCDR1) | (HCDR2) | (HCDR3) |
| | NO: 89 | SEQ ID NO: 25 | SEQ ID NO: 26 | SEQ ID NO: 27 |
| | | TYAMN | RIRSKYNNYATYY | HGNFGDSYVSWFA |
| | | | ADSVKG | Y |
| | | (LCDR1) | (LCDR2) | (LCDR3) |
| | | SEQ ID NO: 28 | SEQ ID NO: 29 | SEQ ID NO: 30 |
| | | GSSTGAVTTSNYA | GTNKRAP | ALWYSNHWV |
| | | N | | |
| | First Light | SEQ ID NO: 1 | SEQ ID NO: 2 | SEQ ID NO: 3 |
| | Chain SEQ | SAGSGLYG | GTNKRPS | GSADSSTNAGI |
| | ID NO: 65 | | | |

In some embodiments, disclosed herein is a composition comprising an IgG-(scFV)₂ antibody specific for claudin 6 and CD3 comprising:
(a) a heavy chain selected from:
   (i.) SEQ ID NO: 86,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
   (ii.) SEQ ID NO: 87,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
   (iii.) SEQ ID NO: 101,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 7, or a variant thereof, the heavy chain CDR2 comprises an amino acid sequence of SEQ ID NO: 36, or a variant thereof, and the heavy chain CDR3 comprises an amino acid sequence of SEQ ID NO: 9, or a variant thereof, or
   a heavy chain variable region comprising heavy chain CDR1, CDR2, and CDR3 sequences, wherein the heavy chain CDR1 comprises an amino acid sequence of SEQ ID NO: 37, or a variant thereof, the heavy chain CDR2 comprises an amino acid sequence of SEQ ID NO: 38, or a variant thereof, and the heavy chain CDR3 comprises an amino acid sequence of SEQ ID NO: 39, or a variant thereof,
   (iv.) SEQ ID NO: 102,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 7, or a variant thereof, the heavy chain CDR2 comprises an amino acid sequence of SEQ ID NO: 36, or a variant thereof, and the heavy chain CDR3 comprises an amino acid sequence of SEQ ID NO: 9, or a variant thereof, or
   a heavy chain variable region comprising heavy chain CDR1, CDR2, and CDR3 sequences, wherein the heavy chain CDR1 comprises an amino acid sequence of SEQ ID NO: 37, or a variant thereof, the heavy chain CDR2 comprises an amino acid sequence of SEQ ID NO: 38, or a variant thereof, and the heavy chain CDR3 comprises an amino acid sequence of SEQ ID NO: 39, or a variant thereof,
   (v.) SEQ ID NO: 103,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 26, or a variant thereof, and the heavy chain CDR3 comprises an amino acid sequence of SEQ ID NO: 27, or a variant thereof, or
   a heavy chain variable region comprising heavy chain CDR1, CDR2, and CDR3 sequences, wherein the heavy chain CDR1 comprises an amino acid sequence of SEQ ID NO: 28, or a variant thereof, the heavy chain CDR2 comprises an amino acid sequence of SEQ ID NO: 29, or a variant thereof, and the heavy chain CDR3 comprises an amino acid sequence of SEQ ID NO: 30, or a variant thereof,
   (vi.) SEQ ID NO: 104,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence is of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 26, or a variant thereof, and the heavy chain CDR3 comprises an amino acid sequence of SEQ ID NO: 27, or a variant thereof, or
   a heavy chain variable region comprising heavy chain CDR1, CDR2, and CDR3 sequences, wherein the heavy chain CDR1 comprises an amino acid sequence of SEQ ID NO: 28, or a variant thereof, the heavy chain CDR2 comprises an amino acid sequence of SEQ ID NO: 29, or a variant thereof, and the heavy chain CDR3 comprises an amino acid sequence of SEQ ID NO: 30, or a variant thereof,
   (vii.) SEQ ID NO: 105,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 31, or a variant thereof, and the heavy chain CDR3 comprises an amino acid sequence of SEQ ID NO: 32, or a variant thereof, or
   a heavy chain variable region comprising heavy chain CDR1, CDR2, and CDR3 sequences, wherein the heavy chain CDR1 comprises an amino acid sequence of SEQ ID NO: 33, or a variant thereof, the heavy chain CDR2 comprises an amino acid sequence of SEQ ID NO: 34, or a variant thereof, and the heavy chain CDR3 comprises an amino acid sequence of SEQ ID NO: 35, or a variant thereof,
   (viii.) SEQ ID NO: 106,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 31, or a variant thereof, and the heavy chain CDR3 comprises an amino acid sequence of SEQ ID NO: 32, or a variant thereof, or
   a heavy chain variable region comprising heavy chain CDR1, CDR2, and CDR3 sequences, wherein the heavy chain CDR1 comprises an amino acid sequence of SEQ ID NO: 33, or a variant thereof, the heavy chain CDR2 comprises an amino acid sequence of SEQ ID NO: 34, or a variant thereof, and the heavy chain CDR3 comprises an amino acid sequence of SEQ ID NO: 35, or a variant thereof,
   (ix.) SEQ ID NO: 107,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 19, or a variant thereof, the heavy chain CDR2 comprises an amino acid sequence of SEQ ID NO: 20, or a variant thereof, and the heavy chain CDR3 comprises an amino acid sequence of SEQ ID NO: 21, or a variant thereof, or
   a heavy chain variable region comprising heavy chain CDR1, CDR2, and CDR3 sequences, wherein the heavy chain CDR1 comprises an amino acid sequence of SEQ ID NO: 22, or a variant thereof, the heavy chain CDR2 comprises an amino acid sequence of SEQ ID NO: 23, or a variant thereof, and the heavy chain CDR3 comprises an amino acid sequence of SEQ ID NO: 24, or a variant thereof,
   (x.) SEQ ID NO: 108,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 19, or a variant thereof, the heavy chain CDR2 comprises an amino acid sequence of SEQ ID NO: 20, or a variant thereof, and the heavy chain CDR3 comprises an amino acid sequence of SEQ ID NO: 21, or a variant thereof, or
   a heavy chain variable region comprising heavy chain CDR1, CDR2, and CDR3 sequences, wherein the heavy chain CDR1 comprises an amino acid sequence of SEQ ID NO: 22, or a variant thereof, the heavy chain CDR2 comprises an amino acid sequence of SEQ ID NO: 23, or a variant thereof, and the heavy chain CDR3 comprises an amino acid sequence of SEQ ID NO: 24, or a variant thereof,
   (xi.) SEQ ID NO: 112,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 40, or a variant thereof, the heavy chain CDR2 comprises an amino acid sequence of SEQ ID NO: 41, or a variant thereof, and the heavy chain CDR3 comprises an amino acid sequence of SEQ ID NO: 42, or a variant thereof, or
   a heavy chain variable region comprising heavy chain CDR1, CDR2, and CDR3 sequences, wherein the heavy chain CDR1 comprises an amino acid sequence of SEQ ID NO: 43, or a variant thereof, the heavy chain CDR2 comprises an amino acid sequence of SEQ ID NO: 44, or a variant thereof, and the heavy chain CDR3 comprises an amino acid sequence of SEQ ID NO: 45, or a variant thereof,
   (xii.) SEQ ID NO: 113,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 40, or a variant thereof, the heavy chain CDR2 comprises an amino acid sequence of SEQ ID NO: 41, or a variant thereof, and the heavy chain CDR3 comprises an amino acid sequence of SEQ ID NO: 42, or a variant thereof, or
   a heavy chain variable region comprising heavy chain CDR1, CDR2, and CDR3 sequences, wherein the heavy chain CDR1 comprises an amino acid sequence of SEQ ID NO: 43, or a variant thereof, the heavy chain CDR2 comprises an amino acid sequence of SEQ ID NO: 44, or a variant thereof, and the heavy chain CDR3 comprises an amino acid sequence of SEQ ID NO: 45, or a variant thereof;
   and
(b) a light chain selected from:
   (i) SEQ ID NO: 67,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
   (ii) SEQ ID NO: 66,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
   or
   (iii) SEQ ID NO: 65,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, or
   wherein:
      any of the above sequences in subsections (a) optionally can have a His6 tag (e.g., HHHHHH (SEQ ID NO: 46)) added to the N or C-terminus and
   any of the above sequences optionally have the linker comprising one or more glycines and serines replaced with another peptide linker or functionally equivalent variation thereof.

In some embodiments, the Fc is from IgG. In some embodiments, the IgG is human gG. In some embodiments, the human IgG is selected from IgG1, IgG2, IgG3, and IgG4.

In some embodiments, the human IgG Fc comprises one or mutations to reduce or eliminate the effector function of the Fc domains. In some embodiments, the mutations are L234A and L235A (LALA) substitutions in human IgG1.

In some embodiments, the human IgG Fc comprises one or mutations to stabilize a hinge region in the Fc domain. In some embodiments, the mutation is S228P.

In some embodiments, the IgG-(scFv)₂ antibody specific for claudin 6 and CD3 comprise the CDR sequences as shown in Table 5 below:

| Domain | SEQ ID NO | CDR1 | CDR2 | CDR3 |
|---|---|---|---|---|
| Heavy Chain | SEQ ID NO: 86 | (VH1 HCDR1) | (VH1 HCDR2) | (VH1 HCDR3) |
| and scFv | | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | | (scFv HCDR1) | (scFv HCDR2) | (scFv HCDR3) |
| | | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | | (scFv LCDR1) | (scFv LCDR2) | (scFv LCDR3) |
| | SEQ ID NO: 87 | (VH1 HCDR1) | (VH1 HCDR2) | (VH1 HCDR3) |
| | | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | | (scFv HCDR1) | (scFv HCDR2) | (scFv HCDR3) |
| | | SEQ ID NO: 16 | SEQ ID NO: 17 | SEQ ID NO: 18 |
| | | GYSMG | AIVWSGGNTYYED | KIRPYIFKIAGQY |
| | | | SVKG | DY |
| | | (scFv LCDR1) | (scFv LCDR2) | (scFv LCDR3) |
| | SEQ ID NO: 101 | (VH1 HCDR1) | (VH1 HCDR2) | (VH1 HCDR3) |
| | | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | | (scFv HCDR1) | (scFv HCDR2) | (scFv HCDR3) |
| | | SEQ ID NO: 7 | SEQ ID NO: 36 | SEQ ID NO: 9 |
| | | RYTMH | YINPSRGYTNYNQ | YYDDHYCLDY |
| | | | KVKD | |
| | | (scFv LCDR1) | (scFv LCDR2) | (scFv LCDR3) |
| | | SEQ ID NO: 37 | SEQ ID NO: 38 | SEQ ID NO: 39 |
| | | SASSSVSYMN | DTSKLAS | QQWSSNPFT |
| | SEQ ID NO: 102 | (VH1 HCDR1) | (VH1 HCDR2) | (VH1 HCDR3) |
| | | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | | (SCFv HCDR1) | (scFv HCDR2) | (scFv HCDR3) |
| | | SEQ ID NO: 7 | SEQ ID NO: 36 | SEQ ID NO: 9 |
| | | RYTMH | YINPSRGYTNYNQ | YYDDHYCLDY |
| | | | KVKD | |
| | | (scFv LCDR1) | (scFv LCDR2) | (scFv LCDR3) |
| | | SEQ ID NO: 37 | SEQ ID NO: 38 | SEQ ID NO: 39 |
| | | SASSSVSYMN | DTSKLAS | QQWSSNPFT |
| | | (VH1 HCDR1) | (VH1 HCDR2) | (VH1 HCDR3) |
| | SEQ ID NO: 103 | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | | (scFv HCDR1) | (scFv HCDR2) | (scFv HCDR3) |
| | | SEQ ID NO: 25 | SEQ ID NO: 26 | SEQ ID NO: 27 |
| | | TYAMN | RIRSKYNNYATYY | HGNFGDSYVSWFA |
| | | | ADSVKG | Y |
| | | (scFv LCDR1) | (scFv LCDR2) | (scFv LCDR3) |
| | | SEQ ID NO: 28 | SEQ ID NO: 29 | SEQ ID NO: 30 |
| | | GSSTGAVTTSNYA | GTNKRAP | ALWYSNHWV |
| | | N | | |
| | SEQ ID NO: 104 | (VH1 HCDR1) | (VH1 HCDR2) | (VH1 HCDR3) |
| | | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | | (scFv HCDR1) | (scFv HCDR2) | (scFv HCDR3) |
| | | SEQ ID NO: 25 | SEQ ID NO: 26 | SEQ ID NO: 27 |
| | | TYAMN | RIRSKYNNYATYY | HGNFGDSYVSWFA |
| | | | ADSVKG | Y |
| | | (scFv LCDR1) | (scFv LCDR2) | (scFv LCDR3) |
| | | SEQ ID NO: 28 | SEQ ID NO: 29 | SEQ ID NO: 30 |
| | | GSSTGAVTTSNYA | GTNKRAP | ALWYSNHWV |
| | | N | | |
| | SEQ ID NO: 105 | (VH1 HCDR1) | (VH1 HCDR2) | (VH1 HCDR3) |
| | | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | | (scFv HCDR1) | (scFv HCDR2) | (scFv HCDR3) |
| | | SEQ ID NO: 25 | SEQ ID NO: 31 | SEQ ID NO: 32 |
| | | TYAMN | RIRSKYNNYATYY | HGNFGNSYVSWFA |
| | | | ADSVKS | Y |
| | | (scFv LCDR1) | (scFv LCDR2) | (scFv LCDR3) |
| | | SEQ ID NO: 33 | SEQ ID NO: 34 | SEQ ID NO: 35 |
| | | RSSTGAVTTSNYA | GANKRAP | ALWYSNLWV |
| | | N | | |
| | SEQ ID NO: 106 | (VH1 HCDR1) | (VH1 HCDR2) | (VH1 HCDR3) |
| | | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | | (scFv HCDR1) | (scFv HCDR2) | (scFv HCDR3) |
| | | SEQ ID NO: 25 | SEQ ID NO: 31 | SEQ ID NO: 32 |
| | | TYAMN | RIRSKYNNYATYY | HGNFGNSYVSWFA |
| | | | ADSVKS | Y |
| | | (scFv LCDR1) | (scFv LCDR2) | (scFv LCDR3) |
| | | SEQ ID NO: 33 | SEQ ID NO: 34 | SEQ ID NO: 35 |
| | | RSSTGAVTTSNYA | GANKRAP | ALWYSNLWV |
| | | N | | |
| | SEQ ID NO: 107 | (VH1 HCDR1) | (VH1 HCDR2) | (VH1 HCDR3) |
| | | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | | (scFv HCDR1) | (scFv HCDR2) | (scFv HCDR3) |
| | | SEQ ID NO: 19 | SEQ ID NO: 20 | SEQ ID NO: 21 |
| | | KYAMN | RIRSKYNNYATYY | HGNFGNSYISYWA |
| | | | ADSVKD | Y |
| | | (SCFV LCDR1) | (scFv LCDR2) | (scFv LCDR3) |
| | | SEQ ID NO: 22 | SEQ ID NO: 23 | SEQ ID NO: 24 |
| | | GSSTGAVTSGNYP | GTKFLAP | VLWYSNRWV |
| | | N | | |
| | SEQ ID NO:108 | (VH1 HCDR1) | (VH1 HCDR2) | (VH1 HCDR3) |
| | | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | | (scFv HCDR1) | (scFv HCDR2) | (scFv HCDR3) |
| | | SEQ ID NO: 19 | SEQ ID NO: 20 | SEQ ID NO: 21 |
| | | KYAMN | RIRSKYNNYATYY | HGNFGNSYISYWA |
| | | | ADSVKD | Y |
| | | (scFv LCDR1) | (scFv LCDR2) | (scFv LCDR3) |
| | | SEQ ID NO: 22 | SEQ ID NO: 23 | SEQ ID NO: 24 |
| | | GSSTGAVTSGNYP | GTKFLAP | VLWYSNRWV |
| | | N | | |
| | SEQ ID NO: 112 | (VH1 HCDR1) | (VH1 HCDR2) | (VH1 HCDR3) |
| | | SEQ ID NO: 4 | SEQ ID NO: 5 | SEQ ID NO: 6 |
| | | SYAMN | GISSSGRYTGYAD | SVGSGVSWSGYVA |
| | | | SVKG | TSLDA |
| | | (scFv HCDR1) | (scFv HCDR2) | (scFv HCDR3) |
| | | SEQ ID NO: 40 | SEQ ID NO: 41 | SEQ ID NO: 42 |
| | | GYTMN | LINPYKGVSTYNQ | SGYYGDSDWYFDV |
| | | | KVKG | |
| | | (scFv LCDR1) | (scFv LCDR2) | (scFv LCDR3) |
| | | SEQ ID NO: 43 | SEQ ID NO: 44 | SEQ ID NO: 45 |
| | | RASQDIRNYLN | YTSRLHS | QQGNTLPWT |
| | SEQ ID NO: 113 | (VH1 HCDR1) | (VH1 HCDR2) | (VH1 HCDR3) |
| | | SEQ ID NO: 4 | SEQ ID NO: 5 | SEQ ID NO: 15 |
| | | SYAMN | GISSSGRYTGYAD | SVGSGVSWSGYVA |
| | | | SVKG | TSLDV |
| | | (scFv HCDR1) | (scFv HCDR2) | (scFv HCDR3) |
| | | SEQ ID NO: 40 | SEQ ID NO: 41 | SEQ ID NO: 42 |
| | | GYTMN | LINPYKGVSTYNQ | SGYYGDSDWYFDV |
| | | | KVKG | |
| | | (scFv LCDR1) | (scFv LCDR2) | (scFv LCDR3) |
| | | SEQ ID NO: 43 | SEQ ID NO: 44 | SEQ ID NO: 45 |
| | | RASQDIRNYLN | YTSRLHS | QQGNTLPWT |
| Light Chain | SEQ ID NO: 65 | SEQ ID NO: 1 | SEQ ID NO: 2 | SEQ ID NO: 3 |
| | | SAGSGLYG | GTNKRPS | GSADSSTNAGI |
| | SEQ ID NO: 66 | SEQ ID NO: 13 | SEQ ID NO: 14 | SEQ ID NO: 3 |
| | | SGGSGSYG | GTYKRPS | GSADSSTNAGI |
| | SEQ ID NO: 67 | SEQ ID NO: 1 | SEQ ID NO: 2 | SEQ ID NO: 3 |
| | | SAGSGLYG | GTNKRPS | GSADSSTNAGI |

In some embodiments, disclosed herein is a composition comprising a scFv-Fab Fe antibody specific for claudin 6 and CD3 comprising:
(a) a first heavy chain selected from:
(i) SEQ ID NO: 79,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(ii) SEQ ID NO: 88,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
   or
(iii) SEQ ID NO: 114,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof,
(b) a second heavy chain selected from:
SEQ ID NO: 81,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
and
(c) a light chain selected from:
(i) SEQ ID NO: 67,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
or
(iii) SEQ ID NO: 65,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

In some embodiments, the Fc is from IgG. In some embodiments, the IgG is human IgG. In some embodiments, the human IgG is selected from IgG1, IgG2, IgG3, and IgG4.

In some embodiments, the antibody is formed through a knob-in-hole interaction in the Fc region. In some embodiments, the human IgG Fc comprises one or mutations to promote knob-in-hole interaction. In some embodiments, the mutations are selected from (i) T366Y or T366W, and (ii) Y407T, Y407A, or Y407V. In some embodiments, mutations are:
(a) T366Y and Y407T or T366Y/F405A and T394W/Y407T in human IgG1 or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

In some embodiments, the human IgG Fc comprises one or mutations to reduce or eliminate the effector function of the Fc domains. In some embodiments, the mutations are L234A and L235A (LALA) substitutions in human IgG1. In some embodiments, the human IgG Fc comprises one or mutations to stabilize a hinge region in the Fc domain. In some embodiments, mutation is S228P.

In some embodiments, disclosed herein is a composition comprising a scFv-Fab Fe antibody specific for claudin 6 and CD3 comprising:
(a) a first heavy chain selected from:
(i) SEQ ID NO: 79,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(ii) SEQ ID NO: 88,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
   or
(iii) SEQ ID NO: 114,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof
(b) a second heavy chain selected from:
(i.) SEQ ID NO: 117,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
(ii.) SEQ ID NO: 119,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
(iii.) SEQ ID NO: 118,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(iv.) SEQ ID NO: 120,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
and
(c) a light chain selected from:
(i) SEQ ID NO: 67,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
or
(iii) SEQ ID NO: 65,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.
For example, the linker of GGGGSGGGGSGGGGS(SEQ ID NO: 50) that is illustrated in various embodiments herein (above and below), can be replaced with a linker of LE, SEQ ID NO: 51, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 131.

In some embodiments, the Fe is from IgG. In some embodiments, the IgG is human IgG. In some embodiments, the human IgG is selected from IgG1, IgG2, IgG3, and IgG4.

In some embodiments, the antibody is formed through a knob-in-hole interaction in the Fc region. In some embodiments, the human IgG Fc comprises one or mutations to promote knob-in-hole interaction. In some embodiments, the mutations are selected from (i) T366Y or T366W, and (ii) Y407T, Y407A, or Y407V. In some embodiments, the mutations are:
(a) T366Y and Y407T or T366Y/F405A and T394W/Y407T in human IgG1 or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

In some embodiments, the human IgG Fc comprises one or mutations to reduce or eliminate the effector function of the Fc domains. In some embodiments, the mutations are L234A and L235A (LALA) substitutions in human IgG1. In some embodiments, the human IgG Fc comprises one or mutations to stabilize a hinge region in the Fc domain. In some embodiments, the mutation is S228P.

In some embodiments, disclosed herein is a composition comprising a scFv-Fab Fc antibody specific for claudin 6 and CD3 comprising:
(a) a first heavy chain selected from:
(i) SEQ ID NO: 79,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(ii) SEQ ID NO: 88,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
   or
(iii) SEQ ID NO: 114,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof,
(b) a second heavy chain selected from:
(i) SEQ ID NO: 121,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 26, or a variant thereof, and the heavy chain CDR3 comprises an amino acid sequence of SEQ ID NO: 27, or a variant thereof,
(ii) SEQ ID NO: 123,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 31, or a variant thereof, and the heavy chain CDR3 comprises an amino acid sequence of SEQ ID NO: 32, or a variant thereof,
(iii) SEQ ID NO: 125,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 19, or a variant thereof, the heavy chain CDR2 comprises an amino acid sequence of SEQ ID NO: 20, or a variant thereof, and the heavy chain CDR3 comprises an amino acid sequence of SEQ ID NO: 21, or a variant thereof,
(iv) SEQ ID NO: 127,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 7, or a variant thereof, the heavy chain CDR2 comprises an amino acid sequence of SEQ ID NO: 36, or a variant thereof, and the heavy chain CDR3 comprises an amino acid sequence of SEQ ID NO: 9, or a variant thereof,
(v) SEQ ID NO: 129,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 40, or a variant thereof, the heavy chain CDR2 comprises an amino acid sequence of SEQ ID NO: 41, or a variant thereof, and the heavy chain CDR3 comprises an amino acid sequence of SEQ ID NO: 42, or a variant thereof, and
(c) a first light chain selected from:
(i) SEQ ID NO: 67,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
or
(iii) SEQ ID NO: 65,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, and
(d) a second light chain selected from:
(i) SEQ ID NO: 122,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 28, or a variant thereof, the light chain CDR2 comprises an amino acid sequence of SEQ ID NO: 29, or a variant thereof, and the light chain CDR3 comprises an amino acid sequence of SEQ ID NO: 30, or a variant thereof, or
(ii) SEQ ID NO: 124,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 33, or a variant thereof, the light chain CDR2 comprises an amino acid sequence of SEQ ID NO: 34, or a variant thereof, and the light chain CDR3 comprises an amino acid sequence of SEQ ID NO: 35, or a variant thereof, or
(iii) SEQ ID NO: 126,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 22, or a variant thereof, the light chain CDR2 comprises an amino acid sequence of SEQ ID NO: 23, or a variant thereof, and the light chain CDR3 comprises an amino acid sequence of SEQ ID NO: 24, or a variant thereof, or
(iv) SEQ ID NO: 128,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 37, or a variant thereof, the light chain CDR2 comprises an amino acid sequence of SEQ ID NO: 38, or a variant thereof, and the light chain CDR3 comprises an amino acid sequence of SEQ ID NO: 39, or a variant thereof; or
(v) SEQ ID NO: 130
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 43, or a variant thereof, the light chain CDR2 comprises an amino acid sequence of SEQ ID NO: 44, or a variant thereof, and the light chain CDR3 comprises an amino acid sequence of SEQ ID NO: 45, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

In some embodiments, disclosed herein is a bispecific antibody, wherein the bispecific antibody comprises three polypeptides (e.g., a first polypeptide, a second polypeptide, and a third polypeptide) that form a first antigen binding domain that binds to CLDN6, and a second antigen binding domain that binds to CD3. In some embodiments, the first polypeptide comprises a first light chain comprising a first variable light chain region (first V_{L}), wherein the first variable light chain region comprises a CDR1 sequence comprising the amino acid sequence of SEQ ID NO: 1, a CDR2 sequence comprising the amino acid sequence of SEQ ID NO: 2, and a CDR3 sequence comprising the amino acid sequence of SEQ ID NO: 3. In some embodiments, the second polypeptide comprises a first heavy chain comprising a first variable region heavy chain region (first VH), wherein the first variable heavy chain region comprises a CDR1 sequence comprising the amino acid sequence of SEQ ID NO: 4, a CDR2 sequence comprising the amino acid sequence of SEQ ID NO: 5, and a CDR3 sequence comprising the amino acid sequence of SEQ ID NO: 6. In some embodiments, the third polypeptide comprises a second heavy chain and a second a light chain, wherein the second heavy chain comprises a second variable heavy chain region (second VH) comprising a CDR1 sequence comprising the amino acid sequence of SEQ ID NO: 25, a CDR2 sequence comprising the amino acid sequence of SEQ ID NO: 26, and a CDR3 sequence comprising the amino acid sequence of SEQ ID NO: 27; wherein the second light chain comprises a second variable light chain region (second V_{L}) comprising a CDR1 sequence comprising the amino sequence of SEQ ID NO: 28, a CDR2 sequence comprising the amino acid sequence of SEQ ID NO: 29, and a CDR3 sequence comprising the amino acid sequence of SEQ ID NO: 30. In some embodiments, the second heavy chain and the second light chain are linked by a peptide linker. In some embodiments, the peptide linker is as described herein. In some embodiments, the peptide linker comprises the amino acid sequence of SEQ ID NO: 53. In some embodiments, the peptide linker comprising one or more glycines and serines is replaced with another peptide linker or functionally equivalent variation thereof. In some embodiments, the first V_{L} and the first VH interact to form the antigen binding domain that binds to CLDN6. In some embodiments, the second V_{L} and the second VH interact to form the antigen binding domain that binds to CD3.

In some embodiments, the second V_{L} and the second VH are in a scFv format. In some embodiments, the first V_{L} and the first VH are in a Fab format, or a fragment thereof.

In some embodiments, the first variable light chain region of the first polypeptide comprises the amino acid sequence of SEQ ID NO: 68, or an amino acid sequence having at least about 90%, or about 93%, or about 95%, or about 97%, or about 98%, or about 99% identity thereto.

In some embodiments, the first variable heavy chain region of the second polypeptide comprises the amino acid sequence of SEQ ID NO: 69, or an amino acid sequence having at least about 90%, or about 93%, or about 95%, or about 97%, or about 98%, or about 99% identity thereto.

In some embodiments, the second variable heavy chain region of the third polypeptide comprises the amino acid sequence of:

(SEQ ID NO: 70)
EVQLVESGGGLVQPGGSLRLSCAASGFTFSTYAMNWVR
QAPGKGLEWVGRIRSKYNNYATYYADSVKGRFTISRDD
SKNTLYLQMNSLRAEDTAVYYCVRHGNFGDSYVSWFA
YWGQGTLVTVSS,

or an amino acid sequence having at least about 90%, or about 93%, or about 95%, or about 97%, or about 98%, or about 99% identity thereto.

In some embodiments, the second variable light chain region of the third polypeptide comprises the amino acid sequence of:

(SEQ ID NO: 71)
QAVVTQEPSLTVSPGGTVTLTCGSSTGAVTTSNYANWV
QQKPGKSPRGLIGGTNKRAPGVPARFSGSLLGGKAALTI
SGAQPEDEADYYCALWYSNHWVFGGGTKLTVL,

or an amino acid sequence having at least about 90%, or about 93%, or about 95%, or about 97%, or about 98%, or about 99% identity thereto.

In some embodiments, the first polypeptide compromising the light chain comprises the first variable light chain region and a light chain constant domain, which can be referred to as the first light chain constant domain. In some embodiments, the first light chain constant domain comprises the amino acid sequence of:

(SEQ ID NO: 72)
GQPKAAPSVTLFPPSSEELQANKATLVCLISDFYPGAVT
VAWKADSSPVKAGVETTTPSKQSNNKYAASSYLSLTPE
QWKSHRSYSCQVTHEGSTVEKTVAPTECS,

or an amino acid sequence having at least about 90%, or about 93%, or about 95%, or about 97%, or about 98%, or about 99% identity thereto.

In some embodiments, the first polypeptide comprises the amino acid sequence of SEQ ID NO: 67, or an amino acid sequence having at least about 90%, or about 93%, or about 95%, or about 97%, or about 98%, or about 99% identity thereto.

In some embodiments, the second polypeptide compromising the first heavy chain comprises the first variable heavy chain region and a heavy chain constant domain, which can be referred to as the first heavy chain constant domain. In some embodiments, the first heavy chain constant domain comprises the amino acid sequence of:

(SEQ ID NO: 73)
ASTKGPSVFPLAPSSKSTSGGTAALGCLVKDYFPEPVTV
SWNSGALTSGVHTFPAVLQSSGLYSLSSVVTVPSSSLGT
QTYICNVNHKPSNTKVDKKVEPKSCDKTHTCPPCPAPEA
AGGPSVFLFPPKPKDTLMISRTPEVTCVVVDVSHEDPEV
KFNWYVDGVEVHNAKTKPREEQYNSTYRVVSVLTVLH
QDWLNGKEYKCKVSNKALPAPIEKTISKAKGQPREPQV
YTLPPSREEMTKNQVSLWCLVKGFYPSDIAVEWESNGQ
PENNYKTTPPVLDSDGSFFLYSKLTVDKSRWQQGNVFS
CSVMHEALHNHYTQKSLSLSPGK,

or an amino acid sequence having at least about 90%, or about 93%, or about 95%, or about 97%, or about 98%, or about 99% identity thereto.

In some embodiments, the second polypeptide comprises the amino acid sequence of SEQ ID NO: 79, or an amino acid sequence having at least about 90%, or about 93%, or about 95%, or about 97%, or about 98%, or about 99% identity thereto.

In some embodiments, the third polypeptide comprises a constant domain. In some embodiments, the constant domain is linked to the C-terminus of the second variable light chain region. In some embodiments, there is no peptide linker between the C-terminus of the second variable light chain region and the constant domain. In some embodiments, the constant domain present in the third polypeptide comprises the amino acid sequence of:

(SEQ ID NO: 74)
ASPKSSDKTHTCPPCPAPEAAGGPSVFLFPPKPKDTLMIS
RTPEVTCVVVDVSHEDPEVKFNWYVDGVEVHNAKTKP
REEQYNSTYRVVSVLTVLHQDWLNGKEYKCKVSNKAL
PAPIEKTISKAKGQPREPQVYTLPPSREEMTKNQVSLSCA
VKGFYPSDIAVEWESNGQPENNYKTTPPVLDSDGSFFLV
SKLTVDKSRWQQGNVFSCSVMHEALHNHYTQKSLSLSP
GK,

or an amino acid sequence having at least about 90%, or about 93%, or about 95%, or about 97%, or about 98%, or about 99% identity thereto.

In some embodiments, the third polypeptide comprises the amino acid sequence of SEQ ID NO: 89, or an amino acid sequence having at least about 90%, or about 93%, or about 95%, or about 97%, or about 98%, or about 99% identity thereto.

In some embodiments, the first polypeptide comprises the amino acid sequence of SEQ ID NO: 67; the second polypeptide comprises the amino acid sequence of SEQ ID NO: 79; and the third polypeptide comprises the amino acid sequence of SEQ ID NO: 89.

As used herein, the term constant domain refers to the Fc domain. The constant domains exemplified above are optional embodiments and other constant domains can be substituted for the constant domains described herein.

In some embodiments, the Fc is from IgG. In some embodiments, the IgG is human IgG. In some embodiments, the human IgG is selected from IgG1, IgG2, IgG3, and IgG4.

In some embodiments, the antibody is formed through a knob-in-hole interaction in the Fc region. In some embodiments, the human IgG Fc comprises one or mutations to promote knob-in-hole interaction. In some embodiments, the mutations are selected from (i) T366Y or T366W, and (ii) Y407T, Y407A, or Y407V. In some embodiments, the mutations are:
(a) T366Y and Y407T or T366Y/F405A and T394W/Y407T in human IgG1 or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

In some embodiments, the human IgG Fc comprises one or mutations to reduce or eliminate the effector function of the Fc domains. In some embodiments, the mutations are L234A and L235A (LALA) substitutions in human IgG1. In some embodiments, the human IgG Fc comprises one or mutations to stabilize a hinge region in the Fc domain. In some embodiments, the mutation is S228P.

In some embodiments, the composition binds claudin 6 and CD3 contemporaneously.

In some embodiments, the composition binds claudin 6 with an affinity of less than 10 nM and with at least 100 fold greater affinity than claudin 9, claudin 3, and/or claudin 4.

In any of the embodiments disclosed herein, the composition induces cellular cytotoxicity.

In any of the embodiments disclosed herein, the composition induces T cell cytotoxicity.

In any of the embodiments disclosed herein, the composition induces T cell dependent cytotoxicity.

In any of the embodiments disclosed herein, the composition increases the expression and/or the release of one or more cytokines. In any of the embodiments disclosed herein, the composition increases the expression and/or the release of one or more cytokines selected from IL-2, IL-6, IL-10, IFN-7, and TNF-α.

As provided for herein, an antibody can be formed by the heterodimerization of two Fc domains that are linked to antibody variable domains, which can be either be a VH or can be linked to a scFv format antibody. In some embodiments, the antibody polypeptides comprise a first constant domain and a second constant domain. The constant domain can be based on IgG1, IgG2, IgG3, or IgG4. In some embodiments, the constant domain is a human constant domain. As provided for herein, the constant domain is based on human IgG1 constant domain, which is provided for herein. These constant domains can be incorporated into any of the formats of the antibodies provided for herein.

In some embodiments, the first constant domain comprises a T366W mutation and second constant domain comprises T366S, L368A and Y407V mutations. In some embodiments, the first constant domain comprises T366Y and Y407T mutations or T366Y and F405A mutations and the second constant domain comprises T394W and Y407T mutations.

In some embodiments, the first constant domain comprises T366W and D399C mutations and the second constant domain comprises T366S, L368A, K392C, and Y407V mutations. In some embodiments, the first constant domain comprises T366W and K392C mutations and the second constant domain comprises T366S, L368A, D399C and Y407V mutations. In some embodiments, the first constant domain comprises S354C and T366W mutations and the second constant domain comprises Y349C, T366S, L368A and Y407V mutations. In some embodiments, the first constant domain comprises Y349C and T366W mutations and the second constant domain comprises S354C, T366S, L368A and Y407V mutations. In some embodiments, the first constant domain comprises E356C and T366W mutations and the second constant domain comprises Y349C, T366S, L368A and Y407V mutations. In some embodiments, the first constant domain comprises Y349C and T366W mutations and the second constant domain comprises E356C, T366S, L368A and Y407V mutations. In some embodiments, the first constant domain comprises E357C and T366W mutations and the second constant domain comprises Y349C, T366S, L368A and Y407V mutations. In some embodiments, the first constant domain comprises Y349C and T366W mutations and the second constant domain comprises E357C, T366S, L368A and Y407V mutations.

In some embodiments, first and second constant domains each, independently, comprise L234A and L235A (LALA) substitutions, wherein the numbering is according to the EU numbering in human IgG1. In some embodiments, the first and second constant domains each comprise the L234A and L235A (LALA) substitutions.

The constant domains (Fc), such as the first and second constant domains can also comprise other mutations as provided for herein. These mutations can confer increased specificity for Fc receptor types, such as FcgRIIA.

### Pharmaceutical Compositions

In some embodiments, disclosed herein is a pharmaceutical composition comprising an isolated antibody of any one of the preceding embodiments, or a nucleic acid molecule encoding the same. In some embodiments, the composition is an injectable pharmaceutical composition. In some embodiments, the composition is sterile. In some embodiments, the composition is pyrogen free.

In addition, this document also provides pharmaceutical compositions that composition as described herein, in combination with a pharmaceutically acceptable carrier. A “pharmaceutically acceptable carrier” (also referred to as an “excipient” or a “carrier”) is a pharmaceutically acceptable solvent, suspending agent, stabilizing agent, or any other pharmacologically inert vehicle for delivering one or more therapeutic compounds to a subject (e.g., a mammal, such as a human, non-human primate, dog, cat, sheep, pig, horse, cow, mouse, rat, or rabbit), which is nontoxic to the cell or subject being exposed thereto at the dosages and concentrations employed. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with amino acids include, by way of example and not limitation: water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate). Pharmaceutically acceptable carriers also include aqueous pH buffered solutions or liposomes (small vesicles composed of various types of lipids, phospholipids and/or surfactants which are useful for delivery of a drug to a mammal). Further examples of pharmaceutically acceptable carriers include buffers such as phosphate, citrate, and other organic acids, antioxidants such as ascorbic acid, low molecular weight (less than about 10 residues) polypeptides, proteins such as serum albumin, gelatin, or immunoglobulins, hydrophilic polymers such as polyvinylpyrrolidone, amino acids such as glycine, glutamine, asparagine, arginine or lysine, monosaccharides, disaccharides, and other carbohydrates including glucose, mannose or dextrins, chelating agents such as EDTA, sugar alcohols such as mannitol or sorbitol, salt-forming counterions such as sodium, and/or nonionic surfactants such as TWEEN™, polyethylene glycol (PEG), and PLURONICS™ Pharmaceutical compositions can be formulated by mixing one or more active agents with one or more physiologically acceptable carriers, diluents, and/or adjuvants, and optionally other agents that are usually incorporated into formulations to provide improved transfer, delivery, tolerance, and the like. A pharmaceutical composition can be formulated, e.g., in lyophilized formulations, aqueous solutions, dispersions, or solid preparations, such as tablets, dragees or capsules. A multitude of appropriate formulations can be found in the formulary known to all pharmaceutical chemists: Remington's Pharmaceutical Sciences (18th ed, Mack Publishing Company, Easton, PA (1990)), particularly Chapter 87 by Block, Lawrence, therein. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies as described herein, provided that the active agent in the formulation is not inactivated by the formulation and the formulation is physiologically compatible and tolerable with the route of administration. See, also, Baldrick, Regul Toxicol Pharmacol 32:210-218, 2000; Wang, Int J Pharm 203:1-60, 2000; Charman J Pharm Sci 89:967-978, 2000; and Powell et al. PDA J Pharm Sci Technol 52:238-311, 1998), and the citations therein for additional information related to formulations, excipients and carriers well known to pharmaceutical chemists.

Pharmaceutical compositions include, without limitation, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Compositions and formulations can contain sterile aqueous solutions, which also can contain buffers, diluents and other suitable additives (e.g., penetration enhancers, carrier compounds and other pharmaceutically acceptable carriers). Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the polypeptide components within the compositions provided herein. The formulations can be sterilized if desired.

In some embodiments, a composition containing a composition as provided herein can be in the form of a solution or powder with or without a diluent to make an injectable suspension. The composition may contain additional ingredients including, without limitation, pharmaceutically acceptable vehicles, such as saline, water, lactic acid, mannitol, or combinations thereof, for example.

Any appropriate method can be used to administer a composition as described herein to a mammal. Administration can be, for example, parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). In some embodiments, administration can be topical (e.g., transdermal, sublingual, ophthalmic, or intranasal), pulmonary (e.g., by inhalation or insufflation of powders or aerosols), or oral. In addition, a composition containing a composition as described herein can be administered prior to, after, or in lieu of surgical resection of a tumor.

The antibodies, compositions, or pharmaceutical compositions provided for herein may be administered at any appropriate interval to achieve the desired effect in a subject. In some embodiments, the antibodies, compositions, or pharmaceutical compositions, are administered daily, every other day, weekly, biweekly, once every three weeks, or monthly (i.e. once every four weeks). In some embodiments, a method as provided for herein comprises administering an antibody, composition, or pharmaceutical composition to a cell or to a subject in need thereof at an interval of daily, every other day, weekly, biweekly, once every three weeks, or monthly (i.e. once every four weeks).

In some embodiments, disclosed herein is a nucleic acid molecule encoding an antibody or an amino acid sequence of any of the preceding embodiments.

In some embodiments, disclosed herein is a vector comprising the nucleic acid molecule of any of the preceding embodiments.

In some embodiments, disclosed herein is a cell comprising the nucleic acid molecule of any of the preceding embodiments, or the vector of any of the preceding embodiments.

In some embodiments, disclosed herein is a method for modulating and/or targeting claudin 6 and CD3 in a biological cell, comprising contacting the cell with a composition of any of the preceding embodiments.

In some embodiments, disclosed herein is a method for modulating claudin 6 activity in a biological cell comprising contacting a cell expressing claudin 6 with a composition of any of the preceding embodiments.

In some embodiments, disclosed herein is a method for inhibiting the function of claudin 6 in a biological cell comprising contacting a cell expressing claudin 6 with a composition of any of the preceding embodiments.

### Methods of Treatment

In some embodiments, disclosed herein is a method for treating or preventing cancer comprising administering an effective amount of the composition of any of the preceding embodiments to a subject in need thereof.

In some embodiments, disclosed herein is a use of the composition of any of the preceding embodiments for the preparation of a medicament for the treatment of prevention of cancer.

In some embodiments, disclosed herein is a method or use of any one of the preceding embodiments, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; glioma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; malignant rhabdoid tumor; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

As a non-limiting example, the prevention of an onset, the presence, and/or the evaluation of the progression of a cancer in a subject can be assessed according to the Tumor/Nodes/Metastases (TNM) system of classification (International Union Against Cancer, 6th edition, 2002), or the Whitmore-Jewett staging system (American Urological Association). Typically, cancers are staged using a combination of physical examination, blood tests, and medical imaging. If tumor tissue is obtained via biopsy or surgery, examination of the tissue under a microscope can also provide pathologic staging. In some embodiments, the stage or grade of a cancer assists a practitioner in determining the prognosis for the cancer and in selecting the appropriate modulating therapy.

In some embodiments, the prevention of an onset, or progression, of cancer is assessed using the overall stage grouping as a non-limiting example: Stage I cancers are localized to one part of the body, typically in a small area; Stage II cancers are locally advanced and have grown into nearby tissues or lymph nodes, as are Stage III cancers. Whether a cancer is designated as Stage II or Stage III can depend on the specific type of cancer. The specific criteria for Stages II and III can differ according to diagnosis. Stage IV cancers have often metastasized or spread to other organs or throughout the body. The onset or progression of cancer can be assessed using conventional methods available to one of skill in the art, such as a physical exam, blood tests, and imaging scans (e.g., X-rays, MRI, CT scans, ultrasound etc.).

As disclosed herein, administering, or administering a treatment/therapy, refers to a treatment/therapy from which a subject receives a beneficial effect, such as the reduction, decrease, attenuation, diminishment, stabilization, remission, suppression, inhibition or arrest of the development or progression of cancer, or a symptom thereof.

In some embodiments, the treatment/therapy that a subject receives, or the prevention in the onset of cancer results in at least one or more of the following effects: (1) the reduction or amelioration of the severity of cancer and/or a genetic disease or disorder, and/or a symptom associated therewith; (2) the reduction in the duration of a symptom associated with cancer and/or a genetic disease or disorder; (3) the prevention in the recurrence of a symptom associated with cancer and/or a genetic disease or disorder; (4) the regression of cancer and/or a genetic disease or disorder, and/or a symptom associated therewith; (5) the reduction in hospitalization of a subject; (6) the reduction in hospitalization length; (7) the increase in the survival of a subject; (8) the inhibition of the progression of cancer and/or a genetic disease or disorder and/or a symptom associated therewith; (9) the enhancement or improvement the therapeutic effect of another therapy; (10) a reduction or elimination in the cancer cell population, and/or a cell population associated with a genetic disease or disorder; (11) a reduction in the growth of a tumor or neoplasm; (12) a decrease in tumor size; (13) a reduction in the formation of a tumor; (14) eradication, removal, or control of primary, regional and/or metastatic cancer; (15) a decrease in the number or size of metastases; (16) a reduction in mortality; (17) an increase in cancer-free survival rate of a subject; (18) an increase in relapse-free survival; (19) an increase in the number of subjects in remission; (20) a decrease in hospitalization rate; (21) the size of the tumor is maintained and does not increase in size or increases the size of the tumor by less 5% or 10% after administration of a therapy as measured by conventional methods available to one of skill in the art, e.g., X-rays, MRI, CAT scan, ultrasound etc.; (22) the prevention of the development or onset of cancer and/or a genetic disease or disorder, and/or a symptom associated therewith; (23) an increase in the length of remission for a subject; (24) the reduction in the number of symptoms associated with cancer and/or a genetic disease or disorder; (25) an increase in symptom-free survival of a cancer subject and/or a subject associated with a genetic disease or disorder; and/or (26) limitation of or reduction in metastasis. In some embodiments, the treatment/therapy that a subject receives does not cure cancer, but prevents the progression or worsening of the disease. In certain embodiments, the treatment/therapy that a subject receives does not prevent the onset/development of cancer, but may prevent the onset of cancer symptoms.

In some embodiments, the subject that is treated does not develop cytokine release syndrome (CRS), or does not develop significant CRS-associated clinical symptoms or toxicity. These symptoms include, but are not limited to fever, chills, fatigue, weakness, loss of appetite, nausea, vomiting, diarrhea, headache, joint or muscle aches, skin rash, low blood pressure, increased heart rate, irregular heartbeat, tachycardia, decreased heart function, swelling, buildup of fluids (edema), confusion, dizziness, seizures, hallucinations, decreased coordination, problems talking or swallowing, shaking, problems controlling movements, cough, shortness of breath, tachypnoea, decreased lung function, reduced oxygen levels, decreased kidney or liver function, increased cytokine levels in the blood, change in electrolytes, change in blood clotting. In some embodiments, there is no increase or no significant increase in the blood levels of interleukin-6 (IL-6), interleukin-10 (IL-10), interferon (IFN)-γ, monocyte chemoattractant protein 1 (MCP-1) granulocyte-macrophage colony-stimulating factor (GM-CSF), tumor necrosis factor (TNF), IL-1, IL-2, IL-2-receptor-α, or IL-8 after administration of an antibody as provided for herein.

In some embodiments, “preventing” an onset or progression of cancer in a subject in need thereof, is inhibiting or blocking the cancer or disorder. In some embodiments, the methods disclosed herein prevent, or inhibit, the cancer or disorder at any amount or level. In some embodiments, the methods disclosed herein prevent or inhibit the cancer or genetic disease or disorder by at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition, or at least or about a 100% inhibition).

In some embodiments, disclosed herein is an isolated antibody comprising one or more of the sequences disclosed herein.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

### ENUMERATED EMBODIMENTS

In some embodiments, the following embodiments are provided:

1. A composition comprising a tandem single-chain variable fragment (scFv) specific for claudin 6 and CD3 comprising one or more or:
I.
(a) an amino acid sequence of SEQ ID NO. 82, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4 or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of SEQ ID NO: 7, or a variant thereof, the HCDR2 comprises an amino acid sequence of SEQ ID NO: 8, or a variant thereof, and the HCDR3 comprises an amino acid sequence of SEQ ID NO: 9, or a variant thereof, and
   (iv) a VL comprising LCDR1, LCDR2, and LCDR3 sequences, wherein the LCDR1 comprises an amino acid sequence of SEQ ID NO: 10, or a variant thereof, the LCDR2 comprises an amino acid sequence of SEQ ID NO: 11, or a variant thereof, and the LCDR3 comprises an amino acid sequence of SEQ ID NO: 12, or a variant thereof,
II.
(a) an amino acid sequence of SEQ ID NO. 83, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 7, or a variant thereof, the HCDR2 comprises an amino acid sequence of SEQ ID NO: 8, or a variant thereof, and the HCDR3 comprises an amino acid sequence of SEQ ID NO: 9, or a variant thereof, and
   (iv) a VL comprising LCDR1, LCDR2, and LCDR3 sequences, wherein the LCDR1 comprises an amino acid sequence of SEQ ID NO: 10, or a variant thereof, the LCDR2 comprises an amino acid sequence of SEQ ID NO: 11, or a variant thereof, and the LCDR3 comprises an amino acid sequence of SEQ ID NO: 12, or a variant thereof,
III.
(a) an amino acid sequence of SEQ ID NO. 84, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof, and
   (iii) a VH comprising HCDR1, HCDR2, and HCDR3 sequences, wherein the HCDR1 comprises an amino acid sequence of SEQ ID NO: 16, or a variant thereof, the HCDR2 comprises an amino acid sequence of SEQ ID NO: 17, or a variant thereof, and the HCDR3 comprises an amino acid sequence of SEQ ID NO: 18, or a variant thereof, IV.
(a) an amino acid sequence of SEQ ID NO: 85, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof, and
   (iii) a VH comprising HCDR1, HCDR2, and HCDR3 sequences, wherein the HCDR1 comprises an amino acid sequence of SEQ ID NO: 16, or a variant thereof, the HCDR2 comprises an amino acid sequence of SEQ ID NO: 17, or a variant thereof, and the HCDR3 comprises an amino acid sequence of SEQ ID NO: 18, or a variant thereof,
V.
(a) an amino acid sequence of SEQ ID NO. 93, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of SEQ ID NO: 19, or a variant thereof, the HCDR2 comprises an amino acid sequence of SEQ ID NO: 20, or a variant thereof, and the HCDR3 comprises an amino acid sequence of SEQ ID NO: 21, or a variant thereof, and
   (iv) a VL comprising LCDR1, LCDR2, and LCDR3 sequences, wherein the LCDR1 comprises an amino acid sequence of SEQ ID NO: 22, or a variant thereof, the LCDR2 comprises an amino acid sequence of SEQ ID NO: 23, or a variant thereof, and the LCDR3 comprises an amino acid sequence of SEQ ID NO: 24, or a variant thereof,
VI.
(a) an amino acid sequence of SEQ ID NO. 94, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 19, or a variant thereof, the HCDR2 comprises an amino acid sequence of SEQ ID NO: 20, or a variant thereof, and the HCDR3 comprises an amino acid sequence of SEQ ID NO: 21, or a variant thereof, and
   (iv) a VL comprising LCDR1, LCDR2, and LCDR3 sequences, wherein the LCDR1 comprises an amino acid sequence of SEQ ID NO: 22, or a variant thereof, the LCDR2 comprises an amino acid sequence of SEQ ID NO: 23, or a variant thereof, and the LCDR3 comprises an amino acid sequence of SEQ ID NO: 24, or a variant thereof,
VII.
(a) an amino acid sequence of SEQ ID NO. 95, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence is of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of SEQ ID NO: 25, or a variant thereof, the HCDR2 comprises an amino acid sequence of SEQ ID NO: 26, or a variant thereof, and the HCDR3 comprises an amino acid sequence of SEQ ID NO: 27, or a variant thereof, and
   (iv) a VL comprising LCDR1, LCDR2, and LCDR3 sequences, wherein the LCDR1 comprises an amino acid sequence of SEQ ID NO: 28, or a variant thereof, the LCDR2 comprises an amino acid sequence of SEQ ID NO: 29, or a variant thereof, and the LCDR3 comprises an amino acid sequence of SEQ ID NO: 30, or a variant thereof,
VIII.
(a) an amino acid sequence of SEQ ID NO. 96, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 25, or a variant thereof, the HCDR2 comprises an amino acid sequence of SEQ ID NO: 26, or a variant thereof, and the HCDR3 comprises an amino acid sequence of SEQ ID NO: 27, or a variant thereof, and
   (iv) a VL comprising LCDR1, LCDR2, and LCDR3 sequences, wherein the LCDR1 comprises an amino acid sequence of SEQ ID NO: 28, or a variant thereof, the LCDR2 comprises an amino acid sequence of SEQ ID NO: 29, or a variant thereof, and the LCDR3 comprises an amino acid sequence of SEQ ID NO: 30, or a variant thereof,
IX.
(a) an amino acid sequence of SEQ ID NO. 97, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of SEQ ID NO: 25, or a variant thereof, the HCDR2 comprises an amino acid sequence of SEQ ID NO: 31, or a variant thereof, and the HCDR3 comprises an amino acid sequence of SEQ ID NO: 32, or a variant thereof, and
   (iv) a VL comprising LCDR1, LCDR2, and LCDR3 sequences, wherein the LCDR1 comprises an amino acid sequence of SEQ ID NO: 33, or a variant thereof, the LCDR2 comprises an amino acid sequence of SEQ ID NO: 34, or a variant thereof, and the LCDR3 comprises an amino acid sequence of SEQ ID NO: 35, or a variant thereof,
X.
(a) an amino acid sequence of SEQ ID NO. 98, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 25, or a variant thereof, the HCDR2 comprises an amino acid sequence of SEQ ID NO: 31, or a variant thereof, and the HCDR3 comprises an amino acid sequence of SEQ ID NO: 32, or a variant thereof, and
   (iv) a VL comprising LCDR1, LCDR2, and LCDR3 sequences, wherein the LCDR1 comprises an amino acid sequence of SEQ ID NO: 33, or a variant thereof, the LCDR2 comprises an amino acid sequence of SEQ ID NO: 34, or a variant thereof, and the LCDR3 comprises an amino acid sequence of SEQ ID NO: 35, or a variant thereof,
XI.
(a) an amino acid sequence of SEQ ID NO. 99, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of SEQ ID NO: 7, or a variant thereof, the HCDR2 comprises an amino acid sequence of SEQ ID NO: 36, or a variant thereof, and the HCDR3 comprises an amino acid sequence of SEQ ID NO: 9, or a variant thereof, and
   (iv) a VL comprising LCDR1, LCDR2, and LCDR3 sequences, wherein the LCDR1 comprises an amino acid sequence of SEQ ID NO: 37, or a variant thereof, the LCDR2 comprises an amino acid sequence of SEQ ID NO: 38, or a variant thereof, and the LCDR3 comprises an amino acid sequence of SEQ ID NO: 39, or a variant thereof,
XII.
(a) an amino acid sequence of SEQ ID NO. 100, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 7, or a variant thereof, the HCDR2 comprises an amino acid sequence of ID NO: 36, or a variant thereof, and the HCDR3 comprises an amino acid sequence of SEQ ID NO: 9, or a variant thereof, and
   (iv) a VL comprising LCDR1, LCDR2, and LCDR3 sequences, wherein the LCDR1 comprises an amino acid sequence of SEQ ID NO: 37, or a variant thereof, the LCDR2 comprises an amino acid sequence of SEQ ID NO: 38, or a variant thereof, and the LCDR3 comprises an amino acid sequence of SEQ ID NO: 39, or a variant thereof,
XIII.
(a) an amino acid sequence of SEQ ID NO: 109, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 1, or a variant thereof, the LCDR2 comprises an amino acid sequence of SEQ ID NO: 2, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 6, or a variant thereof,
   (iii) a VH comprising HCDR1, HCDR2, and HCDR3 sequences, wherein the HCDR1 comprises an amino acid sequence of SEQ ID NO: 40, or a variant thereof, the HCDR2 comprises an amino acid sequence of SEQ ID NO: 41, or a variant thereof, and the HCDR3 comprises an amino acid sequence of SEQ ID NO: 42, or a variant thereof, and
   (iv) a VL comprising LCDR1, LCDR2, and LCDR3 sequences, wherein the LCDR1 comprises an amino acid sequence of SEQ ID NO: 43, or a variant thereof, the LCDR2 comprises an amino acid sequence of SEQ ID NO: 44, or a variant thereof, and the LCDR3 comprises an amino acid sequence of SEQ ID NO: 45, or a variant thereof, and
XIV.
(a) an amino acid sequence of SEQ ID NO: 110, or an amino acid sequence having at least about 90%, or about 93%, or about 95%, or about 97%, or about 98%, or about 99% identity thereto, or
(b)
   (i) a VL comprising LCDR1, LCDR2, and LCDR3 sequences, wherein the LCDR1 comprises an amino acid sequence of SEQ ID NO: 13, or a variant thereof, the LCDR2 comprises an amino acid sequence of SEQ ID NO: 14, or a variant thereof, and the LCDR3 comprises an amino acid sequence of SEQ ID NO: 3, or a variant thereof,
   (ii) a VH comprising HCDR1, HCDR2, and HCDR3 sequences, wherein the HCDR1 comprises an amino acid sequence of SEQ ID NO: 4, or a variant thereof, the HCDR2 comprises an amino acid sequence of SEQ ID NO: 5, or a variant thereof, and the HCDR3 comprises an amino acid sequence of SEQ ID NO: 15, or a variant thereof,
   (iii) a VH comprising HCDR1, HCDR2, and HCDR3 sequences, wherein the HCDR1 comprises an amino acid sequence of SEQ ID NO: 40, or a variant thereof, the HCDR2 comprises an amino acid sequence of SEQ ID NO: 41, or a variant thereof, and the HCDR3 comprises an amino acid sequence of SEQ ID NO: 42, or a variant thereof, and
   (iv) a VL comprising LCDR1, LCDR2, and LCDR3 sequences, wherein the LCDR1 comprises an amino acid sequence of SEQ ID NO: 43, or a variant thereof, the LCDR2 comprises an amino acid sequence of SEQ ID NO: 44, or a variant thereof, and the LCDR3 comprises an amino acid sequence of SEQ ID NO: 45, or a variant thereof, or
wherein:
   any of the above sequences in subsections (a) optionally can have a His6 tag (e.g., HHHHHH (SEQ ID NO: 46)) added to the N or C-terminus and
   any of the above sequences optionally have the linker comprising one or more glycines and serines replaced with another peptide linker or functionally equivalent variation thereof.

2. A composition comprising a scFv-Fab Fe antibody specific for claudin 6 and CD3 comprising:
(a) a first heavy chain selected from:
(i) SEQ ID NO: 79,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(ii) SEQ ID NO: 88,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
   or
(iii) SEQ ID NO: 114,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof,
(b) a second heavy chain selected from:
(i.) SEQ ID NO: 80,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof, or
(ii.) SEQ ID NO: 89,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 26, or a variant thereof, and the heavy chain CDR3 comprises an amino acid sequence of SEQ ID NO: 27, or a variant thereof, or
   a heavy chain variable region comprising heavy chain CDR1, CDR2, and CDR3 sequences, wherein the heavy chain CDR1 comprises an amino acid sequence of SEQ ID NO: 28, or a variant thereof, the heavy chain CDR2 comprises an amino acid sequence of SEQ ID NO: 29, or a variant thereof, and the heavy chain CDR3 comprises an amino acid sequence of SEQ ID NO: 30, or a variant thereof, or
(iii.) SEQ ID NO: 90,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 31, or a variant thereof, and the heavy chain CDR3 comprises an amino acid sequence of SEQ ID NO: 32, or a variant thereof, or
   a heavy chain variable region comprising heavy chain CDR1, CDR2, and CDR3 sequences, wherein the heavy chain CDR1 comprises an amino acid sequence of SEQ ID NO: 48, or a variant thereof, the heavy chain CDR2 comprises an amino acid sequence of SEQ ID NO: 34, or a variant thereof, and the heavy chain CDR3 comprises an amino acid sequence of SEQ ID NO: 35, or a variant thereof, or
(iv.) SEQ ID NO: 91,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 19, or a variant thereof, the heavy chain CDR2 comprises an amino acid sequence of SEQ ID NO: 20, or a variant thereof, and the heavy chain CDR3 comprises an amino acid sequence of SEQ ID NO: 21, or a variant thereof, or
   a heavy chain variable region comprising heavy chain CDR1, CDR2, and CDR3 sequences, wherein the heavy chain CDR1 comprises an amino acid sequence of SEQ ID NO: 49, or a variant thereof, the heavy chain CDR2 comprises an amino acid sequence of SEQ ID NO: 23, or a variant thereof, and the heavy chain CDR3 comprises an amino acid sequence of SEQ ID NO: 24, or a variant thereof, or
(v.) SEQ ID NO: 92,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 7, or a variant thereof, the heavy chain CDR2 comprises an amino acid sequence of SEQ ID NO: 36, or a variant thereof, and the heavy chain CDR3 comprises an amino acid sequence of SEQ ID NO: 9, or a variant thereof, or
   a heavy chain variable region comprising heavy chain CDR1, CDR2, and CDR3 sequences, wherein the heavy chain CDR1 comprises an amino acid sequence of SEQ ID NO: 37, or a variant thereof, the heavy chain CDR2 comprises an amino acid sequence of SEQ ID NO: 38, or a variant thereof, and the heavy chain CDR3 comprises an amino acid sequence of SEQ ID NO: 39, or a variant thereof, or
(vi.) SEQ ID NO: 111,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 40, or a variant thereof, the heavy chain CDR2 comprises an amino acid sequence of SEQ ID NO: 41, or a variant thereof, and the heavy chain CDR3 comprises an amino acid sequence of SEQ ID NO: 42, or a variant thereof, or
   a heavy chain variable region comprising heavy chain CDR1, CDR2, and CDR3 sequences, wherein the heavy chain CDR1 comprises an amino acid sequence of SEQ ID NO: 43, or a variant thereof, the heavy chain CDR2 comprises an amino acid sequence of SEQ ID NO: 44, or a variant thereof, and the heavy chain CDR3 comprises an amino acid sequence of SEQ ID NO: 45, or a variant thereof,
and
(c) a first light chain selected from:
(i) SEQ ID NO: 67,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
or
(iii) SEQ ID NO: 65,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

3. The composition of embodiment 2, wherein the Fc is from IgG.

4. The composition of embodiment 3, wherein the IgG is human IgG.

5. The composition of embodiment 4, wherein the human IgG is selected from IgG1, IgG2, IgG3, and IgG4.

6. The composition of any one of embodiments 2 to 5, wherein the antibody is formed through a knob-in-hole interaction in the Fc region.

7. The composition of embodiment 4 or embodiment 5, wherein the human IgG Fc comprises one or mutations to promote knob-in-hole interaction.

8. The composition of embodiment 7, wherein the mutations are selected from (i) T366Y or T366W, and (ii) Y407T, Y407A, or Y407V.

9. The composition of embodiment 7, wherein the mutations are selected from:
(a) T366Y and Y407T or T366Y/F405A and T394W/Y407T in human IgG1, or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

10. The composition of embodiment 4, wherein the human IgG Fc comprises one or mutations to reduce or eliminate the effector function of the Fc domains.

11. The composition of embodiment 10, wherein the mutations are L234A and L235A (LALA) substitutions in human IgG1.

12. The composition of embodiment 4, wherein the human IgG Fc comprises one or mutations to stabilize a hinge region in the Fc domain.

13. The composition of embodiment 12, wherein the mutation is S228P.

14. A composition comprising an IgG-(scFV)₂ antibody specific for claudin 6 and CD3 comprising:
(a) a heavy chain selected from:
   (i.) SEQ ID NO: 86,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
   (ii.) SEQ ID NO: 87,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
   (iii.) SEQ ID NO: 101,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 7, or a variant thereof, the heavy chain CDR2 comprises an amino acid sequence of SEQ ID NO: 36, or a variant thereof, and the heavy chain CDR3 comprises an amino acid sequence of SEQ ID NO: 9, or a variant thereof, or
   a heavy chain variable region comprising heavy chain CDR1, CDR2, and CDR3 sequences, wherein the heavy chain CDR1 comprises an amino acid sequence of SEQ ID NO: 37, or a variant thereof, the heavy chain CDR2 comprises an amino acid sequence of SEQ ID NO: 38, or a variant thereof, and the heavy chain CDR3 comprises an amino acid sequence of SEQ ID NO: 39, or a variant thereof,
   (iv.) SEQ ID NO: 102,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 7, or a variant thereof, the heavy chain CDR2 comprises an amino acid sequence of SEQ ID NO: 36, or a variant thereof, and the heavy chain CDR3 comprises an amino acid sequence of SEQ ID NO: 9, or a variant thereof, or
   a heavy chain variable region comprising heavy chain CDR1, CDR2, and CDR3 sequences, wherein the heavy chain CDR1 comprises an amino acid sequence of SEQ ID NO: 37, or a variant thereof, the heavy chain CDR2 comprises an amino acid sequence of SEQ ID NO: 38, or a variant thereof, and the heavy chain CDR3 comprises an amino acid sequence of SEQ ID NO: 39, or a variant thereof,
   (v.) SEQ ID NO: 103,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 26, or a variant thereof, and the heavy chain CDR3 comprises an amino acid sequence of SEQ ID NO: 27, or a variant thereof, or
   a heavy chain variable region comprising heavy chain CDR1, CDR2, and CDR3 sequences, wherein the heavy chain CDR1 comprises an amino acid sequence of SEQ ID NO: 28, or a variant thereof, the heavy chain CDR2 comprises an amino acid sequence of SEQ ID NO: 29, or a variant thereof, and the heavy chain CDR3 comprises an amino acid sequence of SEQ ID NO: 30, or a variant thereof,
   (vi.) SEQ ID NO: 104,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 26, or a variant thereof, and the heavy chain CDR3 comprises an amino acid sequence of SEQ ID NO: 27, or a variant thereof, or
   a heavy chain variable region comprising heavy chain CDR1, CDR2, and CDR3 sequences, wherein the heavy chain CDR1 comprises an amino acid sequence of SEQ ID NO: 28, or a variant thereof, the heavy chain CDR2 comprises an amino acid sequence of SEQ ID NO: 29, or a variant thereof, and the heavy chain CDR3 comprises an amino acid sequence of SEQ ID NO: 30, or a variant thereof,
   (vii.) SEQ ID NO: 105,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 31, or a variant thereof, and the heavy chain CDR3 comprises an amino acid sequence of SEQ ID NO: 32, or a variant thereof, or
   a heavy chain variable region comprising heavy chain CDR1, CDR2, and CDR3 sequences, wherein the heavy chain CDR1 comprises an amino acid sequence of SEQ ID NO: 33, or a variant thereof, the heavy chain CDR2 comprises an amino acid sequence of SEQ ID NO: 34, or a variant thereof, and the heavy chain CDR3 comprises an amino acid sequence of SEQ ID NO: 35, or a variant thereof,
   (viii.) SEQ ID NO: 106,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 31, or a variant thereof, and the heavy chain CDR3 comprises an amino acid sequence of SEQ ID NO: 32, or a variant thereof, or
   a heavy chain variable region comprising heavy chain CDR1, CDR2, and CDR3 sequences, wherein the heavy chain CDR1 comprises an amino acid sequence of SEQ ID NO: 33, or a variant thereof, the heavy chain CDR2 comprises an amino acid sequence of SEQ ID NO: 34, or a variant thereof, and the heavy chain CDR3 comprises an amino acid sequence of SEQ ID NO: 35, or a variant thereof,
   (ix.) SEQ ID NO: 107,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 19, or a variant thereof, the heavy chain CDR2 comprises an amino acid sequence of SEQ ID NO: 20, or a variant thereof, and the heavy chain CDR3 comprises an amino acid sequence of SEQ ID NO: 21, or a variant thereof, or
   a heavy chain variable region comprising heavy chain CDR1, CDR2, and CDR3 sequences, wherein the heavy chain CDR1 comprises an amino acid sequence of SEQ ID NO: 22, or a variant thereof, the heavy chain CDR2 comprises an amino acid sequence of SEQ ID NO: 23, or a variant thereof, and the heavy chain CDR3 comprises an amino acid sequence of SEQ ID NO: 24, or a variant thereof,
   (x.) SEQ ID NO: 108,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 19, or a variant thereof, the heavy chain CDR2 comprises an amino acid sequence of SEQ ID NO: 20, or a variant thereof, and the heavy chain CDR3 comprises an amino acid sequence of SEQ ID NO: 21, or a variant thereof, or
   a heavy chain variable region comprising heavy chain CDR1, CDR2, and CDR3 sequences, wherein the heavy chain CDR1 comprises an amino acid sequence of SEQ ID NO: 22, or a variant thereof, the heavy chain CDR2 comprises an amino acid sequence of SEQ ID NO: 23, or a variant thereof, and the heavy chain CDR3 comprises an amino acid sequence of SEQ ID NO: 24, or a variant thereof,
   (xi.) SEQ ID NO: 112,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof, or
   a heavy chain variable region comprising heavy chain CDR1, CDR2, and CDR3 sequences, wherein the heavy chain CDR1 comprises an amino acid sequence of SEQ ID NO: 40, or a variant thereof, the heavy chain CDR2 comprises an amino acid sequence of SEQ ID NO: 41, or a variant thereof, and the heavy chain CDR3 comprises an amino acid sequence of SEQ ID NO: 42, or a variant thereof, or
   a heavy chain variable region comprising heavy chain CDR1, CDR2, and CDR3 sequences, wherein the heavy chain CDR1 comprises an amino acid sequence of SEQ ID NO: 43, or a variant thereof, the heavy chain CDR2 comprises an amino acid sequence of SEQ ID NO: 44, or a variant thereof, and the heavy chain CDR3 comprises an amino acid sequence of SEQ ID NO: 45, or a variant thereof,
   (xii.) SEQ ID NO: 113,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence is of SEQ ID NO: 5, or a variant thereof, and the heavy chain CDR3 comprises an amino acid sequence of SEQ ID NO: 15, or a variant thereof, or
   a heavy chain variable region comprising heavy chain CDR1, CDR2, and CDR3 sequences, wherein the heavy chain CDR1 comprises an amino acid sequence of SEQ ID NO: 40, or a variant thereof, the heavy chain CDR2 comprises an amino acid sequence of SEQ ID NO: 41, or a variant thereof, and the heavy chain CDR3 comprises an amino acid sequence of SEQ ID NO: 42, or a variant thereof, or
   a heavy chain variable region comprising heavy chain CDR1, CDR2, and CDR3 sequences, wherein the heavy chain CDR1 comprises an amino acid sequence of SEQ ID NO: 43, or a variant thereof, the heavy chain CDR2 comprises an amino acid sequence of SEQ ID NO: 44, or a variant thereof, and the heavy chain CDR3 comprises an amino acid sequence of SEQ ID NO: 45, or a variant thereof,
   and
(b) a light chain selected from:
   (i) SEQ ID NO: 67,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
   (ii) SEQ ID NO: 66,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
   or
   (iii) SEQ ID NO: 65,
   an amino acid sequence having at least about 90%, or about 93%, or about 95%, or about 97%, or about 98%, or about 99% identity thereto, or
   a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, or
   wherein:
      any of the above sequences in subsections (a) optionally can have a His6 tag (e.g., HHHHHH (SEQ ID NO: 46)) added to the N or C-terminus and
   any of the above sequences optionally have the linker comprising one or more glycines and serines replaced with another peptide linker or functionally equivalent variation thereof.

15. The composition of embodiment 14, wherein the Fc is from IgG.

16. The composition of embodiment 15, wherein the IgG is human IgG.

17. The composition of embodiment 16, wherein the human IgG is selected from IgG1, IgG2, IgG3, and IgG4.

18. The composition of embodiment 16, wherein the human IgG Fc comprises one or mutations to reduce or eliminate the effector function of the Fc domains.

19. The composition of embodiment 18, wherein the mutations are L234A and L235A (LALA) substitutions in human IgG1.

20. The composition of embodiment 16, wherein the human IgG Fc comprises one or mutations to stabilize a hinge region in the Fc domain.

21. The composition of embodiment 20, wherein the mutation is S228P.

22. A composition comprising a scFv-Fab Fe antibody specific for claudin 6 and CD3 comprising:
(a) a first heavy chain selected from:
(i) SEQ ID NO: 79,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(ii) SEQ ID NO: 88,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
   or
(iii) SEQ ID NO: 114,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof,
(b) a second heavy chain selected from:
SEQ ID NO: 81,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
and
(c) a light chain selected from:
(i) SEQ ID NO: 67,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
or
(iii) SEQ ID NO: 65,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

23. The composition of embodiment 22, wherein the Fc is from IgG.

24. The composition of embodiment 23, wherein the IgG is human IgG.

25. The composition of embodiment 24, wherein the human IgG is selected from IgG1, IgG2, IgG3, and IgG4.

26. The composition of any one of embodiments 22 to 25, wherein the antibody is formed through a knob-in-hole interaction in the Fc region.

27. The composition of embodiment 24, wherein the human IgG Fc comprises one or mutations to promote knob-in-hole interaction.

28. The composition of embodiment 27, wherein the mutations are selected from (i) T366Y or T366W, and (ii) Y407T, Y407A, or Y407V.

29. The composition of embodiment 24, wherein the mutations are:
(a) T366Y and Y407T or T366Y/F405A and T394W/Y407T in human IgG1 or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

30. The composition of embodiment 24, wherein the human IgG Fc comprises one or mutations to reduce or eliminate the effector function of the Fc domains.

31. The composition of embodiment 30, wherein the mutations are L234A and L235A (LALA) substitutions in human IgG1.

32. The composition of embodiment 24, wherein the human IgG Fc comprises one or mutations to stabilize a hinge region in the Fc domain.

33. The composition of embodiment 32, wherein the mutation is S228P.

34. A composition comprising a scFv-Fab Fc antibody specific for claudin 6 and CD3 comprising:
(a) a first heavy chain selected from:
(i) SEQ ID NO: 79,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(ii) SEQ ID NO: 88,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof, or
(iii) SEQ ID NO: 114,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof,
(b) a second heavy chain selected from:
(i.) SEQ ID NO: 117,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
(ii.) SEQ ID NO: 119,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
(iii.) SEQ ID NO: 118,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(iv.) SEQ ID NO: 120,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 16, or a variant thereof, the heavy chain CDR2 comprises an amino acid sequence of SEQ ID NO: 17, or a variant thereof, and the heavy chain CDR3 comprises an amino acid sequence of SEQ ID NO: 18, or a variant thereof,
or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
and
(c) a light chain selected from:
(i) SEQ ID NO: 67,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
or
(iii) SEQ ID NO: 65,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

35. The composition of embodiment 34, wherein the Fc is from IgG.

36. The composition of embodiment 35, wherein the IgG is human IgG.

37. The composition of embodiment 36, wherein the human IgG is selected from IgG1, IgG2, IgG3, and IgG4.

38. The composition of any one of embodiments 34 to 37, wherein the antibody is formed through a knob-in-hole interaction in the Fc region.

39. The composition of embodiment 36, wherein the human IgG Fc comprises one or mutations to promote knob-in-hole interaction.

40. The composition of embodiment 39, wherein the mutations are selected from (i) T366Y or T366W, and (ii) Y407T, Y407A, or Y407V.

41. The composition of embodiment 36, wherein the mutations are:
(a) T366Y and Y407T or T366Y/F405A and T394W/Y407T in human IgG1 or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

42. The composition of embodiment 36, wherein the human IgG Fc comprises one or mutations to reduce or eliminate the effector function of the Fc domains.

43. The composition of embodiment 42, wherein the mutations are L234A and L235A (LALA) substitutions in human IgG1.

44. The composition of embodiment 36, wherein the human IgG Fc comprises one or mutations to stabilize a hinge region in the Fc domain.

45. The composition of embodiment 44, wherein the mutation is S228P.

46. A composition comprising a scFv-Fab Fc antibody specific for claudin 6 and CD3 comprising:
(a) a first heavy chain selected from:
(i) SEQ ID NO: 79,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 6, or a variant thereof,
(ii) SEQ ID NO: 88,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 15, or a variant thereof,
or
(iii) SEQ ID NO: 114,
   an amino acid sequence having at least about 90%, or about 93%, or about 95%, or about 97%, or about 98%, or about 99% identity thereto, or
   a heavy chain variable region comprising heavy chain CDR1, CDR2, and CDR3 sequences, wherein the heavy chain CDR1 comprises an amino acid sequence of SEQ ID NO: 4, or a variant thereof, the heavy chain CDR2 comprises an amino acid sequence of SEQ ID NO: 5, or a variant thereof, and the heavy chain CDR3 comprises an amino acid sequence of SEQ ID NO: 47, or a variant thereof,
(b) a second heavy chain selected from:
(i) SEQ ID NO: 121,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 26, or a variant thereof, and the heavy chain CDR3 comprises an amino acid sequence of SEQ ID NO: 27, or a variant thereof,
(ii) SEQ ID NO: 123,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 25, or a variant thereof, the heavy chain CDR2 comprises an amino acid sequence of SEQ ID NO: 31, or a variant thereof, and the heavy chain CDR3 comprises an amino acid sequence of SEQ ID NO: 32, or a variant thereof,
(iii) SEQ ID NO: 125,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 19, or a variant thereof, the heavy chain CDR2 comprises an amino acid sequence of SEQ ID NO: 20, or a variant thereof, and the heavy chain CDR3 comprises an amino acid sequence of SEQ ID NO: 21, or a variant thereof,
(iv) SEQ ID NO: 127,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 7, or a variant thereof, the heavy chain CDR2 comprises an amino acid sequence of SEQ ID NO: 36, or a variant thereof, and the heavy chain CDR3 comprises an amino acid sequence of SEQ ID NO: 9, or a variant thereof,
(v) SEQ ID NO: 129,
an amino acid sequence having at least about 90%, or about 93%, or about 95%, or about 97%, or about 98%, or about 99% identity thereto, or
a heavy chain variable region comprising heavy chain CDR1, CDR2, and CDR3 sequences, wherein the heavy chain CDR1 comprises an amino acid sequence of SEQ ID NO: 40, or a variant thereof, the heavy chain CDR2 comprises an amino acid sequence of SEQ ID NO: 41, or a variant thereof, and the heavy chain CDR3 comprises an amino acid sequence of SEQ ID NO: 42, or a variant thereof,
and
(c) a first light chain selected from:
(i) SEQ ID NO: 67,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof,
(ii) SEQ ID NO: 66,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 13, or a variant thereof, the light chain CDR2 comprises an amino acid sequence of SEQ ID NO: 14, or a variant thereof, and the light chain CDR3 comprises an amino acid sequence of SEQ ID NO: 3, or a variant thereof,
or
(iii) SEQ ID NO: 65,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 1, or a variant thereof, the light chain CDR2 comprises an amino acid sequence of SEQ ID NO: 2, or a variant thereof, and the light chain CDR3 comprises an amino acid sequence of SEQ ID NO: 3, or a variant thereof
and
(d) a second light chain selected from:
(i) SEQ ID NO: 122,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 28, or a variant thereof, the light chain CDR2 comprises an amino acid sequence of SEQ ID NO: 29, or a variant thereof, and the light chain CDR3 comprises an amino acid sequence of SEQ ID NO: 30, or a variant thereof, or
(ii) SEQ ID NO: 124,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 33, or a variant thereof, the light chain CDR2 comprises an amino acid sequence of SEQ ID NO: 34, or a variant thereof, and the light chain CDR3 comprises an amino acid sequence of SEQ ID NO: 35, or a variant thereof, or
(iii) SEQ ID NO: 126,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 22, or a variant thereof, the light chain CDR2 comprises an amino acid sequence of SEQ ID NO: 23, or a variant thereof, and the light chain CDR3 comprises an amino acid sequence of SEQ ID NO: 24, or a variant thereof, or
(iv) SEQ ID NO: 128,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 37, or a variant thereof, the light chain CDR2 comprises an amino acid sequence of SEQ ID NO: 38, or a variant thereof, and the light chain CDR3 comprises an amino acid sequence of SEQ ID NO: 39, or a variant thereof, or
(v) SEQ ID NO: 130,
an amino acid sequence having at least about 90%, or about 93%, or about 95%, or about 97%, or about 98%, or about 99% identity thereto, or
a light chain variable region comprising light chain CDR1, CDR2, and CDR3 sequences, wherein the light chain CDR1 comprises an amino acid sequence of SEQ ID NO: 43, or a variant thereof, the light chain CDR2 comprises an amino acid sequence of SEQ ID NO: 44, or a variant thereof, and the light chain CDR3 comprises an amino acid sequence of SEQ ID NO: 45, or a variant thereof, or
wherein:
   any of the above sequences in subsections (a) optionally can have a His6 tag (e.g., HHHHHH (SEQ ID NO: 46)) added to the N or C-terminus and
any of the above sequences optionally have the linker comprising one or more glycines and serines replaced with another peptide linker or functionally equivalent variation thereof.

47. The composition of embodiment 46, wherein the Fc is from IgG.

48. The composition of embodiment 47, wherein the IgG is human IgG.

49. The composition of embodiment 48, wherein the human IgG is selected from IgG1, IgG2, IgG3, and IgG4.

50. The composition of any one of embodiments 46 to 49, wherein the antibody is formed through a knob-in-hole interaction in the Fc region.

51. The composition of embodiment 46, wherein the human IgG Fc comprises one or mutations to promote knob-in-hole interaction.

52. The composition of embodiment 51, wherein the mutations are selected from (i) T366Y or T366W, and (ii) Y407T, Y407A, or Y407V.

53. The composition of embodiment 51, wherein the mutations are:
(a) T366Y and Y407T or T366Y/F405A and T394W/Y407T in human IgG1 or
(b) T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V,
in human IgG1.

54. The composition of embodiment 46, wherein the human IgG Fc comprises one or mutations to reduce or eliminate the effector function of the Fc domains.

55. The composition of embodiment 54, wherein the mutations are L234A and L235A (LALA) substitutions in human IgG1.

56. The composition of embodiment 46, wherein the human IgG Fc comprises one or mutations to stabilize a hinge region in the Fc domain.

57. The composition of embodiment 56, wherein the mutation is S228P.

58. The composition of any one of embodiments 1 to 57, wherein the composition binds claudin 6 and CD3 contemporaneously.

59. The composition of any one of embodiments 1 to 58, wherein the composition binds claudin 6 with an affinity of less than 10 nM and with at least 100 fold greater affinity than claudin 9, claudin 3, and/or claudin 4.

60. A pharmaceutical composition comprising an isolated antibody of any one of embodiments 1 to 59, or a nucleic acid molecule encoding the same.

61. The pharmaceutical composition of embodiment 60, wherein the composition is an injectable pharmaceutical composition.

62. The pharmaceutical composition of embodiments 60 or 61, wherein the composition is sterile.

63. The pharmaceutical compositions of any one of embodiments 60 to 62, wherein the composition is pyrogen free.

64. A nucleic acid molecule encoding an antibody or an amino acid sequence of any of the preceding embodiments.

65. A vector comprising the nucleic acid molecule of embodiment 64.

66. A cell comprising the nucleic acid molecule of embodiment 64, or the vector of embodiment 65.

67. A method for modulating and/or targeting claudin 6 and CD3 in a biological cell, comprising contacting the cell with a composition of any one of embodiments 1 to 59.

68. A method for modulating claudin 6 activity in a biological cell comprising contacting a cell expressing claudin 6 with a composition of any one of embodiments 1 to 59.

69. A method for inhibiting the function of claudin 6 in a biological cell comprising contacting a cell expressing claudin 6 with a composition of any one of embodiments 1 to 59.

70. A method for treating or preventing cancer comprising administering an effective amount of the composition of any one of embodiments 1 to 59 to a subject in need thereof.

71. A use of the composition of any one of embodiments 1 to 59 for the preparation of a medicament for the treatment of prevention of cancer.

72. The method or use of any one of embodiments 1 to 71, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; glioma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; malignant rhabdoid tumor; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

73. An isolated antibody comprising one or more of the sequences disclosed herein.

74. A bispecific antibody comprising three polypeptides, the bispecific antibody comprising:
a first polypeptide comprising a first light chain comprising a first variable light chain region, wherein the first variable light chain region comprises:
   (1) a CDR1 sequence comprising the amino acid sequence of SEQ ID NO: 1, or a variant thereof;
   (2) a CDR2 sequence comprising the amino acid sequence of SEQ ID NO: 2, or a variant thereof; and
   (3) a CDR3 sequence comprising the amino acid sequence of SEQ ID NO: 3 or a variant thereof;
a second polypeptide comprising a first heavy chain comprising a first variable region heavy chain region, wherein the first variable heavy chain region comprises:
   (1) a CDR1 comprising the amino acid sequence of SEQ ID NO: 4, or a variant thereof;
   (2) a CDR2 comprising the amino acid sequence of SEQ ID NO: 5, or a variant thereof, and
   (3) a CDR3 comprising the amino acid sequence of SEQ ID NO: 6, or a variant thereof; and
a third polypeptide comprising a second light chain and a second a heavy chain, wherein the second heavy chain comprises a second variable heavy chain region comprising:
   (1) a CDR1 comprising the amino acid sequence of SEQ ID NO: 25, or a variant thereof;
   (2) a CDR2 comprising the amino acid sequence of SEQ ID NO: 26, or a variant thereof; and
   (3) a CDR3 comprising the amino acid sequence of SEQ ID NO: 27, or a variant thereof; and
   the second light chain comprises a second variable light chain region comprising:
   (1) a CDR1 comprising the amino sequence of SEQ ID NO: 28, or a variant thereof;
   (2) a CDR2 comprising the amino acid sequence of SEQ ID NO: 29, or a variant thereof; and
   (3) a CDR3 comprising the amino acid sequence of SEQ ID NO: 30, or a variant thereof.

75. The bispecific antibody of embodiment 74, wherein the first variable light chain region comprises the amino acid sequence of SEQ ID NO: 68, or an amino acid sequence having at least about 90%, or about 93%, or about 95%, or about 97%, or about 98%, or about 99% identity thereto.

76. The bispecific antibody of embodiments 74 or 75, wherein the first light chain comprises the first variable light chain region and a light chain constant domain.

77. The bispecific antibody of embodiment 76, wherein the first light chain constant domain comprises the amino acid sequence of SEQ ID NO: 72, or an amino acid sequence having at least about 90%, or about 93%, or about 95%, or about 97%, or about 98%, or about 99% identity thereto.

78. The bispecific antibody of any one of embodiments 74-77, wherein the first polypeptide comprises the amino acid sequence of SEQ ID NO: 67, or an amino acid sequence having at least about 90%, or about 93%, or about 95%, or about 97%, or about 98%, or about 99% identity thereto.

79. The bispecific antibody of any one of embodiments 74-78, wherein the first variable heavy chain region comprises the amino acid sequence of SEQ ID NO: 69, or an amino acid sequence having at least about 90%, or about 93%, or about 95%, or about 97%, or about 98%, or about 99% identity thereto.

80. The bispecific antibody of any one of embodiments 74-79, wherein the first heavy chain comprises the first heavy chain variable region and a first heavy chain constant domain.

81. The bispecific antibody of embodiment 80, wherein the first heavy chain constant domain comprises the amino acid sequence of SEQ ID NO: 73, or an amino acid sequence having at least about 90%, or about 93%, or about 95%, or about 97%, or about 98%, or about 99% identity thereto.

82. The bispecific antibody of any one of embodiments 74-81, wherein the second polypeptide comprises the amino acid sequence of SEQ ID NO: 79, or an amino acid sequence having at least about 90%, or about 93%, or about 95%, or about 97%, or about 98%, or about 99% identity thereto.

83. The bispecific antibody of any one of embodiments 74-82, wherein the second variable heavy chain region comprises the amino acid sequence of SEQ ID NO: 70, or an amino acid sequence having at least about 90%, or about 93%, or about 95%, or about 97%, or about 98%, or about 99% identity thereto.

84. The bispecific antibody of any one of embodiments 74-83, wherein the second variable light chain region comprises the amino acid sequence of SEQ ID NO: 71, or an amino acid sequence having at least about 90%, or about 93%, or about 95%, or about 97%, or about 98%, or about 99% identity thereto.

85. The bispecific antibody of any one of embodiments 74-84, wherein the second variable heavy chain region and the second variable light chain region are linked by a peptide linker, or optionally the linker comprising one or more glycines and serines is replaced with another peptide linker, or a functionally equivalent variation thereof.

86. The bispecific antibody of embodiment 85, wherein the linker comprises the amino sequence of SEQ ID NO: 53, or a variant thereof.

87. The bispecific antibody of any one of embodiments 74-86, wherein the third polypeptide comprises a constant domain.

88. The bispecific antibody of embodiment 87, wherein the constant domain is linked to the C-terminus of the second variable light chain region.

89. The bispecific antibody of embodiment 87 or 88, wherein there is no peptide linker between the C-terminus of the second variable light chain region and the constant domain.

90. The bispecific antibody of any one of embodiments 87-89, wherein the constant domain comprises the amino acid sequence of SEQ ID NO: 74, or an amino acid sequence having at least about 90%, or about 93%, or about 95%, or about 97%, or about 98%, or about 99% identity thereto.

91. The bispecific antibody of any one of embodiments 74-90, wherein the third polypeptide comprises the amino acid sequence of SEQ ID NO: 89, or an amino acid sequence having at least about 90%, or about 93%, or about 95%, or about 97%, or about 98%, or about 99% identity thereto.

92. The bispecific antibody of any one of embodiments 74-91, wherein:
the first polypeptide comprises the amino acid sequence of SEQ ID NO: 67, or an amino acid sequence having at least about 90%, or about 93%, or about 95%, or about 97%, or about 98%, or about 99% identity thereto;
the second polypeptide comprises the amino acid sequence of SEQ ID NO: 79, or an amino acid sequence having at least about 90%, or about 93%, or about 95%, or about 97%, or about 98%, or about 99% identity thereto; and
the third polypeptide comprises the amino acid sequence of SEQ ID NO: 89, or an amino acid sequence having at least about 90%, or about 93%, or about 95%, or about 97%, or about 98%, or about 99% identity thereto.

93. The bispecific antibody of any one of embodiments 80, 87, 88, or 89, wherein the constant domain comprises a human IgG Fc comprises a knob-in-hole interaction mutation.

94. The bispecific antibody of embodiment 93, wherein the mutations are selected from (i) T366Y or T366W, and (ii) Y407T, Y407A, or Y407V.

95. The bispecific antibody of embodiment 93, wherein the mutations are:
T366Y and Y407T or T366Y/F405A and T394W/Y407T;
T366W/D399C and T366S/L368A/K392C/Y407V;
T366W/K392C and T366S/L368A/D399C/Y407V;
S354C/T366W and Y349C/T366S/L368A/Y407V;
Y349C/T366W and S354C/T366S/L368A/Y407V;
E356C/T366W and Y349C/T366S/L368A/Y407V;
Y349C/T366W and E356C/T366S/L368A/Y407V;
E357C/T366W and Y349C/T366S/L368A/Y407V; or
Y349C/T366W and E357C/T366S/L368A/Y407V; and
wherein the numbering is according to the EU numbering in human IgG1.

96. The bispecific antibody of any one of embodiments 80, 87, 88, or 89, wherein the constant domain comprises one or more mutations to reduce or eliminate the effector function of the constant domain.

97. The bispecific antibody of embodiment 96, wherein the mutations are L234A and L235A (LALA) substitutions, wherein the numbering is according to the EU numbering in human IgG1 in human IgG1.

98. The bispecific antibody any one of embodiments 80, 87, 88, or 89, wherein the constant comprises one or more mutations to stabilize a hinge region in the constant domain.

99. The bispecific antibody of embodiment 98, wherein the mutation is S228P.

100. The bispecific antibody of any of embodiments 74-99, wherein the bispecific antibody binds claudin 6 and CD3 contemporaneously.

101. The bispecific antibody of any one of embodiments 74-100, wherein the bispecific antibody binds claudin 6 with an affinity of less than about 10 nM and/or with at least about 100 fold greater affinity than claudin 9, claudin 3, and/or claudin 4.

102. A pharmaceutical composition comprising the bispecific antibody of any one of embodiments 74-101, or a nucleic acid molecule encoding the same.

103. The pharmaceutical composition of embodiment 102, wherein the composition is an injectable pharmaceutical composition, optionally a composition suitable for intravenous injection (administration) or subcutaneous injection (administration).

104. A nucleic acid molecule encoding the polypeptides of the bispecific antibody of any one of embodiments 74-101.

105. A plurality of nucleic acid molecules encoding the polypeptides of the bispecific antibody of any one of embodiments 74-101.

106. A vector comprising the nucleic acid molecule or molecules of embodiments 104 or 105.

107. A cell comprising the nucleic acid molecule of embodiments 104 or 105, or the vector of embodiment 106.

108. A method for modulating and/or targeting claudin 6 and CD3 in a biological cell, comprising contacting the cell with a bispecific antibody of any one of embodiments 74-101 or the pharmaceutical composition of embodiments 102 or 103.

109. A method for modulating claudin 6 activity in a biological cell comprising contacting a cell expressing claudin 6 with a bispecific of any one of embodiments 74-101 or the pharmaceutical composition of embodiments 102 or 103.

110. A method for inhibiting the function of claudin 6 in a biological cell comprising contacting a cell expressing claudin 6 with a bispecific antibody of any one of embodiments 74-101 or the pharmaceutical composition of embodiments 102 or 103.

111. A method for treating or preventing cancer comprising administering an effective amount of the bispecific antibody of any one of embodiments of any one of embodiments 74-101 or the pharmaceutical composition of embodiments 102 or 103.

112. A use of the bispecific antibody of any one of embodiments 74-101 or the pharmaceutical composition of embodiments 102 or 103 in the preparation of a medicament for the treatment or prevention of cancer.

113. A pharmaceutical composition for use in treating cancer, wherein the pharmaceutical composition comprises a bispecific antibody of any one of embodiments 74-101.

114. The method, use, or pharmaceutical composition of any one of embodiments 111-113, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

115. The method or use of embodiment 114, wherein the cancer is non-small cell lung (NSCLC), ovarian, gastric, breast, endometrial, or testicular cancer.

This disclosure is further illustrated by the following non-limiting examples.

### EXAMPLES

### Example 1 Overview and Development of Antibodies Specific for Claudin 6 and CD3

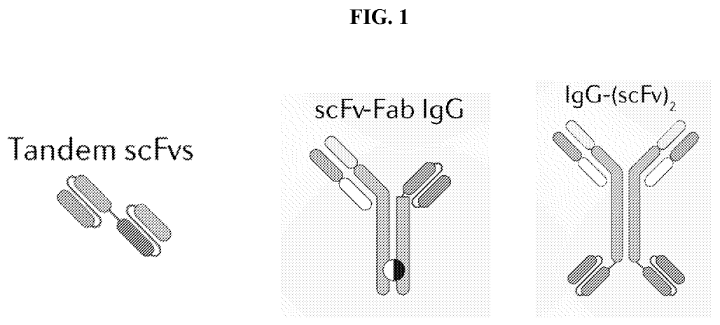

The experiments of this example demonstrate, inter alia, an antibody specific for claudin 6 and CD3. In this example, antibodies, antibody fragments, and antibody variants specific for claudin 6 and CD3 were isolated and characterized. This example identifies a tandem single-chain variable fragment (scFv) antibody, a scFv-Fab Fc antibody, an IgG-(scFV)₂ antibody, and a IgG-scFv Fc specific for claudin 6 and CD3 (FIG. 1).

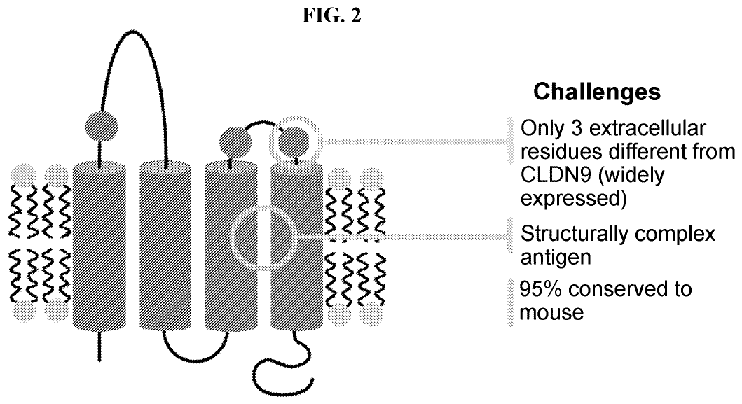

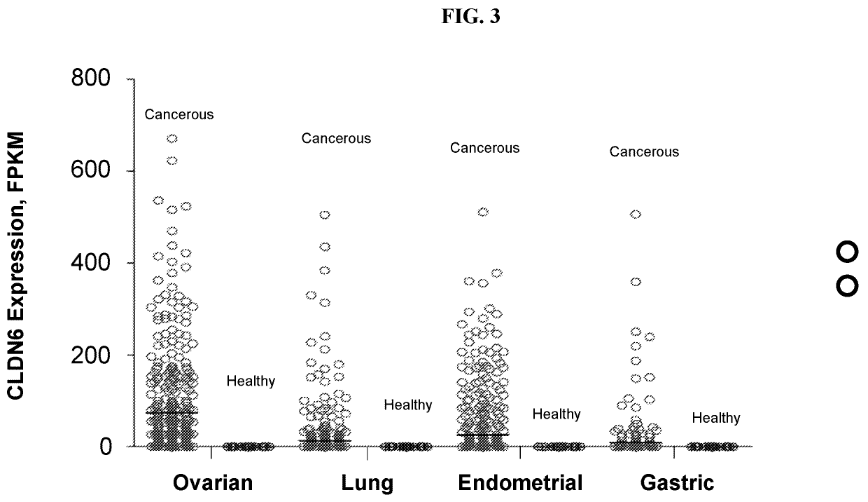

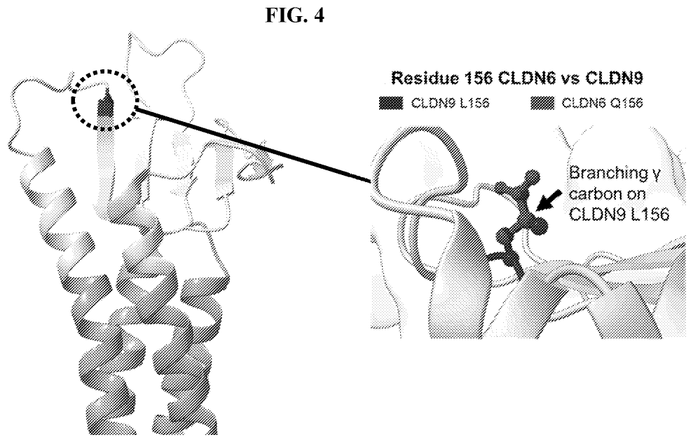

FIG. 2 is an image showing a schematic of the structure of CLDN6. Claudin 6 is expressed in ovarian, endometrial, and many other solid tumors, and based on the present disclosure, can be engaged by a bispecific antibody. Claudin 6 is not expressed in normal adult (mature) tissues. However, Claudin 6 is overexpressed in multiple cancers, including ovarian cancer, lung cancer, endometrial cancer, and gastric cancer (FIG. 3). In addition, FIG. 2 shows several of the illustrative challenges in developing an antibody specific for claudin 6. For example, Claudin 6 has only three extracellular residues that are different from CLDN9, which is widely expressed. Claudin 6 also is a structurally complex antigen, and is 95% conserved in mice. Further challenges in developing an antibody specific for claudin 6 include the “selectivity” challenge, as shown in FIG. 4. A selective CLDN6 antibody needs to discriminate between a single amino acid side chain at residue 156 on CLDN6 (Q156) and CLDN9 (L266). These challenges in the development of a selective CLDN6 antibody also hold true for the development of any bispecific construct that targets CLDN6, such as the CLDN6: CD3 bispecific constructs provided for herein.

FIG. 32 shows the epitope of the IM271-1HEP antibody, the CLDN6 binding arm of the IMC-16-3 scFv-Fab IgG antibody, as compared to the epitope of the benchmark IMAB027 antibody. Energetically critical epitope residues were determined by alanine scanning mutagenesis and flow cytometry. The left image in FIG. 32 shows the epitope of IM271-1HEP antibody while the right image in FIG. 32 shows the IMAB027 epitope.

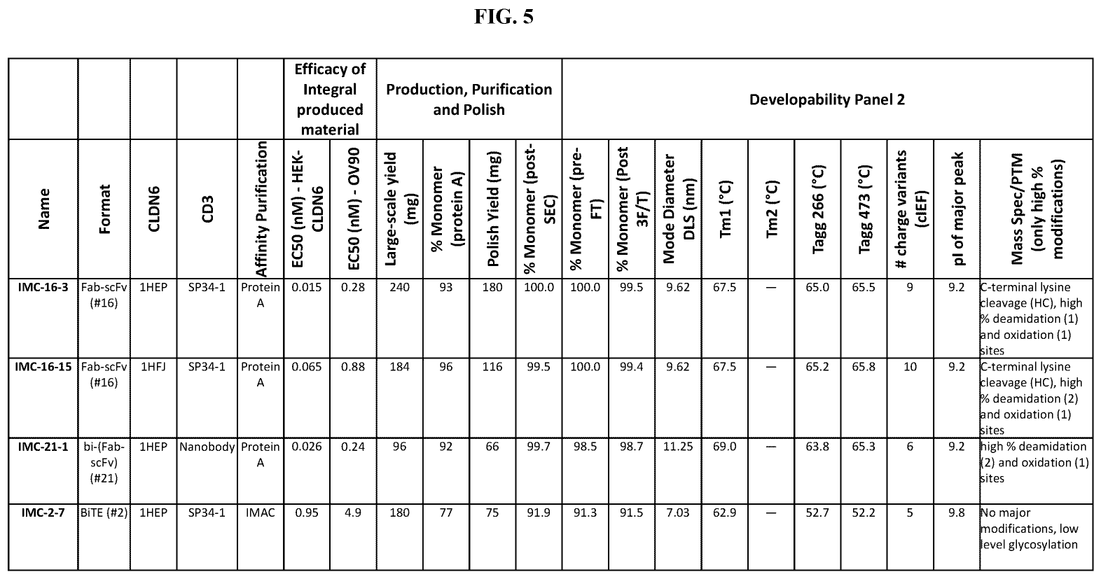

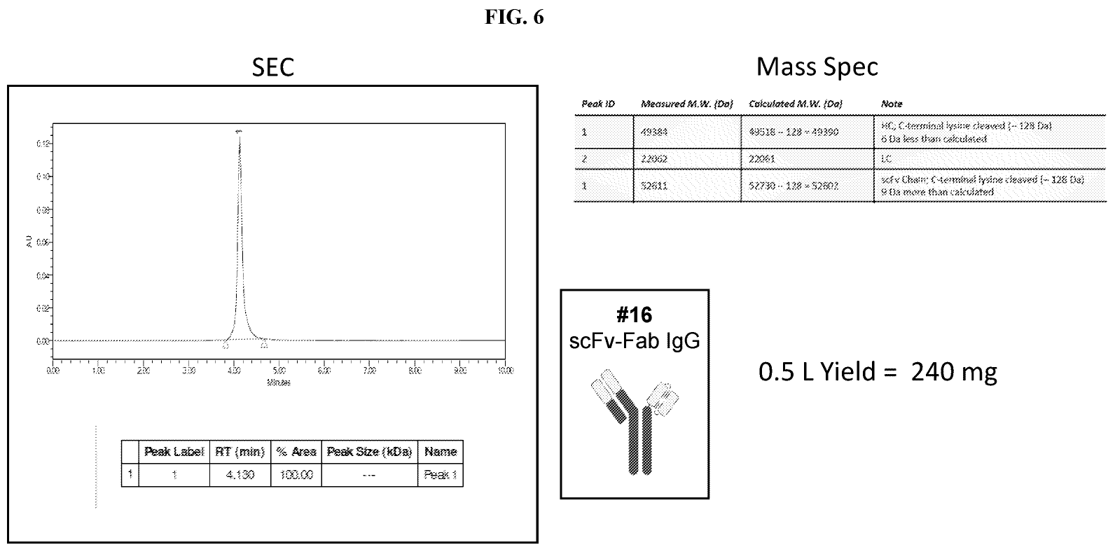

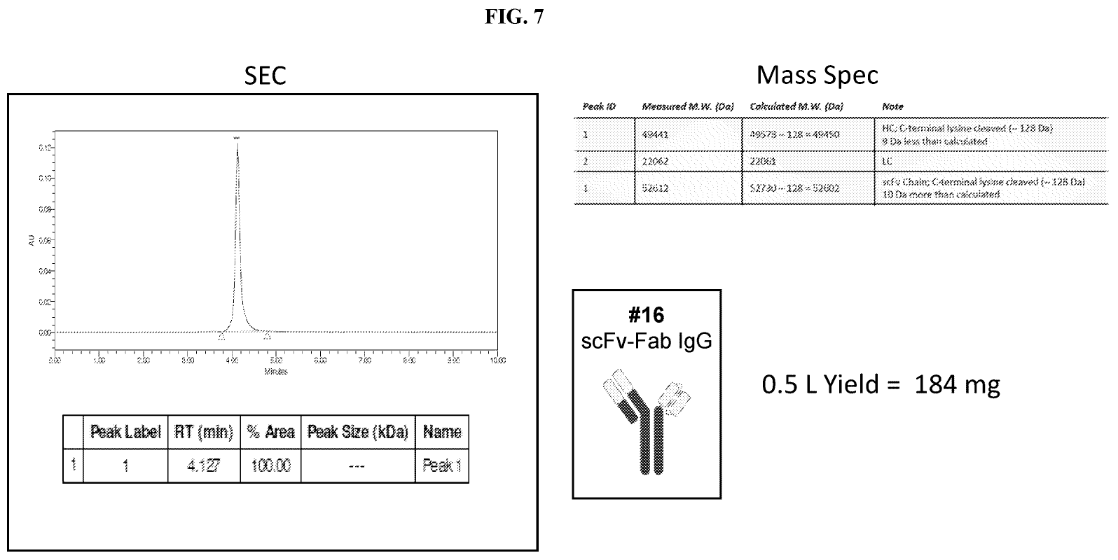

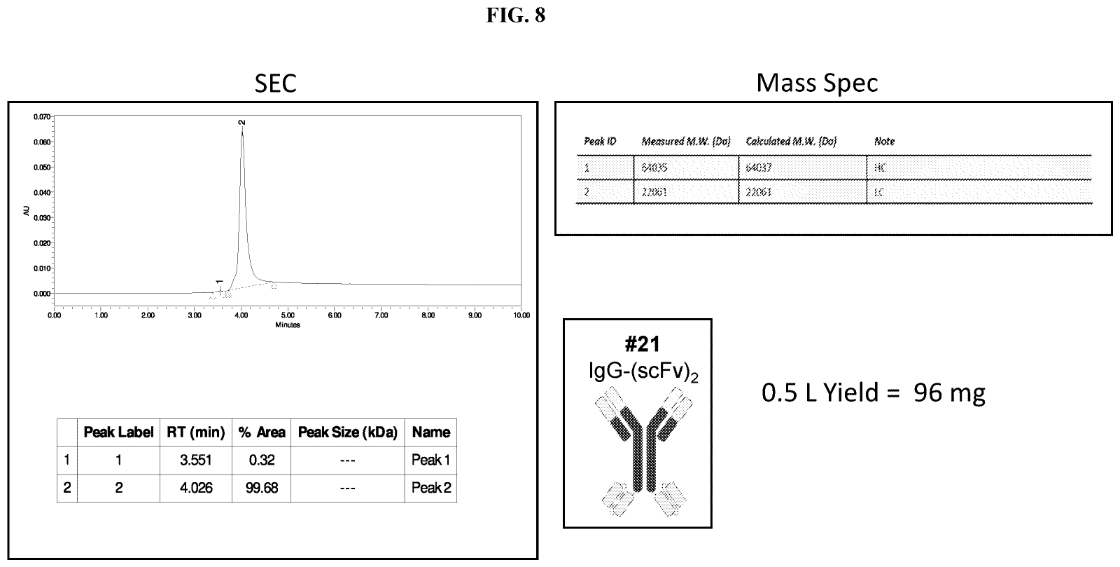

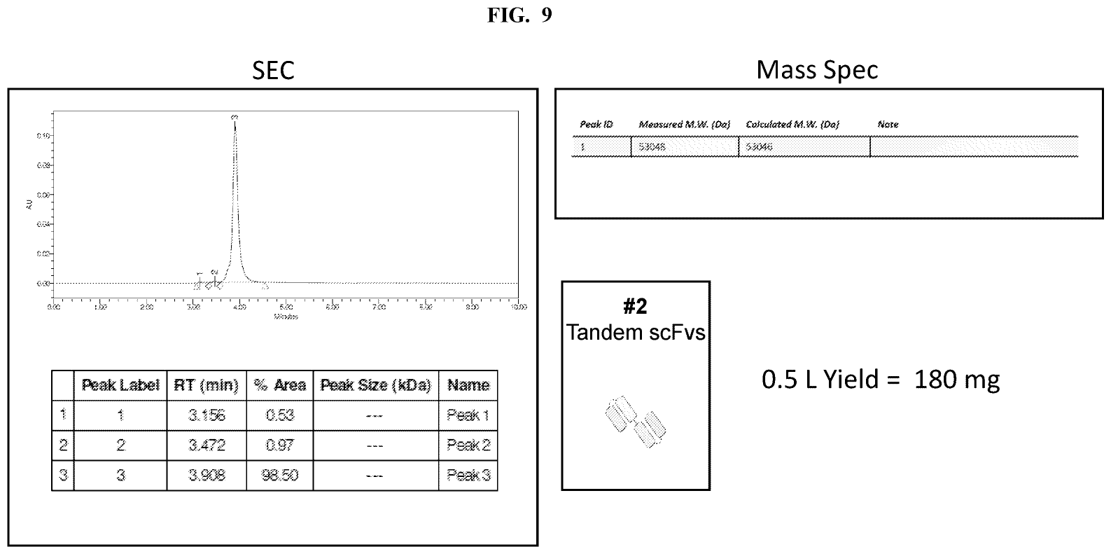

To develop and select a bispecific antibody to claudin 6 and CD3, a large panel of constructs were generated using the following antibody formats: tandem scFvs, scFv-Fab IgG, IgG-(scFv)₂, or IgG-scFv. Potential CLDN6 specific arms included those from HEP, HHP, HFJ, and 271 antibodies, and specific CD3 arms included those from muOKT3, huSP31-1, and huSP34-3 antibodies, as well as a nanobody. Based on selective binding to CLDN6 vs CLDN9, CLDN4, and CLDN3, experimental in vitro potency (including CLDN6+ cell killing and cytokine production), developability (including, without limitation, large-scale yield), and format/arm diversity, four molecules were selected for in vivo testing: an scFv-Fab IgG antibody (referred to as IMC-16-3; SEQ ID NOs: 79, 67, and 89), an scFv-Fab IgG antibody (referred to as IMC-16-15; SEQ ID NOs: 114, 65, and 89), an IgG-(scFv)₂ antibody (referred to as IMC-21-1; SEQ ID NOs: 86, and 67), and a tandem scFv antibody (referred to as IMC-2-7; SEQ ID NO: 95). FIG. 5, FIG. 6, FIG. 7, FIG. 8, and FIG. 9 show quality control data for the IMC-16-3 scFv-Fab IgG antibody (FIG. 6), IMC-16-15 scFv-Fab IgG antibody (FIG. 7), IMC-21-1 IgG-(scFv)₂ antibody (FIG. 8), and the IMC-2-7 tandem scFv antibody (FIG. 9) specific to human CLDN6 and CD3.

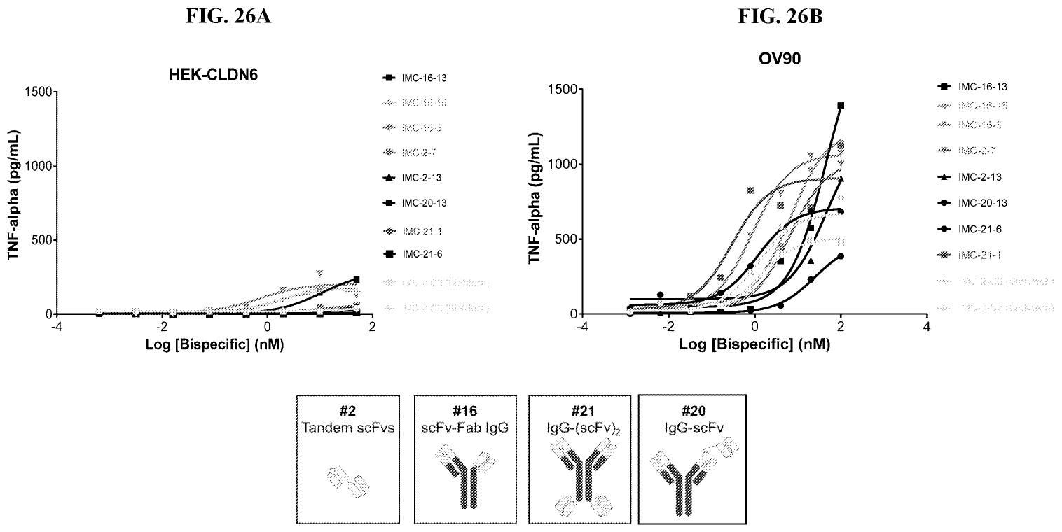

The experiments in FIG. 26A and FIG. 26B show TNF-α levels produced by human T cells in the presence of claudin 6 expressing HEK cells and OV-90 cells and a set of antibody constructs (i.e., IMC-16-13 scFv-Fab IgG antibody (SEQ ID NOs: 114, 65, and 80), IMC-16-15 scFv-Fab IgG antibody, IMC-16-3 scFv-Fab IgG antibody, IMC-2-7 tandem scFv antibody, IMC-2-13 tandem scFv antibody (SEQ ID NO: 115), IMC-20-13 IgG-scFv antibody (SEQ ID NOs: 114, 65, and 116), IMC-21-1 IgG-(scFv)2 antibody, IMC-21-6 IgG-(scFv)2 antibody (SEQ ID NOs: 104 and 66), and the IMC-2-3 tandem scFv antibody (SEQ ID NO: 84).

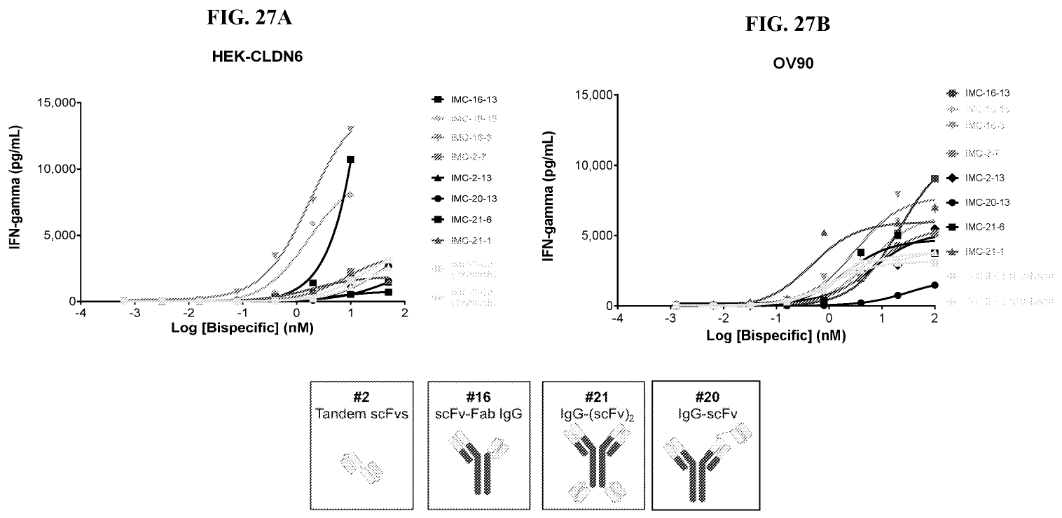

The experiments in FIG. 27A and FIG. 27B are graphs showing IFN-γ produced by human T cells in the presence of claudin 6 expressing HEK cells and OV-90 cells for a set of antibody constructs (i.e., IMC-16-13 scFv-Fab IgG antibody, IMC-16-15 scFv-Fab IgG antibody, IMC-16-3 scFv-Fab IgG antibody, IMC-2-7 tandem scFv antibody, IMC-2-13 tandem scFv antibody, IMC-20-13 IgG-scFv antibody, IMC-21-1 IgG-(scFv)2 antibody, IMC-21-6 IgG-(scFv)2 antibody, and the IMC-2-3 tandem scFv antibody).

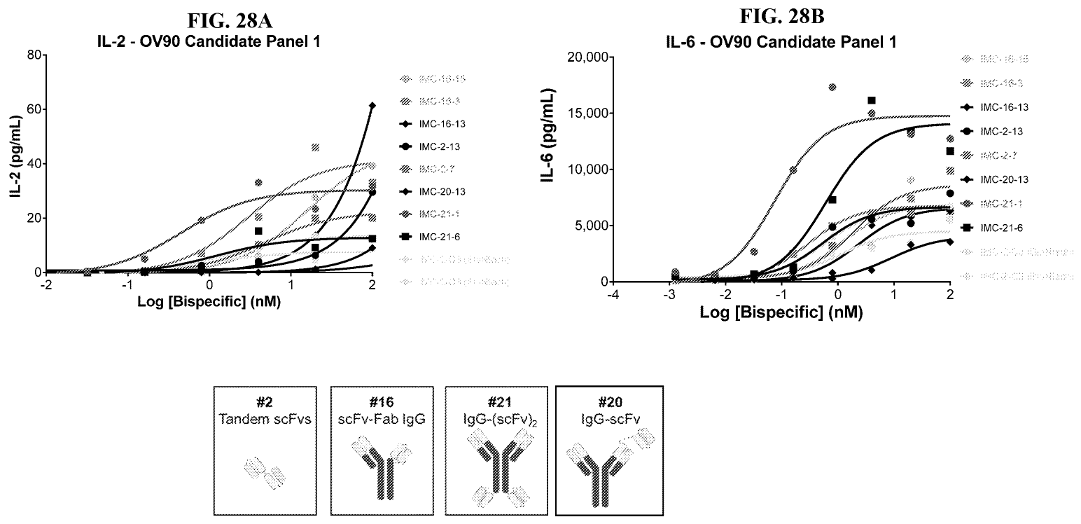

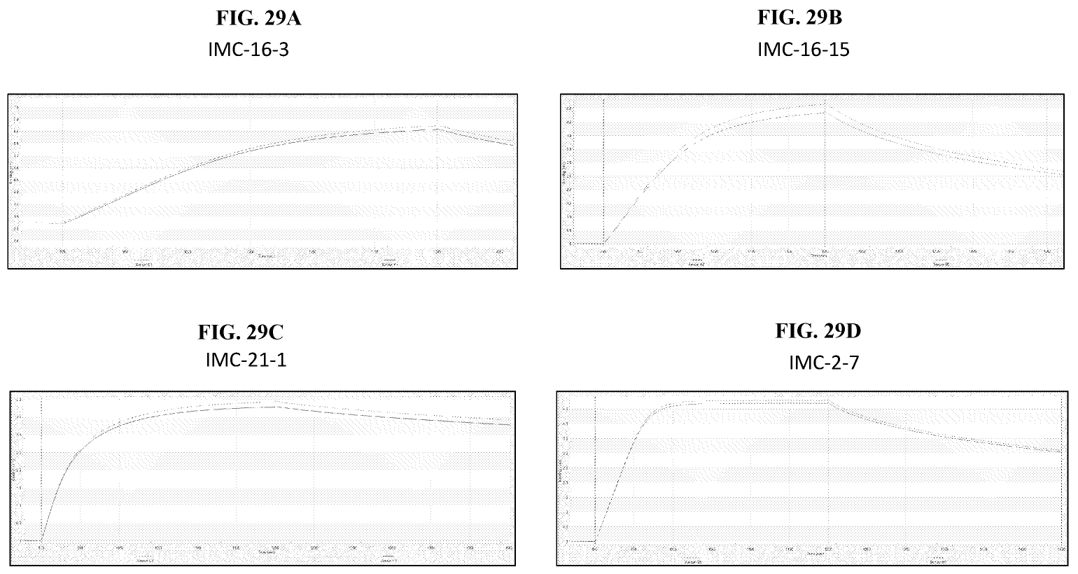

The experiments in FIG. 28A and FIG. 28B show IL-2 levels (FIG. 28A) and IL-6 levels (FIG. 28B) produced by human T cells in the presence of claudin 6 expressing OV-90 cells for a set of antibody constructs (i.e., IMC-16-13 scFv-Fab IgG antibody, IMC-16-15 scFv-Fab IgG antibody, IMC-16-3 scFv-Fab IgG antibody, IMC-2-7 tandem scFv antibody, IMC-2-13 tandem scFv antibody, IMC-20-13 IgG-scFv antibody, IMC-21-1 IgG-(scFv)2 antibody, IMC-21-6 IgG-(scFv)2 antibody, and the IMC-2-3 tandem scFv antibody). IL-6 and 11-2 levels were both marginal on the HEK-CLDN6 (<10 μg/ml).

The experiments of this example demonstrated that each selected antibody produced high yields. The IMC-16-3 scFv-Fab IgG antibody had the highest yield at 240 mg from a 0.5 L run. These experiments also demonstrated that minimal or no aggregates were observed for any of the molecules. The IMC-2-7 tandem scFv antibody had moderate levels of aggregation prior to post-polish using size-exclusion chromatography. The isoelectric points were also sufficiently high to allow wider range of pH for final buffer formulations.

### Example 2: Target Binding to Human CLDN6 and Human CD3

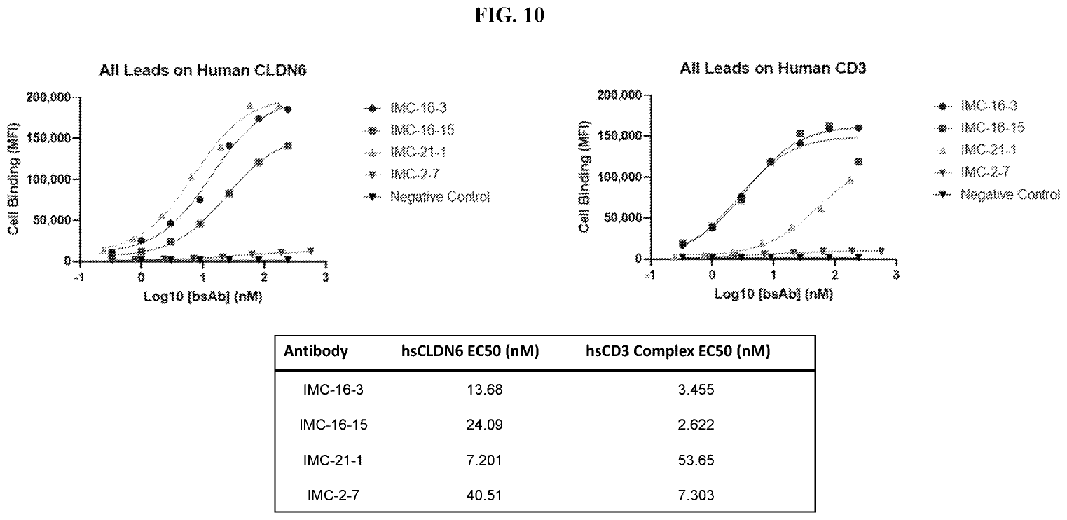

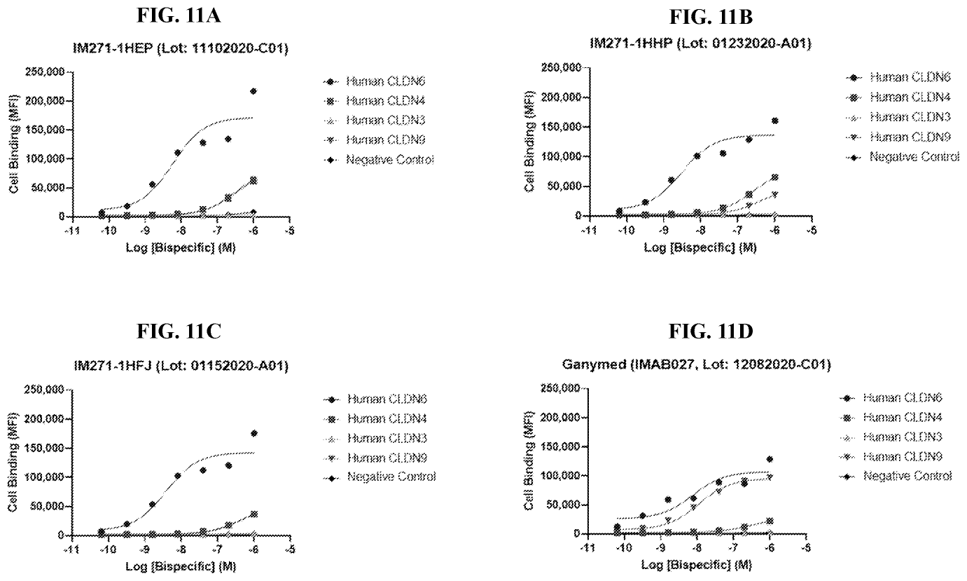

The experiments of this example demonstrated the antibodies disclosed herein bind to both human CLDN6 and human CD3. The experiments in FIG. 10 show two graphs of target binding data from flow cytometry experiments. The left panel in FIG. 10 shows target binding data of the IMC-16-3 scFv-Fab IgG antibody, IMC-16-15 scFv-Fab IgG antibody, IMC-21-1 IgG-(scFv)₂ antibody, and the IMC-2-7 tandem scFv antibody to human CLDN6-expressing cells. The right panel in FIG. 10 shows target binding data of the IMC-16-3 scFv-Fab IgG antibody, IMC-16-15 scFv-Fab IgG antibody, IMC-21-1 IgG-(scFv)₂ antibody, and the IMC-2-7 tandem scFv antibody to human CD3-expressing cells. These experiments show, inter alia, how the antibodies disclosed herein bind to both human CLDN6 and human CD3.

The experiments in FIG. 11A, FIG. 11B, FIG. 11C, and FIG. 11D show parental and benchmark antibody binding data from flow cytometry experiments. For each experiment, a CLDN negative parental cell line was either mock transfected or transfected with CLDN6, CLDN4, CLDN3, or CLDN9. FIG. 11A is a graph showing binding of IM271-1HEP antibody to human CLDN6 (circle shapes), human CLDN4 (square shapes), human CLDN3 (triangle shapes), human CLDN9 (inverted triangle shapes), and a negative control (diamond shapes). This experiment shows the IM271-1HEP antibody specifically binds to human CLDN6. FIG. 11B is a graph showing binding of IM271-1HHP antibody to human CLDN6 (circle shapes), human CLDN4 (square shapes), human CLDN3 (triangle shapes), human CLDN9 (inverted triangle shapes), and a negative control (diamond shapes). This experiment shows the IM271-1HHP antibody specifically binds to human CLDN6. FIG. 11C is a graph showing binding of IM271-1HFJ antibody to human CLDN6 (circle shapes), human CLDN4 (square shapes), human CLDN3 (triangle shapes), human CLDN9 (inverted triangle shapes), and a negative control (diamond shapes). This experiment shows the IM271-1HFJ antibody specifically binds to human CLDN6. FIG. 11D is a graph showing binding of an antibody to human CLDN6 (circle shapes), human CLDN4 (square shapes), human CLDN3 (triangle shapes), human CLDN9 (inverted triangle shapes), and a negative control (diamond shapes). This experiment shows how antibody in FIG. 11D does not specifically bind to human CLDN6, but binds to CLDN6 and CLDN9 equally. Collectively, these experiments show, inter alia, how CLDN6 specific arms from HEP, HHP, HFJ, and 271 antibodies bind specifically to human CLDN6.

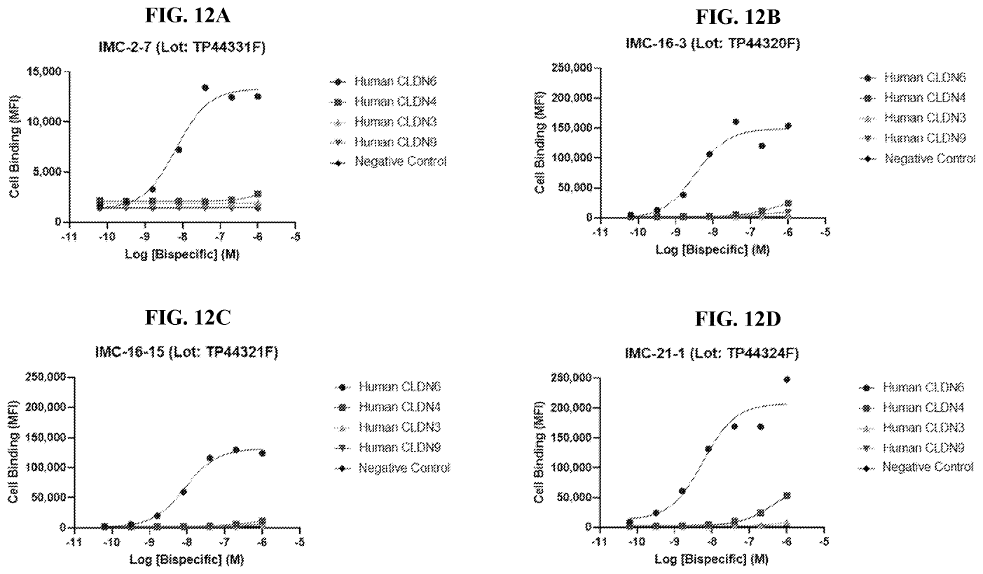

The experiments in FIG. 12A, FIG. 12B, FIG. 12C, and FIG. 12D are graphs showing target binding data from flow cytometry experiments. For each experiment, a CLDN negative parental cell line was either mock transfected or transfected with CLDN6, CLDN4, CLDN3, or CLDN9. Each experiment demonstrates that selected antibodies have one arm that specifically binds to human CLDN6. FIG. 12A is a graph showing binding of the IMC-2-7 tandem scFv antibody to human CLDN6 (circle shapes), human CLDN4 (square shapes), human CLDN3 (triangle shapes), human CLDN9 (inverted triangle shapes), and a negative control (diamond shapes). This experiment shows that the IMC-2-7 tandem scFv antibody specifically binds to human CLDN6. FIG. 12B is a graph showing binding of the IMC-16-3 scFv-Fab IgG antibody to human CLDN6 (circle shapes), human CLDN4 (square shapes), human CLDN3 (triangle shapes), human CLDN9 (inverted triangle shapes), and a negative control (diamond shapes). This experiment shows that the IMC-16-3 scFv-Fab IgG antibody specifically binds to human CLDN6. FIG. 12C is a graph showing binding of the IMC-16-15 scFv-Fab IgG antibody to human CLDN6 (circle shapes), human CLDN4 (square shapes), human CLDN3 (triangle shapes), human CLDN9 (inverted triangle shapes), and a negative control (diamond shapes). This experiment shows that the IMC-16-15 scFv-Fab IgG antibody specifically binds to human CLDN6. FIG. 12D is a graph showing binding of the IMC-21-1 IgG-(scFv)₂ antibody to human CLDN6 (circle shapes), human CLDN4 (square shapes), human CLDN3 (triangle shapes), human CLDN9 (inverted triangle shapes), and a negative control (diamond shapes). This experiment shows, inter alia, that the IMC-21-1 IgG-(scFv)₂ antibody specifically binds to human CLDN6.

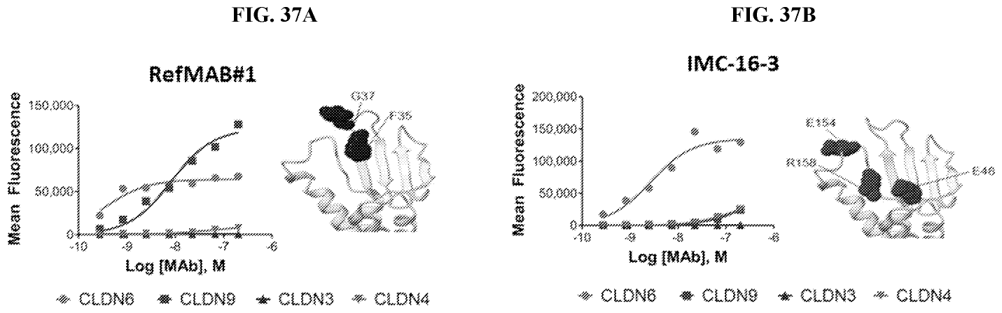

The experiment in FIG. 37 shows target binding data from flow cytometry experiments utilizing a reference antibody (RefMAB #1) and IMC-16-3. For this experiment, a CLDN negative parental cell line was transfected with CLDN6, CLDN4, CLDN3, or CLDN9. The data show binding of the antibodies to human CLDN6 (circle shapes), human CLDN4 (square shapes), human CLDN3 (triangle shapes), human CLDN9 (inverted triangle shapes), and a negative control (diamond shapes). Epitope binding data are also shown for each antibody. These data highlight that the RefMAB #1, which binds to a different epitope than IMC-16-3, shows significant binding to CLDN9, while IMC-16-3 is highly selective for CLDN6.

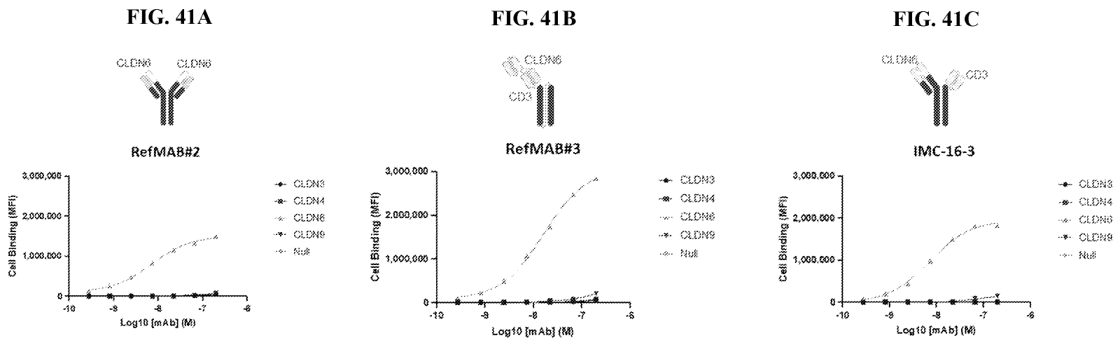

The experiment in FIG. 41 shows comparative target binding data between an anti-CLDN6 antibody-drug conjugate (ACD) (RefMAB #2; FIG. 41A), an anti-CLDN6× anti-CD3 BiTE molecule (RefMAB #3; FIG. 41B) and IMC-16-3 (FIG. 41C). The ADC comprises an anti-CLDN6 antibody conjugated to monomethyl auristatin E (MMAE). Binding assessment was performed after HEK293F cells were transfected with nucleic acid molecules to express various claudin proteins. These comparative data show binding of the antibodies to human CLDN3 (circle shapes), human CLDN4 (square shapes), human CLDN6 (triangle shapes), human CLDN9 (inverted triangle shapes), and a negative control (diamond shapes). Each of RefMAB #2, RefMAB #3, and IMC-16-3 were highly selective for CLDN6 over the other claudin molecules assayed, in contrast to molecules typified by RefMAB #1 (FIG. 37) which was not selective for CLDN6.

The experiments of this example demonstrate, inter alia, that the antibodies disclosed herein bind to both human CLDN6 and human CD3. The antibodies disclosed herein show a high degree of specificity in binding to CLDN6 over CLDNs 3, 4, or 9.

### Example 3: T-Cell Mediated Cytotoxicit Assays

The experiments of this example demonstrated, inter alia, that the antibodies disclosed herein have high levels of T cell mediated killing specific in both endogenous and exogenous models of CLDN6 expression (OV-90 cells and CLDN6-K562 cells, respectively).

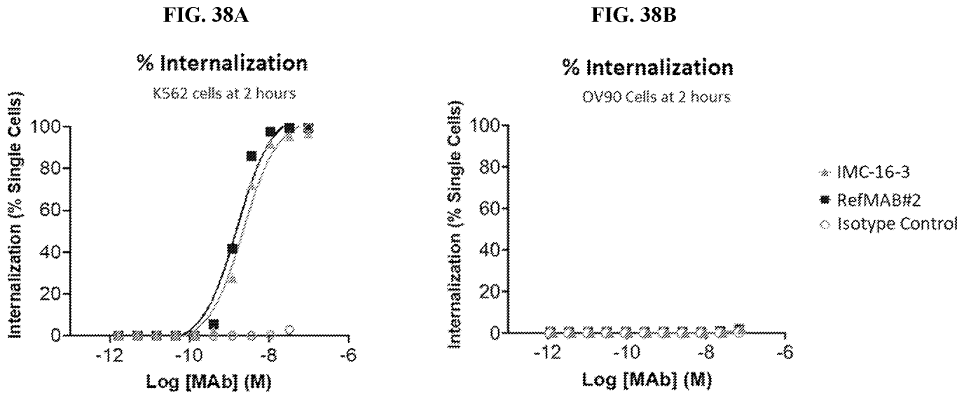

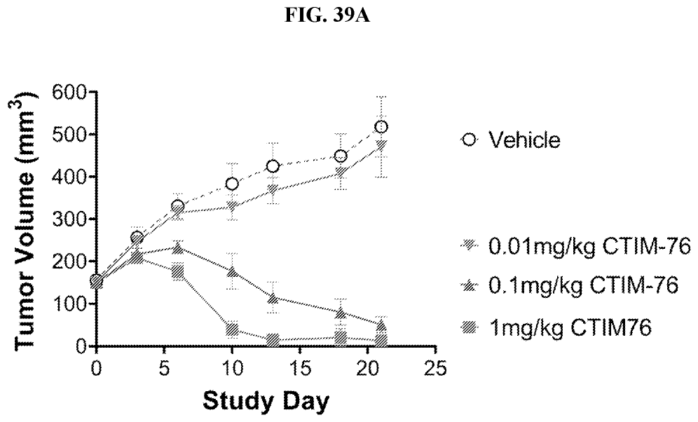

The experiments in FIG. 38 illustrate the results of an internalization study to determine whether IMC-16-3 or a reference antibody (RefMAB #2) become internalized after CLDN6 binding. IMC-16-3 and the reference antibody were labeled with the pH-sensitive dye pHrodo and incubated with either K562 cells, which stably overexpress high levels of CLDN6, or OV-90 ovarian cancer cells, which express moderate levels of CLDN6, for 2 hours. As shown in FIG. 38, both antibodies are only internalized in cells which express high levels of CLDN6. These data indicate that antibodies targeting CLDN6 are slowly internalized in moderate to low CLDN6 expressing cells, which makes CLDN6 an ideal target for a T cell engaging bispecific antibody.

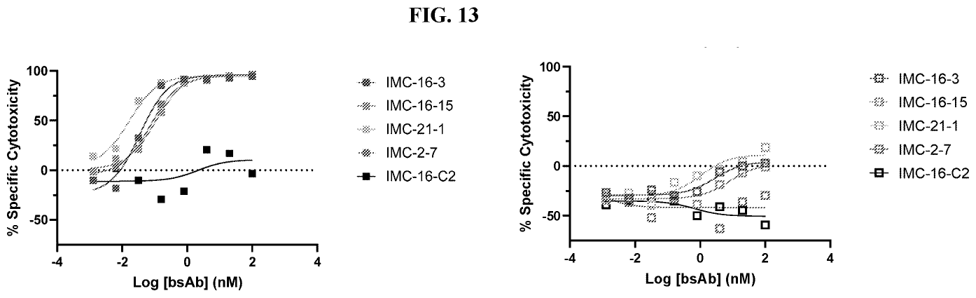

The experiments in FIG. 13 show two graphs of cellular toxicity data from T-cell dependent cellular cytotoxicity experiments. The left panel in FIG. 13 shows cytotoxicity data of human T cells co-cultured with the IMC-16-3 scFv-Fab IgG antibody, IMC-16-15 scFv-Fab IgG antibody, IMC-21-1 IgG-(scFv)₂ antibody, IMC-2-7 tandem scFv antibody, and the IMC-16-C2 antibody (negative control) and ovarian cancer OV-90 cells, which express CLDN6. The right panel in FIG. 13 shows cytotoxicity data of human T cells co-cultured with the IMC-16-3 scFv-Fab IgG antibody, IMC-16-15 scFv-Fab IgG antibody, IMC-21-1 IgG-(scFv)₂ antibody, IMC-2-7 tandem scFv antibody, and the IMC-16-C2 antibody and HEK cells. These experiments show, inter alia, that killing was only potently induced versus CLDN6 expressing OV-90 cells.

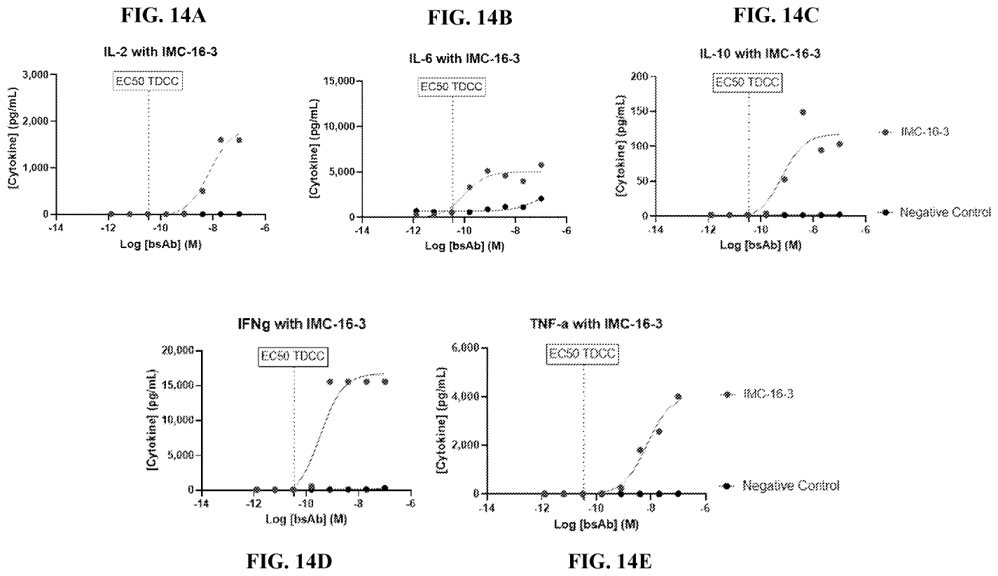

The experiments in FIG. 14A, FIG. 14B, FIG. 14C, FIG. 14D, and FIG. 14E are graphs showing cytokines produced by human PBMC cultured with the IMC-16-3 scFv-Fab IgG antibody and OV-90 cells as compared to cellular toxicity data. FIG. 14A shows the levels of IL-2, FIG. 14B shows the levels of IL-6, FIG. 14C shows the levels of IL-10, FIG. 14D shows the levels of IFN-7, and FIG. 14E shows the levels of TNF-α produced by human PBMC in the presence of OV-90 cells and IMC-16-3. These experiments show, inter alia, that IL-2, IL-6, IL-10, IFN-7, and TNF-α were all induced in human PBMC by the IMC-16-3 scFv-Fab IgG antibody in the presence of CLDN6-expressing OV-90 cells.

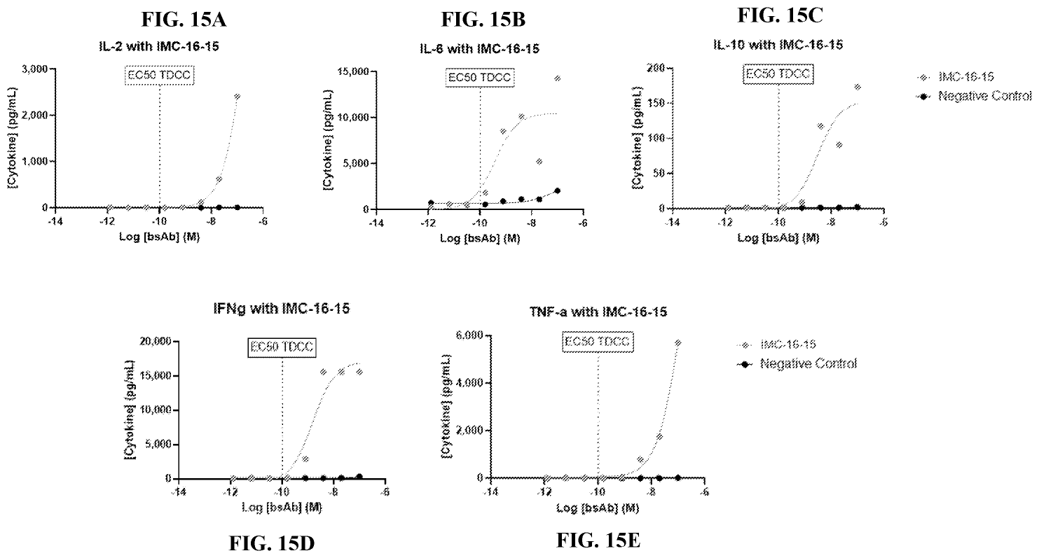

The experiments in FIG. 15A, FIG. 15B, FIG. 15C, FIG. 15D, and FIG. 15E are graphs showing cytokines produced by human PBMC cultured with the IMC-16-15 scFv-Fab IgG antibody and OV-90 cells. FIG. 15A shows the levels of IL-2, FIG. 15B shows the levels of IL-6, FIG. 15C shows the levels of IL-10, FIG. 15D shows the levels of IFN-7, and FIG. 15E shows the levels of TNF-α produced by human PBMC in the presence of OV-90 cells and IMC-16-15. These experiments show, inter alia, that IL-2, IL-6, IL-10, IFN-7, and TNF-α were all induced in human PBMC by the IMC-16-15 scFv-Fab IgG antibody in the presence of CLDN6-expressing OV-90 cells.

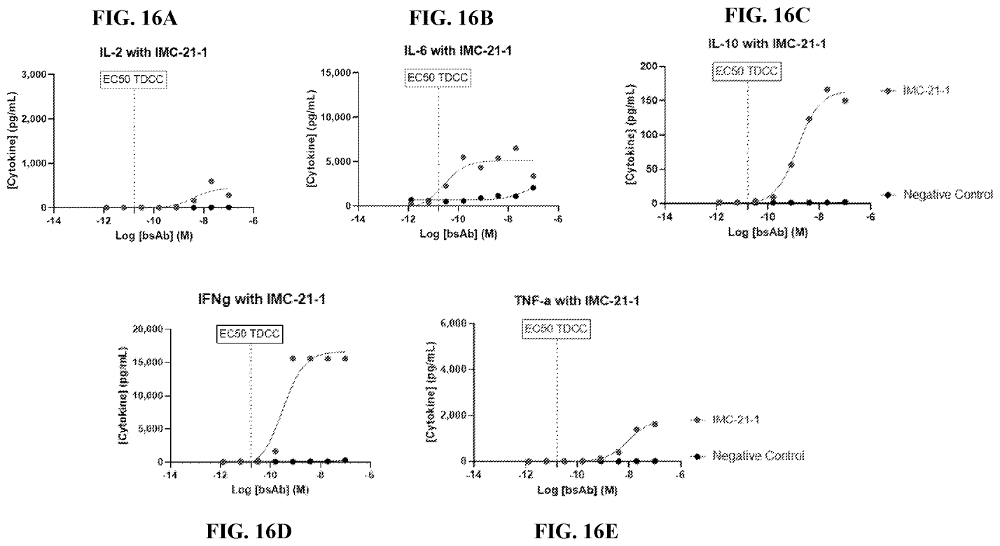

The experiments in FIG. 16A, FIG. 16B, FIG. 16C, FIG. 16D, and FIG. 16E are graphs showing cytokines produced by human PBMC cultured with the IMC-21-1 IgG-(scFv)₂ antibody in the presence of OV-90 cells. FIG. 16A shows the levels of IL-2, FIG. 16B shows the levels of IL-6, FIG. 16C shows the levels of IL-10, FIG. 16D shows the levels of IFN-7, and FIG. 16E shows the levels of TNF-α produced by human PBMC in the presence of OV-90 cells and IMC-21-1. These experiments show, inter alia, that IL-2, IL-6, IL-10, IFN-7, and TNF-α were all induced in human PBMC by the IMC-21-1 IgG-(scFv)₂ antibody in the presence of CLDN6-expressing OV-90 cells.

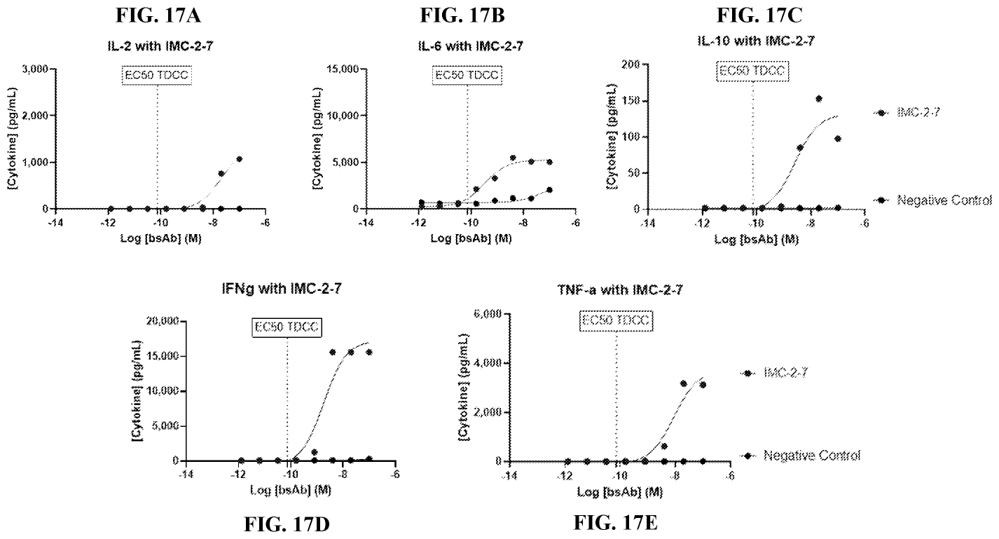

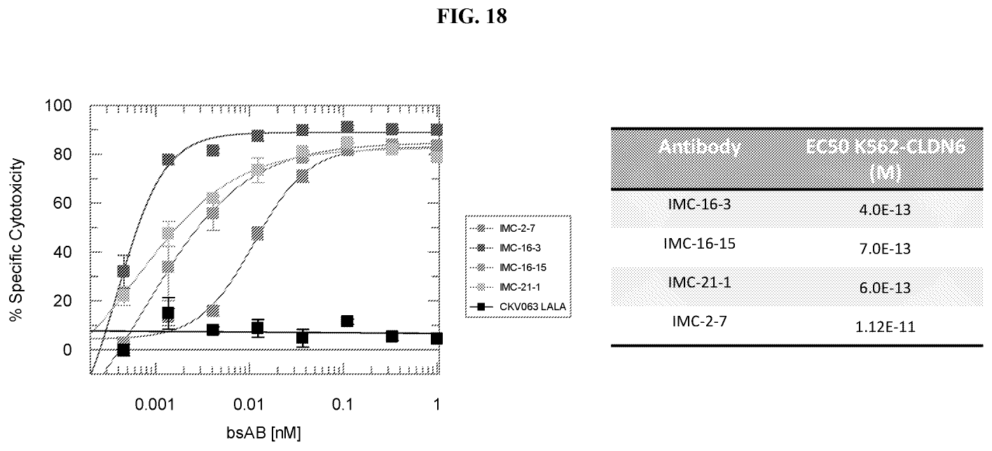

The experiments in FIG. 17A, FIG. 17B, FIG. 17C, FIG. 17D, and FIG. 17E are graphs showing cytokines produced by human PBMC cultured with the IMC-2-7 tandem scFv antibody in the presence of OV-90 cells. FIG. 17A shows the levels of IL-2, FIG. 17B shows the levels of IL-6, FIG. 17C shows the levels of IL-10, FIG. 17D shows the levels of IFN-7, and FIG. 17E shows the levels of TNF-α produced by human PBMC in the presence of OV-90 cells and IMC-2-7. These experiments show, inter alia, that IL-2, IL-6, IL-10, IFN-7, and TNF-α were all induced in human PBMC by the IMC-2-7 tandem scFv antibody in the presence of CLDN-6 expressing OV-90 cells.

The experiments in FIG. 18 show a graph of cytotoxicity data from T-cell dependent cellular cytotoxicity experiments. FIG. 18 shows cytotoxicity data of human T cells cultured with the IMC-2-7 tandem scFv antibody, IMC-16-3 scFv-Fab IgG antibody, IMC-16-15 scFv-Fab IgG antibody, IMC-21-1 IgG-(scFv)₂ antibody, and a negative control (CKV063 LALA) versus CLDN6-expressing K562 cells. These experiments show, inter alia, that killing was potently induced in CLDN6 expressing K562 cells.

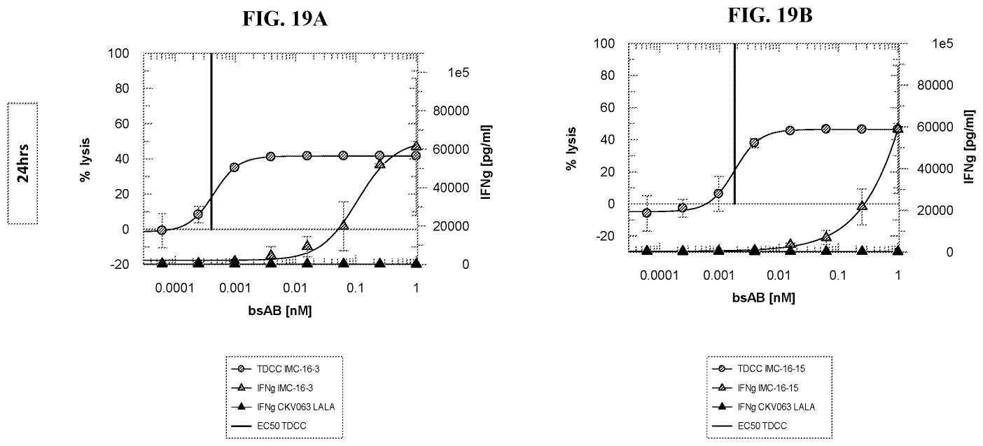

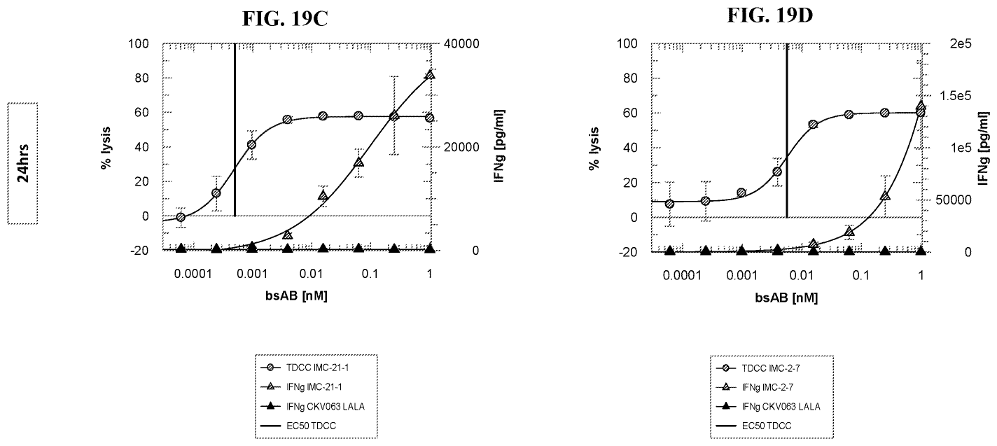

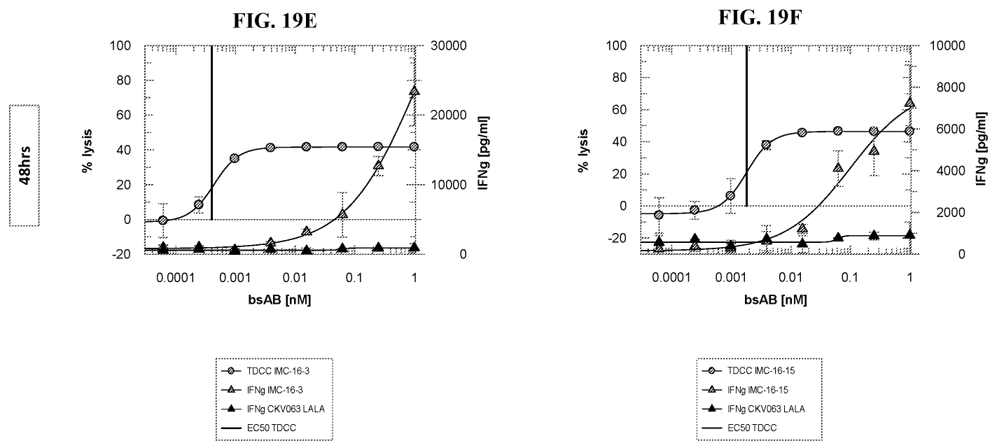

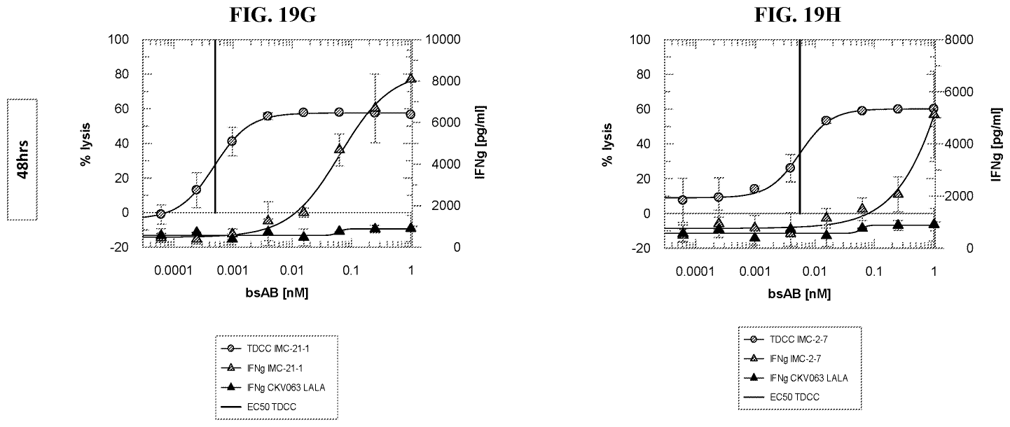

The experiments in FIG. 19A, FIG. 19B, FIG. 19C, FIG. 19D, FIG. 19E, FIG. 19F, FIG. 19G, and FIG. 19H demonstrate cellular toxicity and IFN-γ release potency data for human T cells cultured with CLDN6-expressing target cells at 24 hours and 48 hours from cellular cytotoxicity experiments. FIG. 19A shows cellular toxicity and IFN-γ release data of the IMC-16-3 scFv-Fab IgG antibody at 24 hours. FIG. 19B shows cellular toxicity and IFN-7 release data of the IMC-16-15 scFv-Fab IgG antibody at 24 hours. FIG. 19C shows cellular toxicity and IFN-γ release data of the IMC-21-1 IgG-(scFv)₂ antibody at 24 hours. FIG. 19D shows cellular toxicity and IFN-γ release data of the IMC-2-7 tandem scFv antibody at 24 hours. FIG. 19E shows cellular toxicity and IFN-γ release data of the IMC-16-3 scFv-Fab IgG antibody at 48 hours. FIG. 19F shows cellular toxicity and IFN-γ release data of the IMC-16-15 scFv-Fab IgG antibody at 48 hours. FIG. 19G shows cellular toxicity and IFN-γ release data of the IMC-21-1 IgG-(scFv)₂ antibody at 48 hours. FIG. 19H shows cellular toxicity and IFN-γ release data of the IMC-2-7 tandem scFv antibody at 48 hours. The CKV063 LALA antibody is a negative control antibody in each experiment. These experiments show, inter alia, T cell dependent cellular cytotoxicity and IFN-γ release potency at 24 hours and 48 hours for the antibodies described in this experiment.

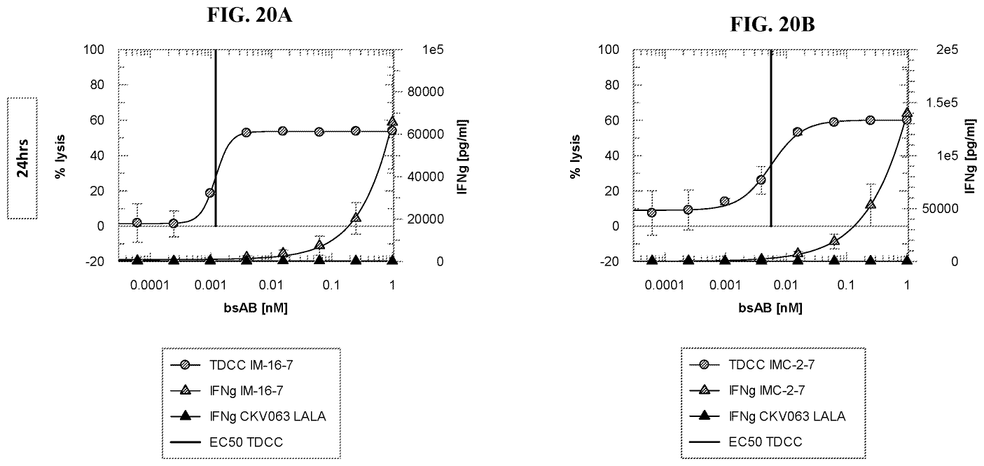

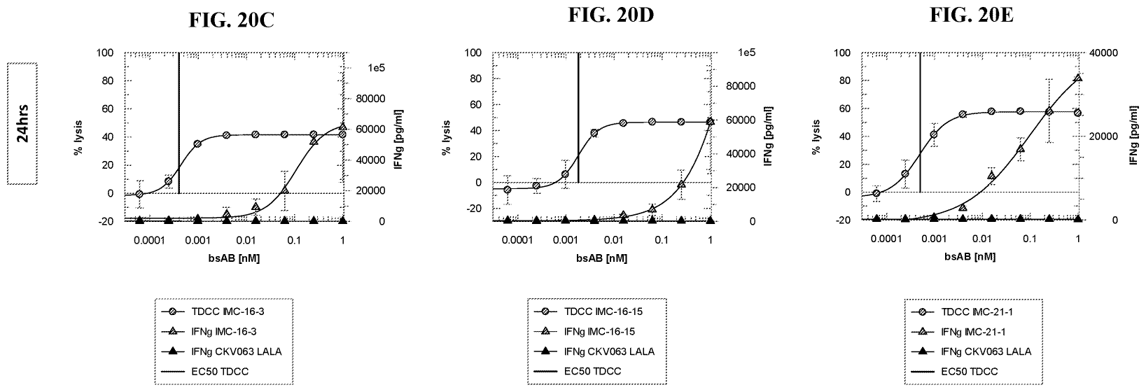

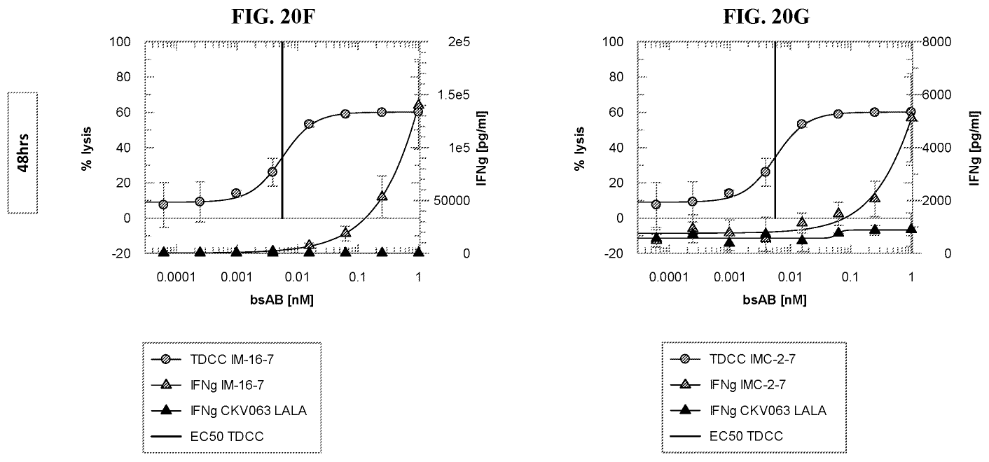

The experiments FIG. 20A, FIG. 20B, FIG. 20C, FIG. 20D, FIG. 20E, FIG. 20F, FIG. 20G, FIG. 20H, FIG. 20I, and FIG. 20J demonstrate cellular toxicity and IFN-γ release potency data for human T cells cultured with CLDN6-expressing target cells at 24 hours and 48 hours from cellular cytotoxicity experiments. FIG. 20A shows cellular toxicity and IFN-γ release data of the IMC-16-7 antibody (SEQ ID NOs: 79, 67, and 91) at 24 hours. FIG. 20B shows cellular toxicity and IFN-γ release data of the IMC-2-7 tandem scFv antibody at 24 hours. FIG. 20C shows cellular toxicity and IFN-γ release data of the IMC-16-3 scFv-Fab IgG antibody at 24 hours. FIG. 20D shows cellular toxicity and IFN-γ release data of the IMC-16-15 scFv-Fab IgG antibody at 24 hours. FIG. 20E shows cellular toxicity and IFN-γ release data of the IMC-21-1 IgG-(scFv)₂ antibody at 24 hours. FIG. 20F shows cellular toxicity and IFN-γ release data of the IMC-16-7 antibody at 48 hours. FIG. 20G shows cellular toxicity and IFN-γ release data of the IMC-2-7 tandem scFv antibody at 48 hours. FIG. 20H shows cellular toxicity and IFN-γ release data of the IMC-16-3 scFv-Fab IgG antibody at 48 hours. FIG. 20I shows cellular toxicity and IFN-γ release data of the IMC-16-15 scFv-Fab IgG antibody at 48 hours. FIG. 20J shows cellular toxicity and IFN-γ release data of the IMC-21-1 IgG-(scFv)₂ antibody at 48 hours. The CKV063 LALA antibody is a negative control antibody in each experiment. These experiments show, inter alia, T cell dependent cellular cytotoxicity and IFN-γ release potency at 24 hours and 48 hours for the antibodies described in this experiment.

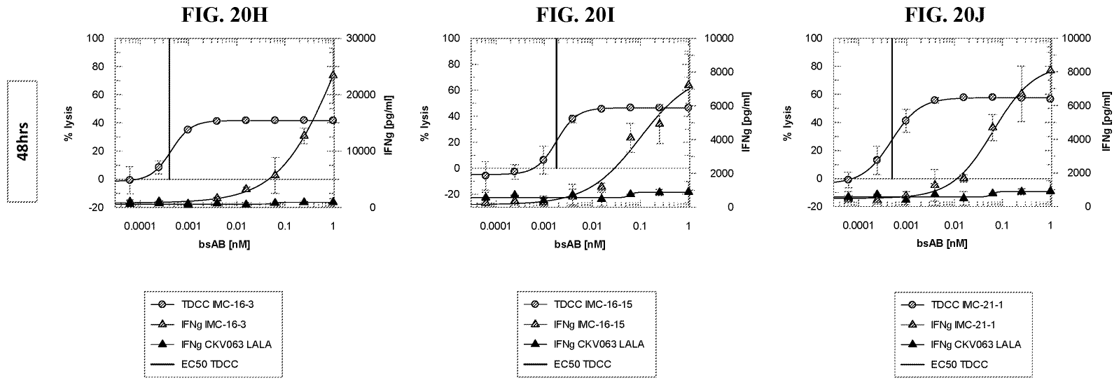

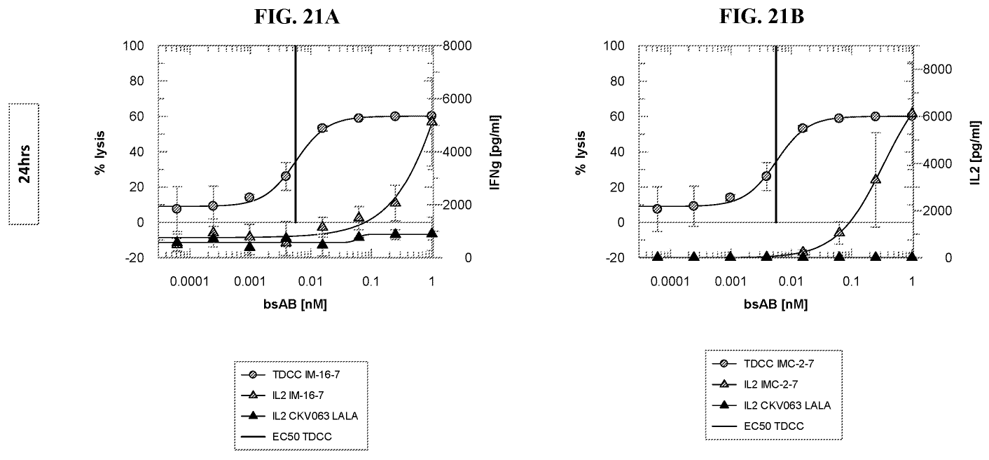

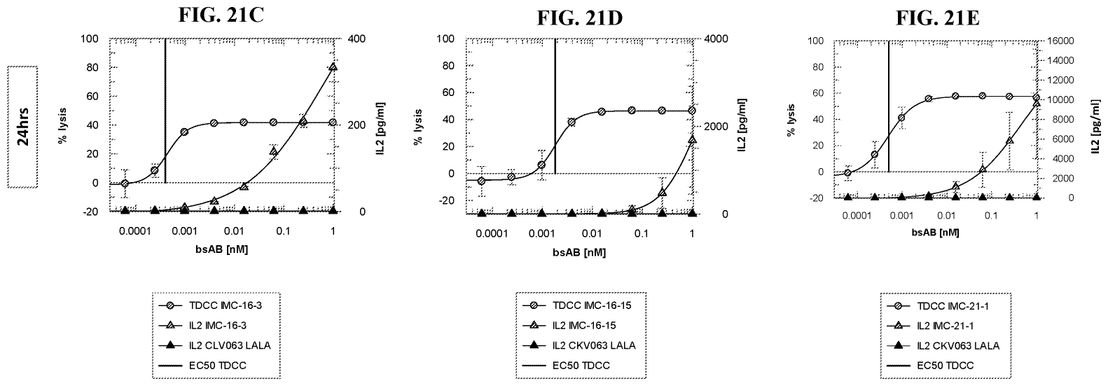

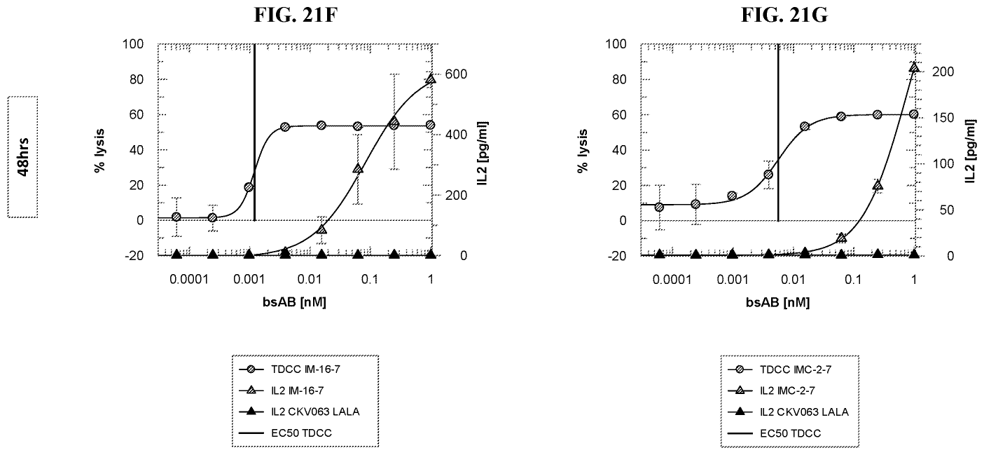

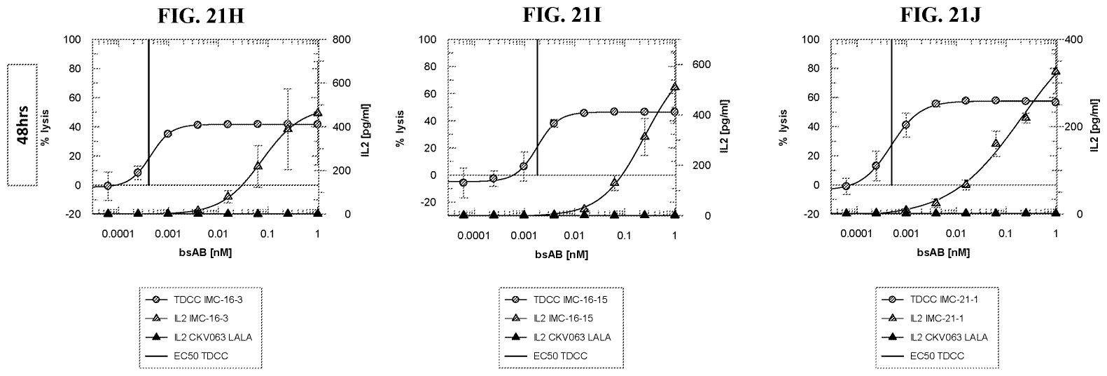

The experiments in FIG. 21A, FIG. 21B, FIG. 21C, FIG. 21D, FIG. 21E, FIG. 21F, FIG. 21G, FIG. 21H, FIG. 21I, and FIG. 21J demonstrate cellular toxicity and IL-2 release potency data at 24 hours and 48 hours from cellular cytotoxicity experiments. FIG. 21A shows cellular toxicity and IL-2 release data of the IMC-16-7 antibody at 24 hours. FIG. 21B shows cellular toxicity and IL-2 release data of the IMC-2-7 tandem scFv antibody at 24 hours. FIG. 21C shows cellular toxicity and IL-2 release data of the IMC-16-3 scFv-Fab IgG antibody at 24 hours. FIG. 21D shows cellular toxicity and IL-2 release data of the IMC-16-15 scFv-Fab IgG antibody at 24 hours. FIG. 21E shows cellular toxicity and IL-2 release data of the IMC-21-1 antibody at 24 hours. FIG. 21F shows cellular toxicity and IL-2 release data of the IMC-16-7 antibody at 48 hours. FIG. 21G shows cellular toxicity and IL-2 release data of the IMC-2-7 tandem scFv antibody at 48 hours. FIG. 21H shows cellular toxicity and IL-2 release data of the IMC-16-3 scFv-Fab IgG antibody at 48 hours. FIG. 21I shows cellular toxicity and IL-2 release data of the IMC-16-15 scFv-Fab IgG antibody at 48 hours. FIG. 21J shows cellular toxicity and IL-2 release data of the IMC-21-1 IgG-(scFv)₂ antibody at 48 hours. The CKV063 LALA antibody is a negative control antibody in each experiment. These experiments show, inter alia, T cell dependent cellular cytotoxicity and IL-12 release potency at 24 hours and 48 hours for the antibodies described in this experiment.

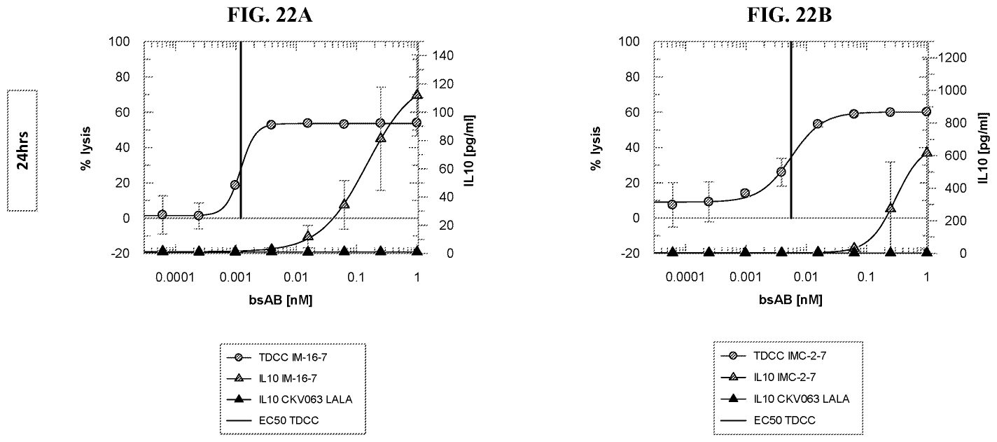

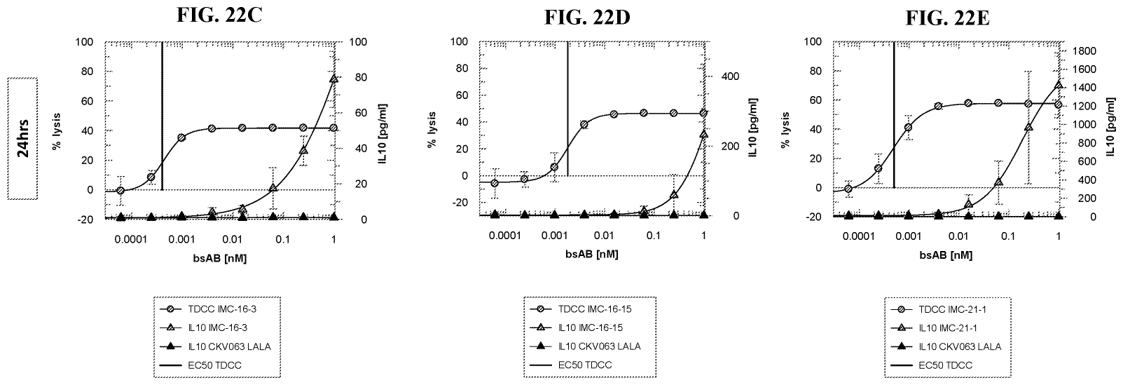

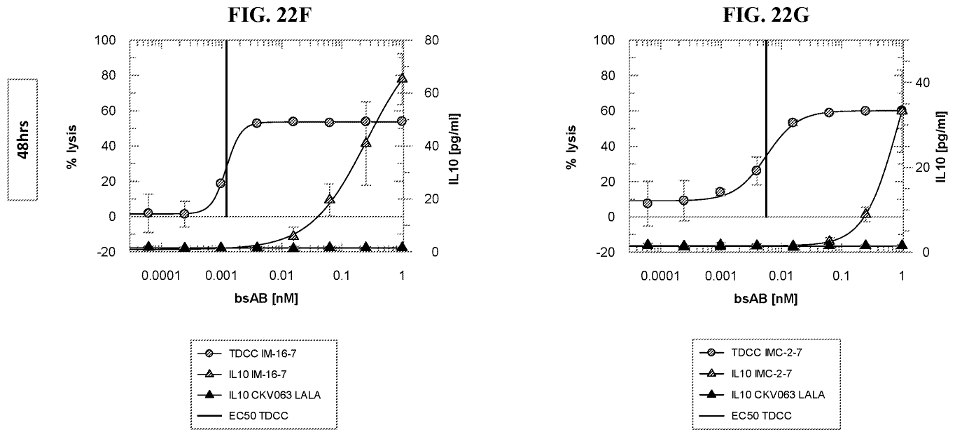

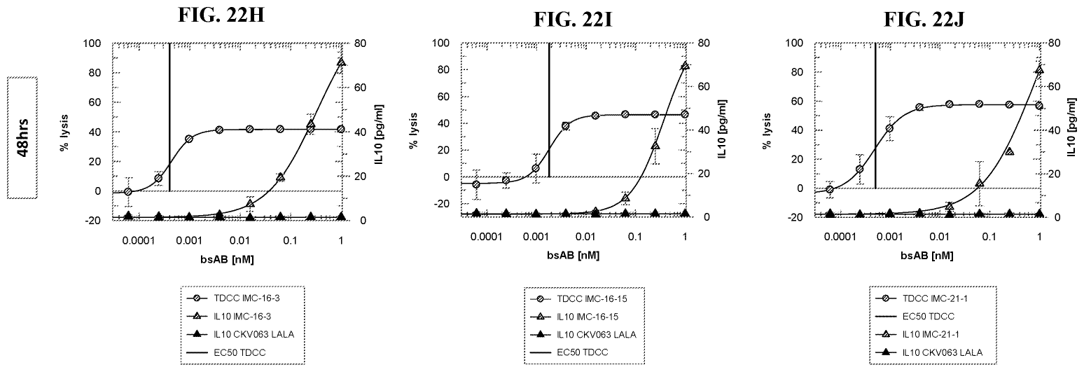

The experiments in FIG. 22A, FIG. 22B, FIG. 22C, FIG. 22D, FIG. 22E, FIG. 22F, FIG. 22G, FIG. 22H, FIG. 22I, and FIG. 22J demonstrate cellular toxicity and IL-10 release potency data at 24 hours and 48 hours from cytotoxicity experiments. FIG. 22A shows cellular toxicity and IL-10 release data of the IMC-16-7 antibody at 24 hours. FIG. 22B shows cellular toxicity and IL-10 release data of the IMC-2-7 tandem scFv antibody at 24 hours. FIG. 22C shows cellular toxicity and IL-10 release data of the IMC-16-3 scFv-Fab IgG antibody at 24 hours. FIG. 22D shows cellular toxicity and IL-10 release data of the IMC-16-15 scFv-Fab IgG antibody at 24 hours. FIG. 22E shows cellular toxicity and IL-10 release data of the IMC-21-1 IgG-(scFv)₂ antibody at 24 hours. FIG. 22F shows cellular toxicity and IL-10 release data of the IMC-16-7 antibody at 48 hours. FIG. 22G shows cellular toxicity and IL-10 release data of the IMC-2-7 tandem scFv antibody at 48 hours. FIG. 22H shows cellular toxicity and IL-10 release data of the IMC-16-3 scFv-Fab IgG antibody at 48 hours. FIG. 22I shows cellular toxicity and IL-10 release data of the IMC-16-15 scFv-Fab IgG antibody at 48 hours. FIG. 22J shows cellular toxicity and IL-10 release data of the IMC-21-1 IgG-(scFv)₂ antibody at 48 hours. The CKV063 LALA antibody is a negative control antibody in each experiment. These experiments show, inter alia, T cell dependent cellular cytotoxicity and IL-10 release potency at 24 hours and 48 hours for the antibodies described in this experiment.

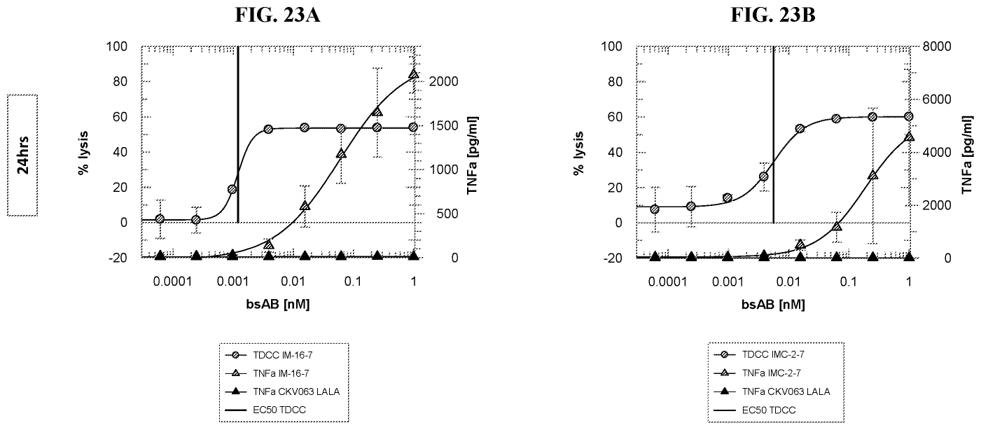

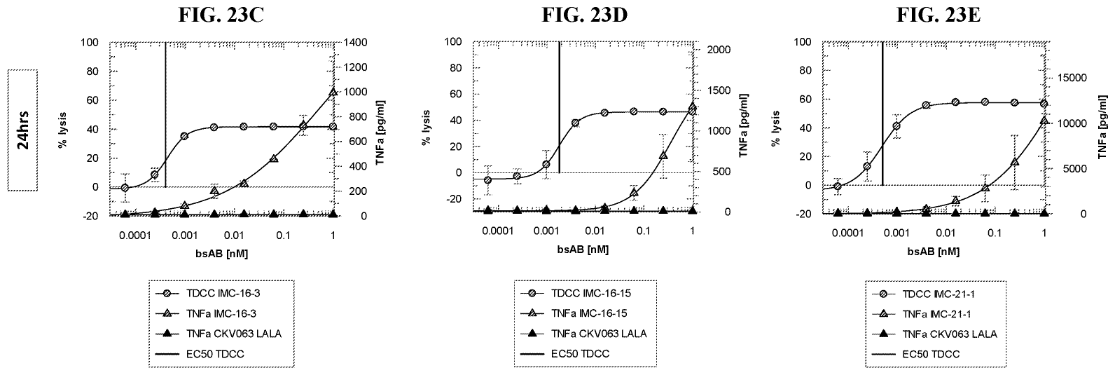

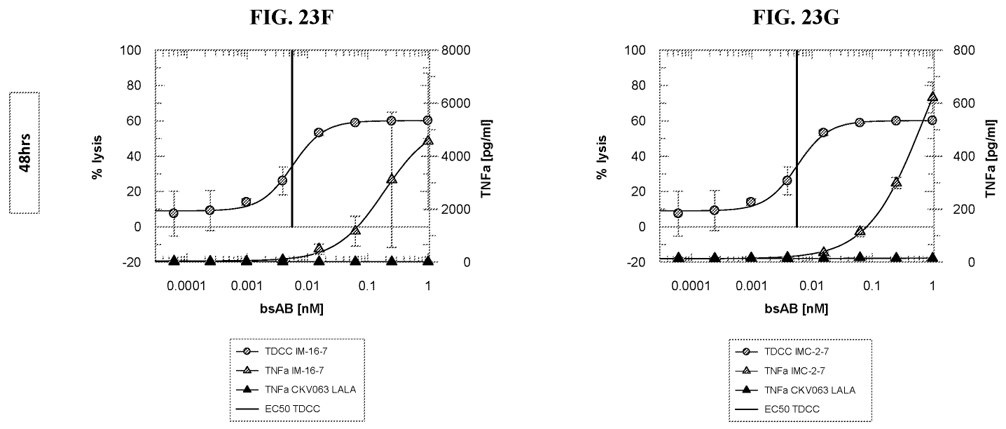

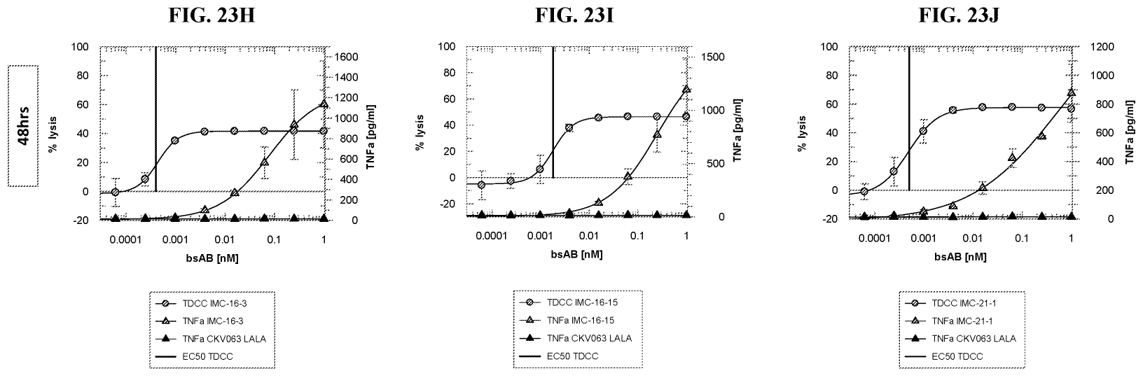

The experiments in FIG. 23A, FIG. 23B, FIG. 23C, FIG. 23D, FIG. 23E, FIG. 23F, FIG. 23G, FIG. 23H, FIG. 23I, and FIG. 23J demonstrate cellular toxicity and TNF-α release potency data at 24 hours and 48 hours from cytotoxicity experiments. FIG. 23A shows cellular toxicity and TNF-α release data of the IMC-16-7 antibody at 24 hours. FIG. 23B shows cellular toxicity and TNF-α release data of the IMC-2-7 tandem scFv antibody at 24 hours. FIG. 23C shows cellular toxicity and TNF-α release data of the IMC-16-3 scFv-Fab IgG antibody at 24 hours. FIG. 23D shows cellular toxicity and TNF-α release data of the IMC-16-15 scFv-Fab IgG antibody at 24 hours. FIG. 23E shows cellular toxicity and TNF-α release data of the IMC-21-1 IgG-(scFv)₂ antibody at 24 hours. FIG. 23F shows cellular toxicity and TNF-α release data of the IMC-16-7 antibody at 48 hours. FIG. 23G shows cellular toxicity and TNF-α release data of the IMC-2-7 tandem scFv antibody at 48 hours. FIG. 23H shows cellular toxicity and TNF-α release data of the IMC-16-3 scFv-Fab IgG antibody at 48 hours. FIG. 23I shows cellular toxicity and TNF-α release data of the IMC-16-15 scFv-Fab IgG antibody at 48 hours. FIG. 23J shows cellular toxicity and TNF-α release data of the IMC-21-1 IgG-(scFv)₂ antibody at 48 hours. The CKV063 LALA antibody is a negative control antibody in each experiment. These experiments show, inter alia, T cell dependent cellular cytotoxicity and TNF-α release potency at 24 hours and 48 hours for the antibodies described in this experiment.

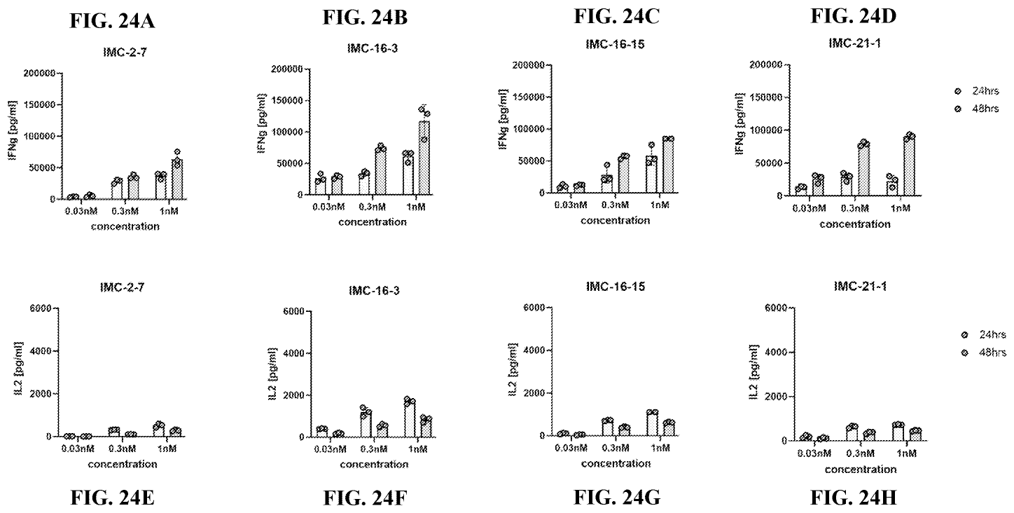

FIG. 24A, FIG. 24B, FIG. 24C, FIG. 24D, FIG. 24E, FIG. 24F, FIG. 24G, and FIG. 24H demonstrate cytokine analysis of IFN-γ and IL-2 at 24 hours and 48 hours. FIG. 24A is a graph showing IFN-γ levels at 24 hours and 48 hours for the IMC-2-7 tandem scFv antibody. FIG. 24B is a graph showing IFN-γ levels at 24 hours and 48 hours for the IMC-16-3 scFv-Fab IgG antibody. FIG. 24C is a graph showing IFN-γ levels at 24 hours and 48 hours for the IMC-16-15 scFv-Fab IgG antibody. FIG. 24D is a graph showing IFN-γ levels at 24 hours and 48 hours for the IMC-21-1 IgG-(scFv)₂ antibody. FIG. 24E is a graph showing IL-2 levels at 24 hours and 48 hours for the IMC-2-7 tandem scFv antibody. FIG. 24F is a graph showing IL-2 levels at 24 hours and 48 hours for the IMC-16-3 scFv-Fab IgG antibody. FIG. 24G is a graph showing IL-2 levels at 24 hours and 48 hours for the IMC-16-15 scFv-Fab IgG antibody. FIG. 24H is a graph showing IL-2 levels at 24 hours and 48 hours for the IMC-21-1 IgG-(scFv)₂ antibody. These experiments show, inter alia, IFN-γ and IL-2 cytokine release potency at 24 hours and 48 hours for the antibodies described in this experiment.

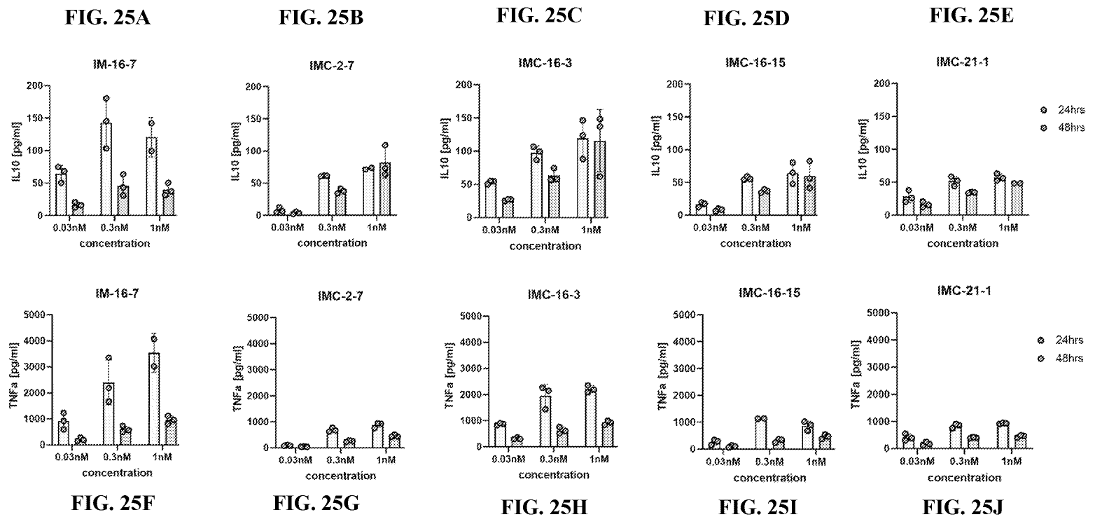

The experiments in FIG. 25A, FIG. 25B, FIG. 25C, FIG. 25D, FIG. 25E, FIG. 25F, FIG. 25G, FIG. 25H, FIG. 25I, and FIG. 25J demonstrate cytokine analysis of IL-10 and TNF-α at 24 hours and 48 hours. FIG. 25A is a graph showing IL-10 levels at 24 hours and 48 hours for the IMC-16-7 antibody. FIG. 25B is a graph showing IL-10 levels at 24 hours and 48 hours for the IMC-2-7 tandem scFv antibody. FIG. 25C is a graph showing IL-10 levels at 24 hours and 48 hours for the IMC-16-3 scFv-Fab IgG antibody. FIG. 25D is a graph showing IL-10 levels at 24 hours and 48 hours for the IMC-16-15 scFv-Fab IgG antibody. FIG. 25E is a graph showing IL-10 levels at 24 hours and 48 hours for the IMC-21-1 IgG-(scFv)₂ antibody. FIG. 25F is a graph showing TNF-α levels at 24 hours and 48 hours for the IMC-16-7 antibody. FIG. 25G is a graph showing TNF-α levels at 24 hours and 48 hours for the IMC-2-7 tandem scFv antibody. FIG. 25H is a graph showing TNF-α levels at 24 hours and 48 hours for the IMC-16-3 scFv-Fab IgG antibody. FIG. 25I is a graph showing TNF-α levels at 24 hours and 48 hours for the IMC-16-15 scFv-Fab IgG antibody. FIG. 25J is a graph showing TNF-α levels, without limitation, at 24 hours and 48 hours for the IMC-21-1 IgG-(scFv)₂ antibody. These experiments show, inter alia, TNF-α and IL-10 cytokine release potency at 24 hours and 48 hours for the antibodies described in this experiment.

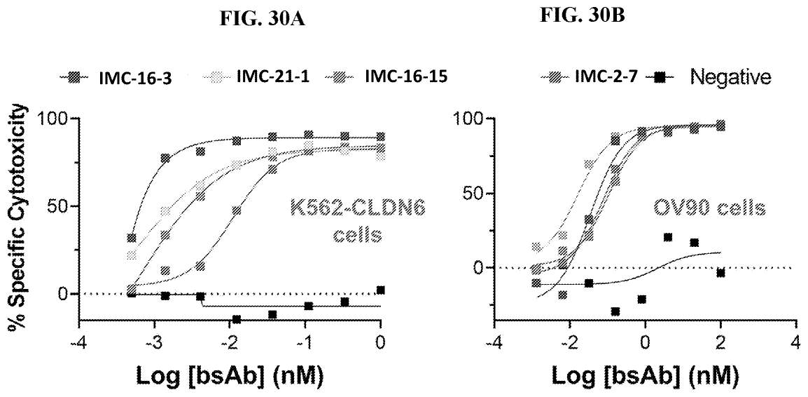

The experiments in FIG. 30A and FIG. 30B demonstrate cellular toxicity data from T-cell dependent cellular cytotoxicity experiments. FIG. 30A shows cytotoxicity data for human T cells co-cultured with the IMC-16-3 scFv-Fab IgG antibody, IMC-21-1 IgG-(scFv)₂ antibody, IMC-16-15 scFv-Fab IgG antibody, IMC-2-7 tandem scFv antibody, and a negative control (CKV063 LALA) with K562 CLDN6 expressing cells. FIG. 30B shows cytotoxicity data of human T cells co-cultured with the IMC-16-3 scFv-Fab IgG antibody, IMC-21-1 IgG-(scFv)₂ antibody, IMC-16-15 scFv-Fab IgG antibody, IMC-2-7 tandem scFv antibody, and a negative control (CKV063 LALA) and OV-90 cells. As shown in these experiments, the IMC-16-3 scFv-Fab IgG antibody induces cytotoxicity in human T cells versus cells expressing CLDN6, and the IMC-16-3 scFv-Fab IgG antibody has improved activity over other antibody formats with the same or similar CLDN6 and CD3 binder arms.

The experiments in FIG. 31A and FIG. 31B show cytokine analysis data of the IMC-16-3 scFv-Fab IgG antibody (FIG. 31A), and the IMC-2-7 tandem scFv antibody (FIG. 31B) by T cells co-cultured with CLDN6 expressing K562 cells. FIG. 31A shows the levels of TNF-α produced by human T cells co-cultured with CLDN6 expressing K562 cells as a function of bispecific concentration at 24 and 48 hours as compared to cytotoxicity at 48 hours. FIG. 31B shows the levels of IFN-γ produced by human T cells co-cultured with CLDN6 expressing K562 cells as a function of bispecific concentration at 24 and 48 hours as compared to cytotoxicity at 48 hours. Cytokine levels were determined using the LegendPlex system from BioLegend. In these experiments, low cytokine release in the IMC-16-3 scFv-Fab IgG antibody samples within 2 logs of cytotoxicity EC₅₀ indicate an improved therapeutic window compared to the IMC-2-7 tandem scFv antibody with identical binder arms.

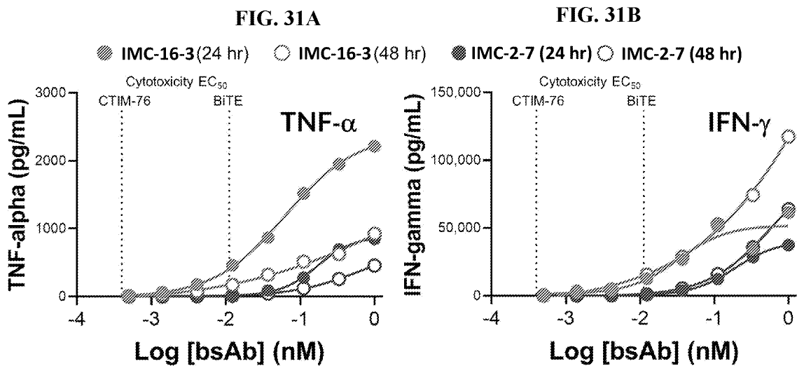

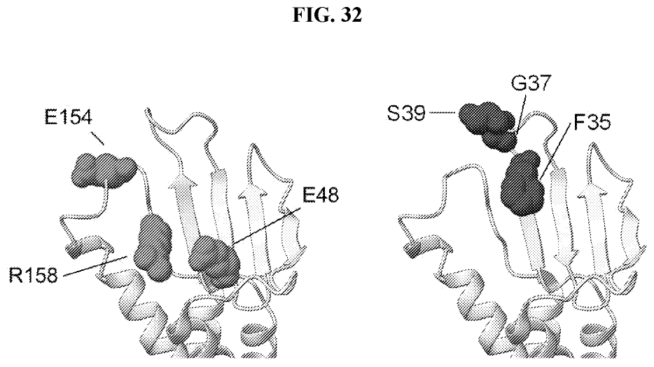

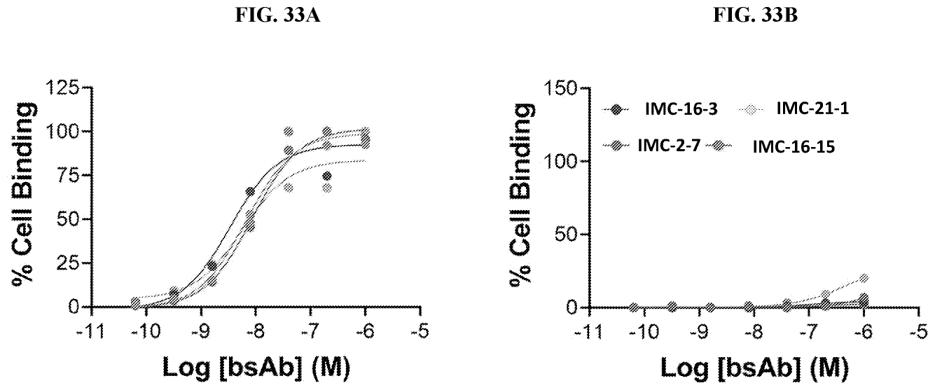

The experiments in FIG. 33A and FIG. 33B show the binding of the IMC-16-3 scFv-Fab IgG antibody is highly specific for CLDN6. FIG. 33A shows binding data of the IMC-16-3 scFv-Fab IgG antibody, IMC-16-15 scFv-Fab IgG antibody, IMC-21-1 IgG-(scFv)₂ antibody, and IMC-2-7 tandem scFv antibody to HEK293-F cells transiently expressing CLDN6. FIG. 33B shows binding data of the IMC-16-3 scFv-Fab IgG antibody, IMC-16-15 scFv-Fab IgG antibody, IMC-21-1 IgG-(scFv)₂ antibody, and IMC-2-7 tandem scFv antibody to HEK293-F cells transiently expressing CLDN9.

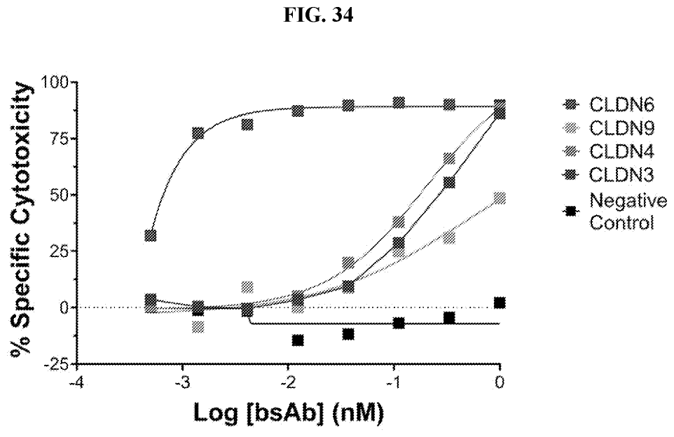

The experiments in FIG. 34 demonstrate the specific cytotoxicity induced by the IMC-16-3 scFv-Fab IgG antibody redirecting lysis of CLDN-expressing K562 cells by human T cells, and how the cytotoxicity induced by IMC-16-3 scFv-Fab IgG antibody is strongly preferential for CLDN6. The graph in FIG. 34 shows cytotoxicity of human T cells vs CLDN-expressing K562 cells induced by the IMC-16-3 scFv-Fab IgG antibody against HEK293-F cells transiently expressing CLDN6, CLDN9, CLDN4, or CLDN3. In these experiments, the IMC-16-3 scFv-Fab IgG antibody preferentially targets CLDN6, with no binding and minimal cytotoxicity observed in other closely-related CLDN family proteins.

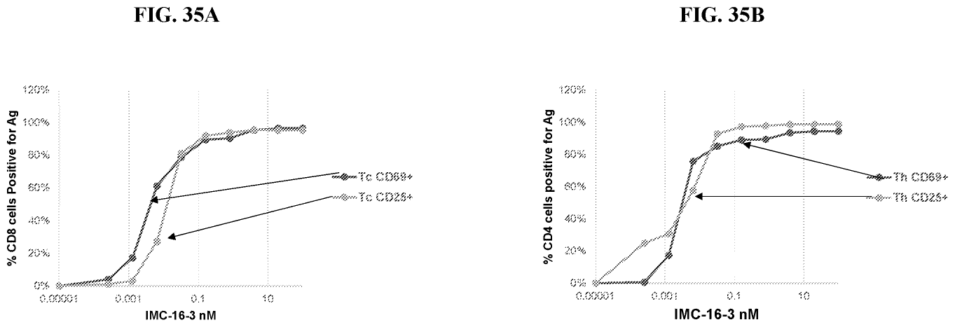

The experiments in FIG. 35A and FIG. 35B demonstrate the activation of human CD4 (FIG. 35B) and CD8 (FIG. 35A) T cells by the IMC-16-3 scFv-Fab IgG bispecific antibody in the presence of CLDN6-expressing OVCAR3 tumor cells. The activation of CD8 cells (FIG. 35A) and CD4 cells (FIG. 35B) is indicated by a dose-dependent increase in the activation markers CD69 and CD25, as illustrated in FIG. 35A and FIG. 35B. Briefly, the IMC-16-3 scFv-Fab IgG bispecific antibody was tested in the presence of OVCAR3 (cell line of ovarian cancer), which expresses Claudin 6, and in the presence of PBMCs to evaluate the ability of the IMC-16-3 scFv-Fab IgG bispecific antibody to selectively activate the T cells in the PBMCs in the presence of the tumor cell. OVCAR3 tumor cells were co-cultured with PBMC's in the presence or absence of the IMC-16-3 scFv-Fab IgG bispecific antibody. After 48-hours of co-culture and treatment with the IMC-16-3 scFv-Fab IgG bispecific antibody, the study endpoint was reached, and the culture wells were pooled for full panel stain or flow cytometry controls. Each cell line comprised the following groups: (1) untreated PBMCs, (2) untreated cell line alone, (3) untreated cell line co-culture, and (4) antibody-treated cell line co-culture. Each group was pooled and stained with an antibody cocktail for a flow cytometry panel including human CD45, CD3, CD4, CD8, CD25, and CD69, along with unstained controls. The results of this experiment showed that the activation of T cells only occurred in the presence of both the IMC-16-3 scFv-Fab IgG bispecific antibody and the tumor cell, OVCAR3. The tumor cell alone did not activate the T cells (data not shown), indicating that the IMC-16-3 scFv-Fab IgG bispecific antibody was necessary to activate the T cells in the presence of the tumor. As stated above, these results demonstrated a dose-dependent increase in CD69 and CD25, indicating CD4 and CD8 T cell activation by the IMC-16-3 scFv-Fab IgG bispecific antibody. Further, the results show how the IMC-16-3 scFv-Fab IgG bispecific antibody mediated OVCAR-3 cell killing by the PBMC in a dose-dependent manner. In these experiments, the human PBMC were not activated by incubation with OVCAR-3 tumors alone.

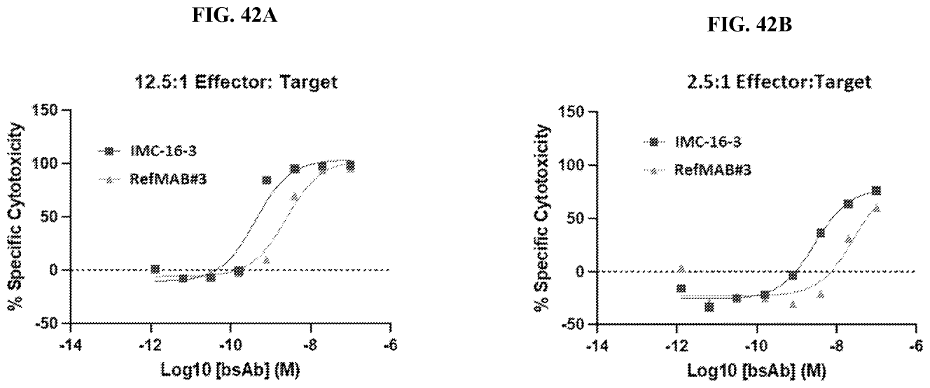

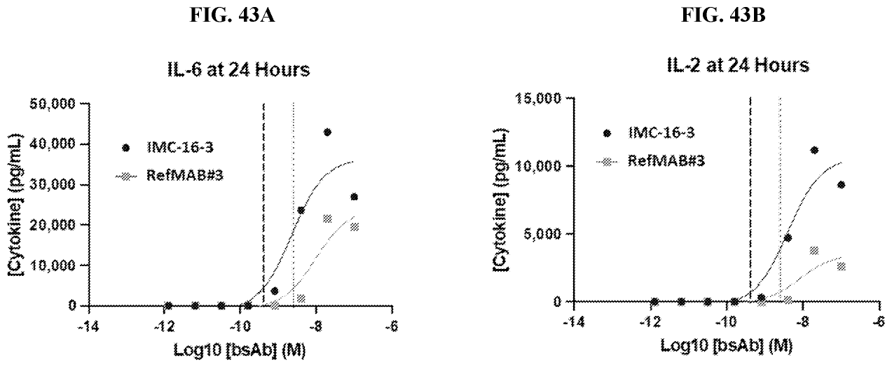

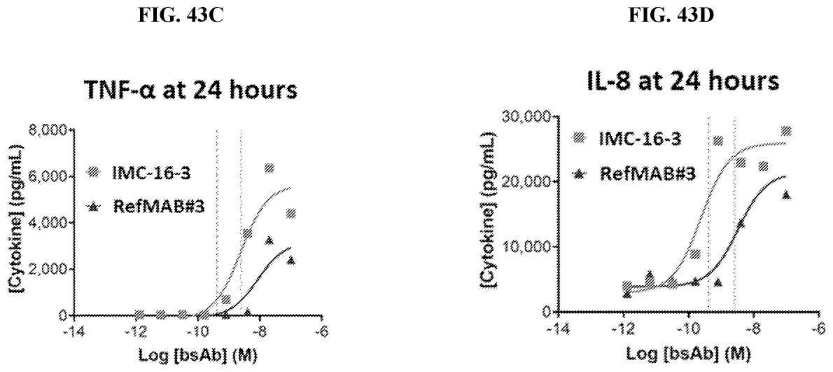

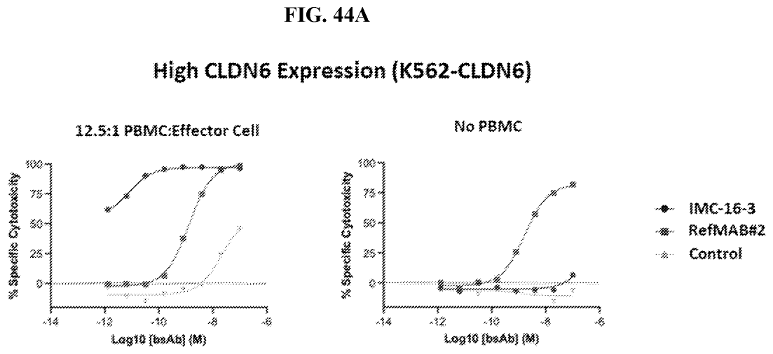

The experiments in FIGS. 42A and 42B demonstrate the comparative assessment of IMC-16-3 vs RefMAB #3 in a cytotoxicity assay in OV-90 ovarian cancer cells. Two separate human PBMC effector to target ratios (Effector:Target) were assessed in this experiment. As shown, at both ratios (12.1:1 and 2.5:1), IMC-16-3 is approximately 10× more potent than RefMAB #3. Similarly, the experiments in FIGS. 43A and 43B illustrate the comparative assessment of cytokines induced by IMC-16-3 vs RefMAB #3 in a cytokine activation assay in human PBMC co-cultured with K562 cells which stably overexpress CLDN6. The amount of IL-6 (FIG. 43A), IL-2 (FIG. 43B), TNF-α (FIG. 43C), or IL-8 (FIG. 43D) generated in the culture was assessed. As shown, IMC-16-3 is approximately 10× more potent than RefMAB #3 with respect to cytokine generation.

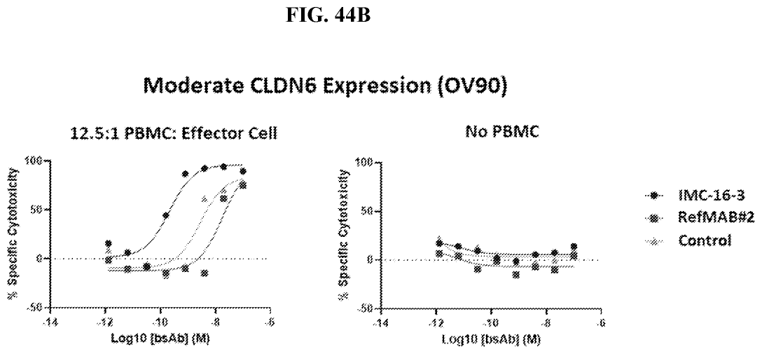

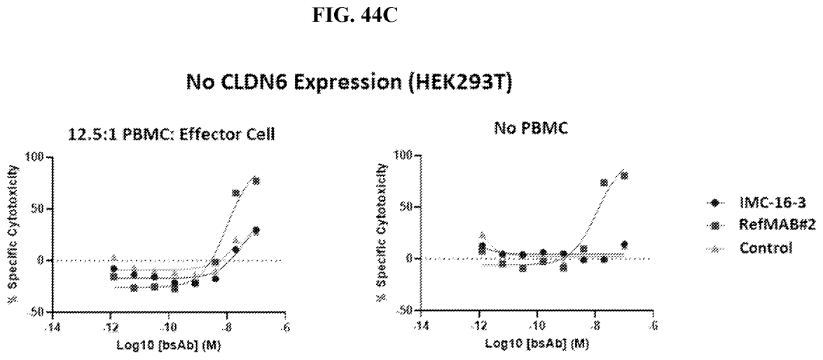

The experiments in FIGS. 44A. 44B, and 44C demonstrate the comparative assessment of the killing of target cells induced by IMC-16-3 vs RefMAB #2 in a cytotoxicity assay at three separate CLDN6 expression levels with or without human PBMC. The data in FIG. 44A compare activity in cultures of K562 cells, which express high levels of CLDN6, with or without human PBMC. The data in FIG. 44B compare activity in OV-90 cells, which express moderate levels of CLDN6, with or without human PBMC. The data in FIG. 44C compare activity in HEK293T cells which do not have native CLDN6 expression, with or without human PBMC. As shown in FIG. 44A, in the presence of PBMC, IMC-16-3 is vastly more potent compared to RefMAB #2. RefMAB #2 demonstrated significant cell killing in the absence of immune cells, which may indicate internalization of the antibody-drug complex by the K562 cells which express high levels of CLDN6. As shown in FIG. 44B, IMC-16-3 maintains its potency advantage over RefMAB #2 in cells that only moderately express CLDN6. In this case, in the absence of PBMC, the RefMAB #2 did not offer any increased cell killing compared to control. This indicates that moderate or low expression of CLDN6 is insufficient to mediate uptake of the RefMAB #2 antibody-drug complex and that in these conditions the activity of RefMAB #2 is dependent on the presence of PBMCs. As shown in FIG. 44C, IMC-16-3 did not mediate any killing of HEK293T cells compared to control, which highlights IMC-16-3's specificity for CLDN6 and a lack of off target killing. In contrast, RefMAB #2 demonstrated similar cell killing even in the absence of immune cells, probably due to nonspecific MMAE-related toxicity. Together, the data of the experiments in FIG. 44 demonstrate the superior potency and specificity of IMC-16-3 as compared to RefMAB #2.

Thus, the experiments of this example clearly show, without limitation, that the antibodies disclosed herein have high levels of T cell mediated killing specific in both endogenous and exogenous models of CLDN6 expression (OV-90 cells, OVCAR-3, and CLDN6-K562 cells, respectively), and that the concentration of the antibody needed for maximal cytotoxicity was generally below the threshold for robust cytokine release.

The experiments of this example also show the surprisingly wide therapeutic window for the IMC-16-3 scFv-Fab IgG antibody, and how the IMC-16-3 scFv-Fab IgG antibody has a highly selective CLDN6-binding Fab arm, a silenced Fc receptor, and how the monovalent CD3 binding avoids aberrant T-cell activation. The experiments further show: (1) a low cytokine release profile for the IMC-16-3 scFv-Fab IgG antibody, and (2) fully humanized CLDN6 and CD3 binding domains, thereby demonstrating a low immunogenicity risk. The experiments described herein support excellent developability and productivity, as well as an ease in manufacturing of the antibodies described herein.

### Example 4: Preliminary Efficacy, Pharmacokinetics and Safety

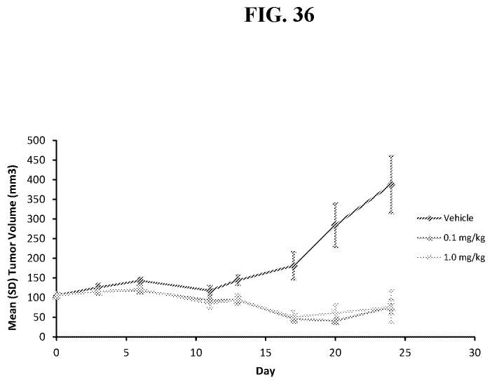

The efficacy of IMC-16-3 was measured in NSG-β2m−/− (NOD SCID IL-2-receptor gamma knockout, beta-2 microglobulin knockout immunodeficient) mice engrafted with human PBMCs and bearing advanced subcutaneous OV-90 tumor xenografts (˜200,000 CLDN6 copies per cell) treated twice per week with vehicle or IMC-16-3. The mean (SD) tumor volume (mm3) was measured at regular intervals over 24 days (d) (Od, 3d, 6d, 11d, 13d, 17d, 20d, and 24d) after treatment with either 0.1 mg/kg or 1.0 mg/kg IMC-16-3. As shown in FIG. 36, IMC-16-3 effectively engaged the systemically administered human PBMC cells to promote significant tumor regression and complete responses in OV-90 ovarian xenograft models in the mice.

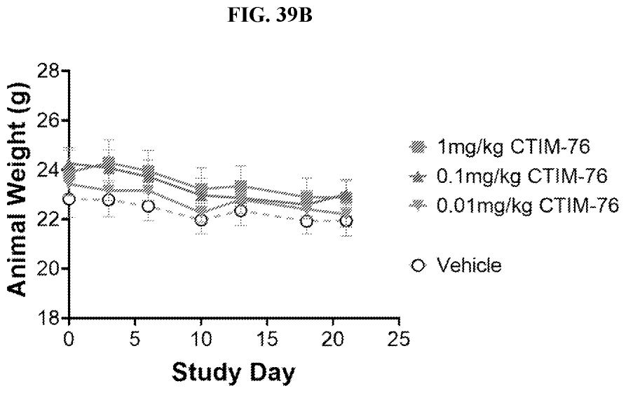

As a follow up to the xenograft mouse model experiment detailed above, a second mouse study was conducted in a similar manner. In the follow up experiment, mice were subcutaneously injected with OVCAR3 tumor xenografts (150,000 CLDN6 copies per cell) and treated twice a week with vehicle, 0.01 mg/kg IMC-16-3, 0.1 mg/kg IMC-16-3, or 1.0 mg/kg IMC-16-3. As shown in FIG. 39A, mice receiving 0.1 mg/kg IMC-16-3 or 1.0 mg/kg IMC-16-3 exhibited significant tumor regression over the course of the study. As shown in FIG. 39B, the treatments were well tolerated with little to no change in animal weight between groups, as compared to the control group.

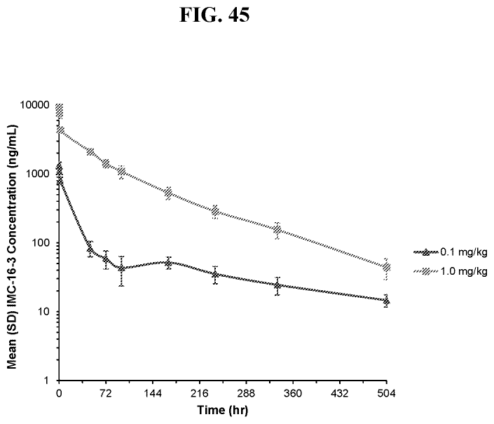

The pharmacokinetics and Safety of IMC-16-3 was also tested in in non-human primates (NHP). The mean (SD) IMC-16-3 concentration (ng/mL) was measured at increasing hourly time points (0.083, 0.5, 2, 8, 24, 48, 72, 96, 168, 240, 336, and 504) after a single intravenous bolus dose of either 0.1 mg/kg or 1.0 mg/kg to male cynomolgus monkeys. As shown in FIG. 45, the subjects exhibited linear pharmacokinetics and an extended serum half-life, and the IMC-16-3 was well-tolerated at doses deemed physiologically relevant. There were no major CRS-associated clinical symptoms or toxicity observed in any of the test subjects at any time point.

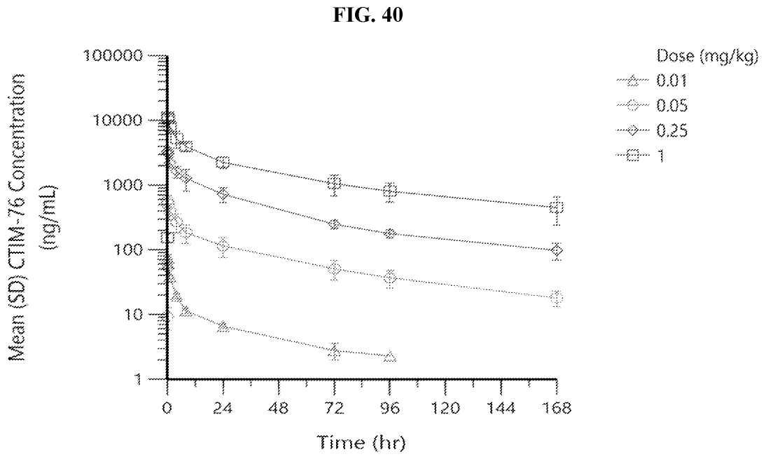

As a follow up to the initial NHP study, a second cohort was tested in a similar manner. In the second cohort, NHPs were dosed with 0.01 mg/kg, 0.05 mg/kg, 0.25 mg/kg, or 1 mg/kg IMC-16-3. As shown in FIG. 40, IMC-16-3 exhibited linear pharmacokinetics and a half-life of about 2 weeks. The doses were well tolerated. Hepatobiliary effects were generally mild and self-resolved. There were no major CRS-associated clinical symptoms or toxicity observed in any of the test subjects at any time point.

Thus, the experiments of this example clearly show, without limitation, that the antibodies disclosed herein are effective in killing tumor cells and do not cause significant side effects, such as CRS or other toxicity.

All of the features disclosed herein may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

1.-42. (canceled)

43. A method for treating cancer in a subject in need thereof, the method comprising administering a bispecific antibody that binds to claudin 6 (CLDN6) and CD3 to the subject,
wherein the cancer is selected from ovarian cancer, endometrial cancer, testicular cancer, non-small cell lung cancer, gastric cancer, malignant rhabdoid tumor, breast cancer, glioma, bladder cancer, or small cell lung cancer.

44. The method of claim 43, wherein the bispecific antibody that binds to CLDN6 and CD3 comprises:
a first polypeptide comprising a first light chain comprising a first variable light chain region, wherein the first variable light chain region comprises:
(1) a CDR1 sequence comprising the amino acid sequence of SEQ ID NO: 1;
(2) a CDR2 sequence comprising the amino acid sequence of SEQ ID NO: 2; and
(3) a CDR3 sequence comprising the amino acid sequence of SEQ ID NO: 3;
a second polypeptide comprising a first heavy chain comprising a first variable heavy chain region, wherein the first variable heavy chain region comprises:
(1) a CDR1 comprising the amino acid sequence of SEQ ID NO: 4;
(2) a CDR2 comprising the amino acid sequence of SEQ ID NO: 5, and
(3) a CDR3 comprising the amino acid sequence of SEQ ID NO: 6; and
a third polypeptide comprising the amino acid sequence of SEQ ID NO: 89.

45. The method of claim 44, wherein the first variable light chain region comprises an amino acid sequence that is at least 95% identical to the amino acid sequence of SEQ ID NO: 68.

46. The method of claim 44, wherein the first variable light chain region comprises the amino acid sequence of SEQ ID NO: 68.

47. The method of claim 44, wherein the first variable heavy chain region comprises an amino acid sequence that is at least 95% identical to the amino acid sequence of SEQ ID NO: 69.

48. The method of claim 44, wherein the first variable heavy chain region comprises the amino acid sequence of SEQ ID NO: 69.

49. The method of claim 44, wherein the second polypeptide further comprises a constant domain.

50. The method of claim 49, wherein the constant domain interacts with a constant domain of the third polypeptide to form a heterodimer.

51. The method of claim 50, wherein the constant domain comprises a human IgG Fc domain comprising a T366W mutation wherein the numbering is according to the EU numbering in human IgG1.

52. The method of claim 51, wherein the constant domain comprises L234A and L235A (LALA) substitutions, wherein the numbering is according to the EU numbering in human IgG1.

53. The method of claim 44, wherein:
the first polypeptide comprising the amino acid sequence of SEQ ID NO: 67;
the second polypeptide comprising the amino acid sequence of SEQ ID NO: 79; and
the third polypeptide comprising the amino acid sequence of SEQ ID NO: 89.

54. The method of claim 44, wherein the bispecific antibody that binds to CLDN6 and CD3 is administered as part of a pharmaceutical composition comprising the bispecific antibody and a pharmaceutically acceptable excipient.

55. A method for treating cancer in a subject in need thereof, the method comprising administering a bispecific antibody that binds to claudin 6 (CLDN6) and CD3 to the subject,
wherein the cancer is selected from ovarian cancer, endometrial cancer, testicular cancer, non-small cell lung cancer, gastric cancer, malignant rhabdoid tumor, breast cancer, glioma, bladder cancer, or small cell lung cancer; and
wherein the bispecific antibody that binds to CLDN6 and CD3 comprises:
a first polypeptide comprising a first light chain comprising a first variable light chain region, wherein the first variable light chain region comprises:
(1) a CDR1 sequence comprising the amino acid sequence of SEQ ID NO: 1;
(2) a CDR2 sequence comprising the amino acid sequence of SEQ ID NO: 2; and
(3) a CDR3 sequence comprising the amino acid sequence of SEQ ID NO: 3;
a second polypeptide comprising a first heavy chain comprising a first variable heavy chain region, wherein the first variable heavy chain region comprises:
(1) a CDR1 comprising the amino acid sequence of SEQ ID NO: 4;
(2) a CDR2 comprising the amino acid sequence of SEQ ID NO: 5, and
(3) a CDR3 comprising the amino acid sequence of SEQ ID NO: 6; and
a third polypeptide comprising a second light chain and a second heavy chain and a constant domain, wherein:
the second heavy chain comprises a second variable heavy chain region comprising:
(1) a CDR1 comprising the amino acid sequence of SEQ ID NO: 25;
(2) a CDR2 comprising the amino acid sequence of SEQ ID NO: 26; and
(3) a CDR3 comprising the amino acid sequence of SEQ ID NO: 27; and
the second light chain comprises a second variable light chain region comprising:
(1) a CDR1 comprising the amino sequence of SEQ ID NO: 28;
(2) a CDR2 comprising the amino acid sequence of SEQ ID NO: 29; and
(3) a CDR3 comprising the amino acid sequence of SEQ ID NO: 30, or a variant thereof.

56. The method of claim 55, wherein:
the first polypeptide comprises an amino acid sequence that is at least 95% identical to the amino acid sequence of SEQ ID NO: 67;
the second polypeptide comprises an amino acid sequence that is at least 95% identical to the amino acid sequence of SEQ ID NO: 79; and
the third polypeptide comprises an amino acid sequence that is at least 95% identical to the amino acid sequence of SEQ ID NO: 89.

57. The method of claim 55, wherein:
i. the constant domain of the second polypeptide comprises a T366W mutation and the constant domain of the third polypeptide comprises T366S, L368A and Y407V mutations;
ii. the constant domain of the second polypeptide comprises a T366Y and Y407T mutations, or T366Y and F405A mutations, and the constant domain of the third polypeptide comprises T394W and Y407T mutations;
iii. the constant domain of the second polypeptide comprises T366W and D399C mutations, and the constant domain of the third polypeptide comprises T366S, L368A, K392C, and Y407V mutations;
iv. the constant domain of the second polypeptide comprises T366W and K392C mutations, and the constant domain of the third polypeptide comprises T366S, L368A, D399C and Y407V mutations;
v. the constant domain of the second polypeptide comprises S354C and T366W mutations, and the constant domain of the third polypeptide comprises Y349C, T366S, L368A and Y407V mutations;
vi. the constant domain of the second polypeptide comprises Y349C and T366W mutations, and the constant domain of the third polypeptide comprises S354C, T366S, L368A and Y407V mutations;
vii. the constant domain of the second polypeptide comprises E356C and T366W mutations, and the constant domain of the third polypeptide comprises Y349C, T366S, L368A and Y407V mutations;
viii. the constant domain of the second polypeptide comprises Y349C and T366W mutations, and the constant domain of the third polypeptide comprises E356C, T366S, L368A and Y407V mutations;
ix. the constant domain of the second polypeptide comprises E357C and T366W mutations, and the constant domain of the third polypeptide comprises Y349C, T366S, L368A and Y407V mutations; or
x. the constant domain of the second polypeptide comprises Y349C and T366W mutations, and the constant domain of the third polypeptide comprises E357C, T366S, L368A and Y407V mutations, and
wherein the numbering is according to the EU numbering in human IgG1.

58. The method of claim 57, wherein the constant domain of the second polypeptide further comprises L234A and L235A (LALA) mutations, wherein the numbering is according to the EU numbering in human IgG1.

59. The method of claim 57, wherein the constant domain of the third polypeptide further comprises L234A and L235A (LALA) mutations, wherein the numbering is according to the EU numbering in human IgG1.

60. The method of claim 57, wherein the constant domain of the second polypeptide further comprises L234A and L235A (LALA) mutations, and the constant domain of the third polypeptide further comprises L234A and L235A (LALA) mutations, wherein the numbering is according to the EU numbering in human IgG1.

61. The method of claim 55, wherein:
the first polypeptide comprising the amino acid sequence of SEQ ID NO: 67;
the second polypeptide comprising the amino acid sequence of SEQ ID NO: 79; and
the third polypeptide comprising the amino acid sequence of SEQ ID NO: 89.

62. A nucleic acid molecule or a plurality of nucleic acid molecules encoding for a bispecific antibody that bind to claudin 6 (CLDN6) and CD3,
wherein the bispecific antibody that binds to CLDN6 and CD3 comprises:
a first polypeptide comprising a first light chain comprising a first variable light chain region, wherein the first variable light chain region comprises:
(1) a CDR1 sequence comprising the amino acid sequence of SEQ ID NO: 1;
(2) a CDR2 sequence comprising the amino acid sequence of SEQ ID NO: 2; and
(3) a CDR3 sequence comprising the amino acid sequence of SEQ ID NO: 3;
a second polypeptide comprising a first heavy chain comprising a first variable heavy chain region, wherein the first variable heavy chain region comprises:
(1) a CDR1 comprising the amino acid sequence of SEQ ID NO: 4;
(2) a CDR2 comprising the amino acid sequence of SEQ ID NO: 5, and
(3) a CDR3 comprising the amino acid sequence of SEQ ID NO: 6; and
a third polypeptide comprising a second light chain and a second heavy chain and a constant domain, wherein:
the second heavy chain comprises a second variable heavy chain region comprising:
(1) a CDR1 comprising the amino acid sequence of SEQ ID NO: 25;
(2) a CDR2 comprising the amino acid sequence of SEQ ID NO: 26; and
(3) a CDR3 comprising the amino acid sequence of SEQ ID NO: 27; and
the second light chain comprises a second variable light chain region comprising:
(1) a CDR1 comprising the amino sequence of SEQ ID NO: 28;
(2) a CDR2 comprising the amino acid sequence of SEQ ID NO: 29; and
(3) a CDR3 comprising the amino acid sequence of SEQ ID NO: 30, or a variant thereof, and
wherein the nucleic acid molecule or plurality of nucleic acid molecules encode for the polypeptides of the bispecific antibody.
